SEQUENCE LISTING (8 sequences)

SEQ ID NO: 1 - DNA, 20 bp, Artificial Sequence
  SYNTHETIC PRIMER
ggggccagcgtttctttttc

SEQ ID NO: 2 - DNA, 20 bp, Artificial Sequence
  SYNTHETIC PRIMER
aaacatggagacggtgctgg

SEQ ID NO: 3 - DNA, 20 bp, Artificial Sequence
  SYNTHETIC PRIMER
atagctactcacagccaggt

SEQ ID NO: 4 - DNA, 20 bp, Artificial Sequence
  SYNTHETIC PRIMER
gaaggggagaaagacggatc

SEQ ID NO: 5 - DNA, 26 bp, Artificial Sequence
  SYNTHETIC PRIMER
ttcaaaattgtccacgacatcagctc

SEQ ID NO: 6 - DNA, 18 bp, Artificial Sequence
  SYNTHETIC PRIMER
gccttcacgggtaacggc

SEQ ID NO: 7 - DNA, 21 bp, Artificial Sequence
  SYNTHETIC PRIMER
ttcctaccttagcgctgagcg

SEQ ID NO: 8 - DNA, 18 bp, Artificial Sequence
  SYNTHETIC PRIMER
agggtcagcagggcagaa

(19) United States
(12) Patent Application Publication
(10) Pub. No.: US 2024/0190872 A1
(43) Pub. Date: Jun. 13, 2024

(54) FABI INHIBITORS FOR GRAM-NEGATIVE PATHOGENS

(71) Applicants: THE BOARD OF TRUSTEES OF THE UNIVERSITY OF ILLINOIS, Urbana, IL (US); THE GENERAL HOSPITAL CORPORATION, Boston, MA (US); THE BROAD INSTITUTE, INC., Cambridge, MA (US)

(72) Inventors: Paul J. HERGENROTHER, Champaign, IL (US); Erica Nicole PARKER, Urbana, IL (US); Deborah HUNG, Cambridge, MA (US); Michael SERRANO-WU, Belmont, MA (US); Katie Kyungae LEE, Boston, MA (US)

(73) Assignees: THE BOARD OF TRUSTEES OF THE UNIVERSITY OF ILLINOIS, Urbana, IL (US); THE GENERAL HOSPITAL CORPORATION, Boston, MA (US); THE BROAD INSTITUTE, INC., Cambridge, MA (US)

(21) Appl. No.: 18/548,580

(22) PCT Filed: Mar. 2, 2022
(86) PCT No.: PCT/US22/18472

§ 371 Date: Sep. 1, 2023

Related U.S. Application Data
(60) Provisional application No. 63/156,145, filed on Mar. 3, 2021.

(51) Int. Cl. C07D 471/04; A61K 9/00; A61K 9/20; A61K 9/48; A61K 47/02; A61K 47/06; A61K 47/10; A61K 47/12; A61K 47/14; A61K 47/18; A61K 47/26; A61K 47/32; A61K 47/38; A61K 47/44; A61P 31/04; C07D 471/20

(52) U.S. Cl. CPC C07D 471/04; A61K 9/0019; A61K 9/0073; A61K 9/2013; A61K 9/2018; A61K 9/2027; A61K 9/2054; A61K 9/2059; A61K 9/4858; A61K 9/4866; A61K 47/02; A61K 47/06; A61K 47/10; A61K 47/12; A61K 47/14; A61K 47/18; A61K 47/26; A61K 47/32; A61K 47/38; A61K 47/44; A61P 31/04; C07D 471/20

(57) ABSTRACT

A FabI inhibitor called fabimycin that has impressive activity against >200 clinical isolates of E. coli, K. pneumoniae, and A. baumannii. Fabimycin has activity in multiple mouse models of infection caused by Gram-negative bacteria, including a model of urinary tract infection. Fabimycin has translational promise, and its discovery provides data indicating that antibiotics whose spectrum of activity is restricted to Gram-positive bacteria can be systematically modified to accumulate in Gram-negative bacteria and be effective against these problematic pathogens.

### RELATED APPLICATIONS

This application claims priority under 35 U.S.C. § 119(e) to U.S. Provisional Patent Application No. 63/156,145, filed March 3, 2021, which is incorporated herein by reference.

### GOVERNMENT SUPPORT

This invention was made with government support under Grant No. AI136773 awarded by the National Institutes of Health. The government has certain rights in the invention.

### BACKGROUND OF THE INVENTION

The discovery and development of novel antibiotic classes for infections caused by Gram-positive pathogens has been a medicinal chemistry success story over the last 20 years, with drugs in the oxazolidinone, mutilin, and lipopeptide classes all having notable clinical impact. Further, there are additional antibiotics moving through clinical trials for Gram-positive infections, including new compound classes and antibiotics that engage unexploited biological targets. In contrast, there has not been a novel class of antibiotics FDA approved for treatment of Gram-negative ESKAPE pathogens in over 50 years; this situation has led to increased mortality, with some studies showing that 75% of deaths from drug-resistant pathogens are now caused by Gram-negative infections. This discovery void is largely due to the low likelihood that a given compound will accumulate inside Gram-negative bacteria, as their dense lipopolysaccharide outer membrane and promiscuous efflux pumps work in concert to prevent candidate antibiotics from reaching their target. Recent studies reveal that accumulating compounds often possess a specific combination of physicochemical properties, explaining why high-throughput screens of millions of compounds have failed to identify Gram-negative active antibiotics.

Encouragingly, the same biological processes that make Gram-positive bacteria susceptible to antibiotics can typically be exploited to kill Gram-negative bacteria; as such, inhibitors of protein translation, DNA gyrase, and cell wall biosynthesis have broad-spectrum (Gram-positive and Gram-negative) activity provided they can enter the cell and reach their target. However, many other promising biological targets have not yet been exploited to kill Gram-negative organisms, as the antibiotics that engage these targets do not accumulate in Gram-negative bacteria. One such outstanding target is the enoyl-acyl carrier reductase enzyme FabI, which catalyzes the key rate-determining step in bacterial fatty acid biosynthesis. A lead compound identified in a biochemical screen for FabI inhibition was optimized into Debio-1452, the phosphate prodrug version of which (afabicin) is in Phase 2 clinical trials for infections caused by S. aureus. Even though FabI is a promising exploitable target for problematic Gram-negative ESKAPE pathogens, including E. coli, K. pneumonia, and A. baumannii, Debio-1452 does not accumulate inside these cells and is therefore inactive against these bacteria.

Therefore, there exists a need for novel antibiotics that can accumulate inside Gram-negative pathogens for effective treatment of bacterial infections.

### SUMMARY

We recently utilized an emerging understanding of the connection between physicochemical traits and accumulation in Gram-negative bacteria to design a version of Debio-1452 that is effective against these pathogens. This compound, Debio-1452-NH3, has antibacterial activity against Gram-negative clinical isolates and efficacy in mouse infection models, and as such is the first member of this class to have notable Gram-negative antibacterial activity (Nat Microbiol 2020, 5 (1), 67). Towards the development of a FabI inhibitor that is a true clinical candidate for Gram-negative infections, we now report the use of iterative compound synthesis, clinical isolate testing, and x-ray crystallography to identify fabimycin, a FabI inhibitor with enhanced antibacterial potency, decreased in vivo toxicity, and efficacy in infection models including a challenging mouse model of urinary tract infection (UTI) and several neutropenic murine models of infections with Gram-negative bacteria.

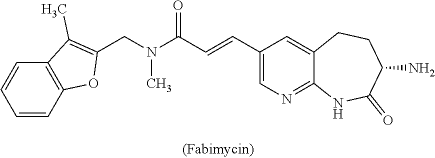 [SMILES: Cc1c(CN(C)C(=O)/C=C/c2cnc3c(c2)CC[C@H](N)C(=O)N3)oc2ccccc12]

Provided herein are novel compounds of any one of Formulas I, IA, IB, IC, ID, II, III, IIIB, IV, or a pharmaceutically acceptable salt thereof. For example, this disclosure provides a compound of Formula I or II:

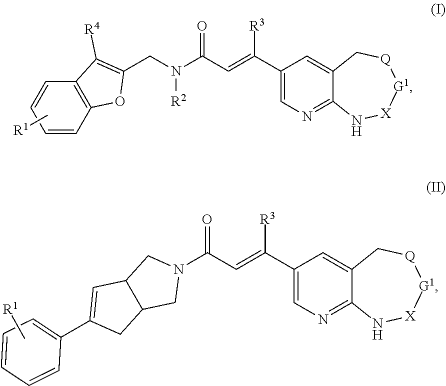

or a salt thereof; wherein

G¹ is —CH(C₀-C₆)alkyl-NH₂, CH₂, —CH(C₃-C₆)cycloalkyl-NH₂, —CHN(H)(C₂-C₆)alkyl-NH₂, or G²;

G² is

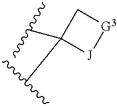

wherein G³ is NH, —CH(C₀-C₆)alkyl-NH₂, or —CH₂CH(C₀-C₆)alkyl-NH₂;
J is —(CH₂)ₘ—; and m is 1-3;

Q is CHR^{a}, NH, —NHCH₂—, —CH₂CH₂—, —C(═O)N—, or absent, wherein R^{a} is H or halo;

X is C═O or CH₂;

R¹ is H or halo;

R² is —(C₁-C₆)alkyl, H, or —(C₁-C₆)alkyl-NH₂;

R³ is H or —(C₁-C₆)alkyl-NH₂; and

R⁴ is methyl, ethyl, propyl, butyl, pentyl, hexyl, or —(C₃-C₆)cycloalkyl;

wherein for Formula I, R² and R⁴ taken together optionally form a heterocycle;
wherein at least one of G¹, R², or R³ comprises an amine moiety and each alkyl moiety is optionally substituted.

In another aspect, provided herein is a method of antimicrobial treatment, comprising, administering to a subject in need thereof a therapeutically effective amount of a compound of any one of Formulas I, IA, IB, IC, ID, II, III, IIIB, IV, or a pharmaceutically acceptable salt thereof, thereby killing or inhibiting the growth of at least a portion of a plurality of microorganisms in the subject, for example, when the subject has a urinary tract infection (UTI).

In another aspect, provided herein is a method of antimicrobial treatment, comprising providing a sample comprising a plurality of microorganisms; and contacting the sample with a compound disclosed herein; thereby killing or inhibiting the growth of at least a portion of the plurality of microorganisms in the sample.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Antimicrobial activity of fabimycin against clinical isolates. A) The susceptibility of clinical isolates (of Gram-negative species and S. aureus) to fabimycin, Debio-1452-NH3, and Debio-1452. B) Further exploration of the breadth of antibacterial activity of fabimycin against diverse clinical isolate panels of K. pneumoniae and A. baumannii, as compared to levofloxacin.

FIG. 2. Fabimycin mode of action studies. A) In vitro inhibition of enzymatic activity of FabI (from A. baumannii and E. coli) by fabimycin ((S)-11) and (R)-11. B) Spontaneous resistance frequency of E. coli MG1655 to fabimycin, and the resulting mutation in FabI in the resistant strains. The mutation prevention concentration (MPC) for fabimycin is 64 μg/mL.

FIG. 3. In-vivo efficacy of fabimycin compared to Debio-1452-NH3. A) Assessment of fabimycin stability in mammalian plasma. B) Neutropenic mouse thigh infection initiated in CD-1 mice with A. baumannii AR-299 (1.22×10⁶ per mouse intramuscular in thigh) were treated with vehicle (8 mice) or FabI inhibitor (8 mice per group) 2, 6, and 11 h post-infection (50 mg/kg intramuscular), and the bacterial burden was evaluated 26 h post-infection. C) Acute pneumonia infections initiated in CD-1 mice with A. baumannii AR-299 (1.6×10⁸ per mouse intranasally) were treated with vehicle (8 mice) or FabI inhibitor (8 mice per group) QD at 50 mg/kg intramuscular injection and the bacterial burden evaluated at 48 h post-infection.

FIG. 4. In-vivo efficacy of fabimycin in a murine UTI. After inducing diuresis, infection initiated in C3H/HeJ mice (8 per arm, 1.38×10⁹ CFU/mouse transurethral) with E. coli AR-55 and treated with fabimycin at varying concentrations three times daily with bacterial enumeration at 168 h post-infection. Percentage in parenthesis indicates the percentage of animals with bacterial counts below the limit of detection (LOD).

FIG. 5. Accumulation of (R)-11 and (S)-11 (fabimycin) in E. coli sp.

FIG. 6. Pharmacokinetic analysis of fabimycin.

FIG. 7. In-vivo efficacy of fabimycin. A) Neutropenic mouse thigh infection model initiated in female BALB/c mice with A. baumannii AR-88 (1.36×10⁵ CFU per mouse intramuscular, left thigh) were treated with vehicle (5 mice) or fabimycin (10 mice) four times a day (75 mg/kg intravenously) and bacterial burden evaluated at 26 h post-infection. B) Neutropenic mouse thigh infection initiated in CD-1 mice with S. aureus USA300 LAC (2.3×10⁶ CFU per mouse intramuscular in thigh) were treated with vehicle (8 mice) or FabI inhibitor (8 mice per group) 2 and 7 h post-infection (5 mg/kg retro-orbital IV), and the bacterial burden was evaluated 24 h post-infection.

FIG. 8. Fabimycin dose fractionation in UTI evaluated in mouse tissue. Impact of various dosing regimens with enpamcyin at 168 h post-infection with E. coli AR-55. A) Bladder; B) Spleen; C) Liver; D) Kidneys.

FIG. 9. Fabimycin dose fractionation in UTI evaluated in mouse urine.

### DETAILED DESCRIPTION

Genomic studies and experiments with permeability deficient strains have revealed a wide variety of biological targets that can be engaged to kill Gram-negative bacteria. However, the formidable outer membrane and promiscuous efflux pumps of these pathogens act to keep many candidate antibiotics from reaching these targets. One such promising target is the enzyme FabI, which catalyzes a key committed step in bacterial fatty acid biosynthesis; FabI inhibitors have advanced to clinical trials for Gram-positive infections (specifically S. aureus), but not for infections caused by Gram-negative bacteria. Here we synthesize a suite of putative FabI inhibitors whose structures fit permeation rules for Gram-negative bacteria, and use activity against a challenging panel of Gram-negative clinical isolates as a key filter for advancement. The compound to emerge from this process, called fabimycin, has impressive activity against >200 clinical isolates of E. coli, K. pneumoniae, and A. baumannii. X-ray crystal data in complex with the E. coli and A. baumannii versions of FabI suggests how fabimycin is able to inhibit FabI more potently than its opposite enantiomer. Importantly, fabimycin has activity in multiple mouse models of infection caused by Gram-negative bacteria, including a model of urinary tract infection. Fabimycin has translational promise, and its discovery provides another piece of data suggesting that antibiotics whose spectrum of activity is restricted to Gram-positive bacteria can be systematically modified to accumulate in Gram-negative bacteria and be effective against these problematic pathogens.

Additional information and data supporting the invention can be found in the following publications by the inventors: WO2019177975 and WO2017156519, which publications are incorporated herein by reference in their entirety.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art, especially in light of the detailed disclosure provided herein. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, the terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods provided herein, the mammal is a human

As used herein, the terms “providing”, “administering,” “introducing,” are used interchangeably herein and refer to the placement of the compositions of the disclosure into a subject by a method or route which results in at least partial localization of the composition to a desired site. The compositions can be administered by any appropriate route which results in delivery to a desired location in the subject.

The compositions described herein may be administered with additional compositions to prolong stability and activity of the compositions, or in combination with other therapeutic drugs.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation or limitations which is not specifically disclosed herein.

This disclosure provides methods of making the compounds and compositions of the invention. The compounds and compositions can be prepared by any of the applicable techniques described herein, optionally in combination with standard techniques of organic synthesis. Many techniques such as etherification and esterification are well known in the art. However, many of these techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6; as well as standard organic reference texts such as March's Advanced Organic Chemistry: Reactions, Mechanisms, and Structure, 5th Ed., by M. B. Smith and J. March (John Wiley & Sons, New York, 2001); Comprehensive Organic Synthesis. Selectivity, Strategy & Efficiency in Modern Organic Chemistry. In 9 Volumes, Barry M. Trost, Editor-in-Chief (Pergamon Press, New York, 1993 printing); Advanced Organic Chemistry, Part B: Reactions and Synthesis, Second Edition, Cary and Sundberg (1983).

The formulas and compounds described herein can be modified using protecting groups. Suitable amino and carboxy protecting groups are known to those skilled in the art (see for example, Protecting Groups in Organic Synthesis, Second Edition, Greene, T. W., and Wutz, P. G. M., John Wiley & Sons, New York, and references cited therein; Philip J. Kocienski; Protecting Groups (Georg Thieme Verlag Stuttgart, New York, 1994), and references cited therein); and Comprehensive Organic Transformations, Larock, R. C., Second Edition, John Wiley & Sons, New York (1999), and referenced cited therein.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described hereinabove. The permissible substituents can be one or more and the same or different for appropriate organic compounds. For purposes of this disclosure, the heteroatoms, such as nitrogen, may have hydrogen substituents and/or any permissible substituents of organic compounds described herein which satisfy the valencies of the heteroatoms.

For example, the terms “substituted”, “substitution”, or “substituent” is intended to indicate that one or more (for example., 1-20 in various embodiments, 1-10 in other embodiments, 1, 2, 3, 4, or 5; in some embodiments 1, 2, or 3; and in other embodiments 1 or 2) hydrogens on the group indicated in the expression using “substituted” (or “substituent”) is replaced with a selection from the indicated group(s), or with a suitable group known to those of skill in the art, provided that the indicated atom's normal valency is not exceeded, and that the substitution results in a stable compound, e.g., which does not spontaneously undergo transformation such as by rearrangement, cyclization, elimination, etc. Suitable indicated groups include, e.g., alkyl, alkenyl, alkynyl, alkoxy, halo, haloalkyl, hydroxy, hydroxyalkyl, aryl, heteroaryl, heterocycle, cycloalkyl, alkanoyl, alkoxycarbonyl, amino, alkylamino, dialkylamino, trifluoromethylthio, difluoromethyl, acylamino, nitro, trifluoromethyl, trifluoromethoxy, carboxy, carboxyalkyl, keto, thioxo, alkylthio, alkylsulfinyl, alkylsulfonyl, and cyano. Additionally, non-limiting examples of substituents that can be bonded to a substituted carbon (or other) atom include F, Cl, Br, I, OR′, OC(O)N(R′)₂, CN, CF₃, OCF₃, R′, O, S, C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂NHC(O)R′, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further substituted. When a substituent is monovalent, such as, for example, F or Cl, it is bonded to the atom it is substituting by a single bond. When a substituent is more than monovalent, such as O, which is divalent, it can be bonded to the atom it is substituting by more than one bond, i.e., a divalent substituent is bonded by a double bond; for example, a C substituted with O forms a carbonyl group, C═O, wherein the C and the O are double bonded. Alternatively, a divalent substituent such as O, S, C(O), S(O), or S(O)₂ can be connected by two single bonds to two different carbon atoms. For example, O, a divalent substituent, can be bonded to each of two adjacent carbon atoms to provide an epoxide group, or the O can form a bridging ether group between adjacent or non-adjacent carbon atoms, for example bridging the 1,4-carbons of a cyclohexyl group to form a [2.2.1]-oxabicyclo system. Further, any substituent can be bonded to a carbon or other atom by a linker, such as (CH₂)ₙ or (CR′₂)ₙ wherein n is 1, 2, 3, or more, and each R′ is independently selected.

The term “halo” or “halide” refers to fluoro, chloro, bromo, or iodo. Similarly, the term “halogen” refers to fluorine, chlorine, bromine, and iodine.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof Such as racemic mixtures, form part of the present invention. Many organic compounds exist in optically active forms, i.e., they have the ability to rotate the plane of plane-polarized light. In describing an optically active compound, the prefixes D and L, or Rand S. are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate (defined below), which may occur where there has been no stereoselection or stereospecificity in a chemical reaction or process.

The terms “racemic mixture” and “racemate” refer to an equimolar mixture of two enantiomeric species, devoid of optical activity.

The term “enantiomerically enriched” (“ee”) as used herein refers to mixtures that have one enantiomer present to a greater extent than another. Reactions that provide one enantiomer present to a greater extent than another would therefore be “enantioselective” (or demonstrate “enantioselectivity”). In one embodiment of the invention, the term “enantiomerically enriched” refers to a mixture having at least about 2% ee; in another embodiment of the invention, the term “enantiomerically enriched” refers to a mixture having at least about 5% ee; in another embodiment of the invention, the term “enantiomerically enriched” refers to a mixture having at least about 20%; in another embodiment of the invention, the term “enantiomerically enriched” refers to a mixture having at least about 50%; in another embodiment of the invention, the term “enantiomerically enriched” refers to a mixture having at least about 80%; in another embodiment of the invention, the term “enantiomerically enriched” refers to a mixture having at least about 90%; in another embodiment of the invention, the term “enantiomerically enriched” refers to a mixture having at least about 95%; in another embodiment of the invention, the term “enantiomerically enriched” refers to a mixture having at least about 98%; in another embodiment of the invention, the term “enantiomerically enriched” refers to a mixture having at least about 99%. The term “enantiomerically enriched” includes enantiomerically pure mixtures which are mixtures that are substantially free of the species of the opposite optical activity or one enantiomer is present in very low quantities, for example, 0.01%, 0.001% or 0.0001%.

The term “heteroatom” is art-recognized and refers to an atom of any element other than carbon or hydrogen. Illustrative heteroatoms include boron, nitrogen, oxygen, phosphorus, sulfur and selenium.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In preferred embodiments, a straight chain or branched chain alkyl has 30 or fewer carbon atoms in its backbone (e.g., C₁-C₃₀ for straight chain, C₃-C₃₀ for branched chain), and more preferably 20 or fewer. For example, (C₁-C₆)alkyl. Likewise, preferred cycloalkyls have from 3-10 carbon atoms in their ring structure, and more preferably have 5, 6 or 7 carbons in the ring structure.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths but with at least two carbon atoms. Preferred alkyl groups are lower alkyls. In preferred embodiments, a substituent designated herein as alkyl is a lower alkyl.

The term “aralkyl”, as used herein, means an aryl group, as defined herein, appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

The term “alkoxy” means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxycarbonyl” means an alkoxy group, as defined herein, appended to the parent molecular moiety through a carbonyl group, represented by —C(═O)—, as defined herein. Representative examples of alkoxycarbonyl include, but are not limited to, methoxycarbonyl, ethoxycarbonyl, and tert-butoxycarbonyl.

The term “carboxy” as used herein, means a —CO₂H group.

The term “alkylthio” as used herein, means an alkyl group, as defined herein, appended to the parent molecular moiety through a sulfur atom. Representative examples of alkylthio include, but are not limited, methylthio, ethylthio, tert-butylthio, and hexylthio. The terms “arylthio,” “alkenylthio” and “arylakylthio,” for example, are likewise defined.

The term “amido” as used herein, means —NHC(═O)—, wherein the amido group is bound to the parent molecular moiety through the nitrogen. Examples of amido include alkylamido such as CH₃C(═O)N(H)— and CH₃CH₂C(═O)N(H)—.

The term “aryl” as used herein includes 5-, 6- and 7-membered aromatic groups that may include from zero to four heteroatoms, for example, benzene, naphthalene, anthracene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with such substituents as described above, for example, halogen, azide, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls.

The abbreviations Me, Et, Ph, Tf, Nf, Ts, Ms, and dba represent methyl, ethyl, phenyl, trifluoromethanesulfonyl, nonafluorobutanesulfonyl, p-toluenesulfonyl, methanesulfonyl, and dibenzylideneacetone, respectively. Also, “DCM” stands for dichloromethane; “rt” stands for room temperature, and may mean about 20° C., about 21° C., about 22° C., about 23° C., about 24° C., about 25° C., or about 26° C.; “THF” stands for tetrahydrofuran; “BINAP” stands for 2,2′-bis(diphenylphosphino)-1,1′-binaphthyl; “dppf” stands for 1,1′-bis(diphenylphosphino)ferrocene; “dppb” stands for 1,4-bis(diphenylphosphinobutane; “dppp” stands for 1,3-bis(diphenylphosphino)propane; “dppe” stands for 1,2-bis(diphenylphosphino)ethane. A more comprehensive list of the abbreviations utilized by organic chemists of ordinary skill in the art appears in the first issue of each volume of the Journal of Organic Chemistry; this list is typically presented in a table entitled Standard List of Abbreviations. The abbreviations contained in said list, and all abbreviations utilized by organic chemists of ordinary skill in the art are hereby incorporated by reference.

The terms ortho, meta and para apply to 1,2-, 1,3- and 1,4-disubstituted benzenes, respectively. For example, the names 1,2-dimethylbenzene and ortho-dimethylbenzene are synonymous.

The terms “heterocyclyl”, “heterocycloalkyl”, or “heterocyclic group” refer to 3- to 10-membered ring structures, more preferably 3- to 7-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with one or more substituents, for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “heteroatom” as used herein means an atom of any element other than carbon or hydrogen. Preferred heteroatoms are nitrogen, oxygen, sulfur and phosphorous.

As used herein, the term “nitro” means —NO₂; the term “halogen” or “halo” designates —F, —Cl, —Br or —I; the term “sulfhydryl” means —SH; the term “hydroxyl” means —OH; the term “sulfonyl” means —SO₂—; and the term “cyano” as used herein, means a —CN group.

The term “haloalkyl” means at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The terms “amine” and “amino” are art recognized and refer to both unsubstituted and substituted amines, e.g., a moiety that can be represented by the general formula:

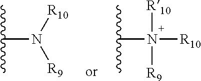

wherein R₉, R₁₀ and R′₁₀ each independently represent a hydrogen, an alkyl, an alkenyl, —(CH₂)ₘ—R₈, or R₉ and R₁₀ taken together with the N atom to which they are attached complete a heterocycle having from 4 to 8 atoms in the ring structure; R₈ represents an aryl, a cycloalkyl, a cycloalkenyl, a heterocycle or a polycycle; and m is zero or an integer in the range of 1 to 8. In preferred embodiments, only one of R₉ or R₁₀ can be a carbonyl, e.g., R₉, R₁₀ and the nitrogen together do not form an imide. In even more preferred embodiments, R₉ and R₁₀ (and optionally R′₁₀) each independently represent a hydrogen, an alkyl, an alkenyl, or —(CH₂)ₘ—R₈. Thus, the term “alkylamine” as used herein means an amine group, as defined above, having a substituted or unsubstituted alkyl attached thereto, i.e., at least one of R₉ and R₁₀ is an alkyl group.

The definition of each expression, e.g., alkyl, m, n, and the like, when it occurs more than once in any structure, is intended to be independent of its definition elsewhere in the same structure.

The terms triflyl (-Tf), tosyl (-Ts), mesyl (-Ms), and nonaflyl are art-recognized and refer to trifluoromethanesulfonyl, p-toluenesulfonyl, methanesulfonyl, and nonafluorobutanesulfonyl groups, respectively. The terms triflate (-OTf), tosylate (-OTs), mesylate (-OMs), and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The phrase “protecting group” as used herein means temporary modifications of a potentially reactive functional group which protect it from undesired chemical transformations. Examples of such protecting groups include silyl ethers of alcohols, and acetals and ketals of aldehydes and ketones, respectively. In embodiments of the disclosure, a carboxylate protecting group masks a carboxylic acid as an ester. In certain other embodiments, an amide is protected by an amide protecting group, masking the —NH₂ of the amide as, for example, —NH(alkyl), or —N(alkyl)₂. The field of protecting group chemistry has been reviewed (Greene, T. W.; Wuts, P. G. M. Protective Groups in Organic Synthesis, 2^{nd} ed.; Wiley: New York, 1991).

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

As used herein, the term “rotatable bonds” as used herein is a count of single bonds, not in a ring, bound to a nonterminal heavy atom. Excluded from the count are C—N amide bonds because of their high rotational energy barrier. Rotatable bonds are abbreviated as “RB” herein.

### Exemplary Compounds

The compounds disclosed herein exclude (E)-N-methyl-N-((3-methylbenzofuran-2-yl)methyl)-3-(7-oxo-5,6,7,8-tetrahydro-1,8-naphthyridin-3-yl)acrylamide (Debio-1452), and (R)- and (S)-enantiomers of (E)-3-(6-amino-7-oxo-5,6,7,8-tetrahydro-1,8-naphthyridin-3-yl)-N-methyl-N-((3-methylbenzofuran-2-yl)methyl)acrylamide (Debio-1452-NH2).

This disclosure provides a compound of Formula I or II:

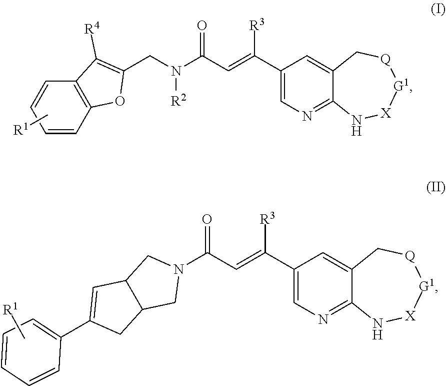

or a salt thereof; wherein

G¹ is —CH(C₀-C₆)alkyl-NH₂, CH₂, CH(C₁-C₆)alkyl, —CH(C₃-C₆)cycloalkyl-NH₂, —CHN(H)(C₂-C₆)alkyl-NH₂, or G₂;

G² is

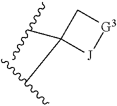

wherein G³ is NH, —CH(C₀-C₆)alkyl-NH₂, or —CH₂CH(C₀-C₆)alkyl-NH₂;
J is —(CH₂)ₘ—; and m is 1, 2, or 3;

Q is CHR^{a}, NH, —NHCH₂—, —CH₂CH₂—, —C(═O)N—, or absent, wherein R^{a} is H or halo;

X is C═O or CH₂;

R¹ is H, halo, —(C₁-C₆)alkyl, or —(C₁-C₆)alkyl-NH₂;

R² is —(C₁-C₆)alkyl, H, or —(C₁-C₆)alkyl-NH₂;

R³ is H or —(C₁-C₆)alkyl-NH₂; and

R⁴ is methyl, ethyl, propyl, butyl, pentyl, hexyl, or —(C₃-C₆)cycloalkyl;

wherein for Formula I, R² and R⁴ taken together optionally form a heterocycle.

In various embodiments, the moiety (C₁-C₆), or the moiety (C₀-C₆) for C₁-C₆, is optionally substituted, optionally branched, optionally unsaturated, optionally cyclic for C₃-C₆, optionally interrupted with a heteroatom, optionally interrupted with a carbocycle, or a combination thereof. In various embodiments, at least one of G¹, R¹, R², or R³ comprises an amine moiety. In various embodiments, each NH and/or NH₂ moiety is optionally substituted with —(C₁-C₆)alkyl or a protecting group (e.g., tBoc or acetate) for each H on the nitrogen atom.

In some embodiments, the methylene moiety (e.g., —CH₂—) between Q and the pyridine ring in any one of the formulas disclosed herein, for example Formulas I-IV, is C═O or —CHR^{v}— wherein R^{v} is —(C₀-C₆)alkyl-NH₂, —(C₃-C₆)cycloalkyl-NH₂, —N(H)(C₂-C₆)alkyl-NH₂, or a halo such as F; or forms an endocyclic or exocyclic double bond. In some embodiments, R¹ is also —(C₁-C₆)alkyl or —(C₃-C₆)cycloalkyl wherein said alkyl or said cycloalkyl is saturated or unsaturated and optionally substituted with an amine such as NH₂. In some embodiments, the NH moiety of NH₂ moiety is acylated, alkylated, or otherwise protected with a protecting group such as t-Boc.

In some embodiments, G¹ is G². In other embodiments, the compound is represented by Formula I, Q is absent, and R² or R³ is —(C₁-C₆)alkyl-NH₂. In various embodiments, R 1 is fluoro.

In additional embodiments, the compound is represented by Formula IA:

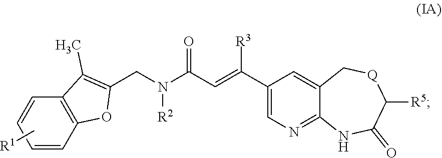

wherein

R⁵ is —(C₀-C₆)alkyl-NH₂ or —NH(C₂-C₆)alkyl-NH₂.

In various other embodiments, Q is CH₂ or CHF. In some embodiments, Q is CH₂, R¹ is H or fluoro, R² is methyl, R³ is H, and R⁵ is NH₂ or CH₂NH₂. In some other embodiments, Q is absent, R¹ is fluoro, R² is methyl, R³ is H, and R⁵ is —NHCH₂CH₂NH₂ or —NHCH₂CH₂CH₂NH₂.

In various embodiments, the compound is:

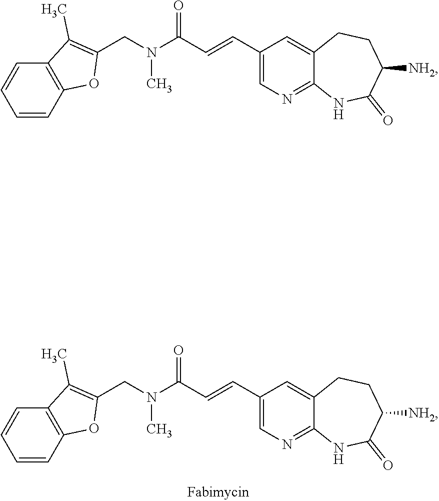 [SMILES: Cc1c(CN(C)C(=O)/C=C/c2cnc3c(c2)CC[C@@H](N)C(=O)N3)oc2ccccc12 ; Cc1c(CN(C)C(=O)/C=C/c2cnc3c(c2)CC[C@H](N)C(=O)N3)oc2ccccc12]

or a salt thereof.

In some embodiments, the compound is (R,E)-3-(7-amino-8-oxo-6,7,8,9-tetrahydro-5H-pyrido[2,3]azepin-3-yl)-N-methyl-N-((3-methylbenzofuran-2-yl)methyl)acrylamide; (S,E)-3-(7-amino-8-oxo-6,7,8,9-tetrahydro-5H-pyrido[2,3-b]azepin-3-yl)-N-methyl-N-((3-methylbenzofuran-2-yl)methyl)acrylamide; or a salt thereof.

In further embodiments, the compound is represented by Formula IB:

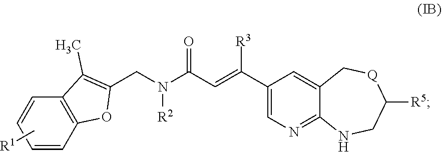

wherein

Q is NH, or —C(═O)N— wherein the carbonyl moiety of the amide group is at the position alpha to the tertiary carbon that is substituted with R⁵; and R⁵ is —(C₀-C₆)alkyl-NH₂ or —NH(C₂-C₆)alkyl-NH₂.

In other additional embodiments, the compound is represented by Formula IC:

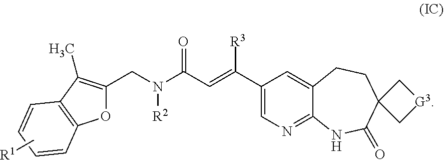

In various embodiments, G³ is NH.

In yet other additional embodiments, the compound is represented by Formula ID:

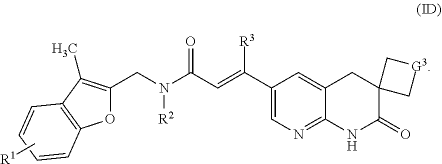

In various other embodiments, G³ is NH. In various additional embodiments, G³ is —CH(C₀-C₆)alkyl-NH₂. In some embodiments, G³ is NH, —CHNH₂, or —CHCH₂NH₂, R¹ is fluoro, R² is methyl, and R³ is H.

In other embodiments, the compound is:

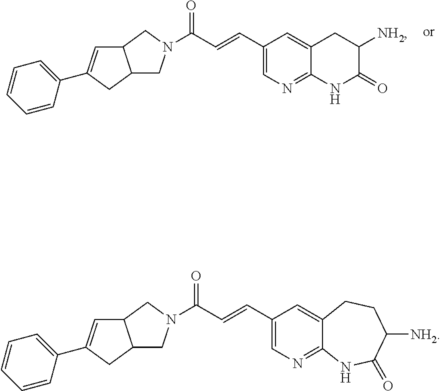 [SMILES: NC1Cc2cc(/C=C/C(=O)N3CC4C=C(c5ccccc5)CC4C3)cnc2NC1=O ; "or" ; NC1CCc2cc(/C=C/C(=O)N3CC4C=C(c5ccccc5)CC4C3)cnc2NC1=O]

In yet other embodiments, the compound is:

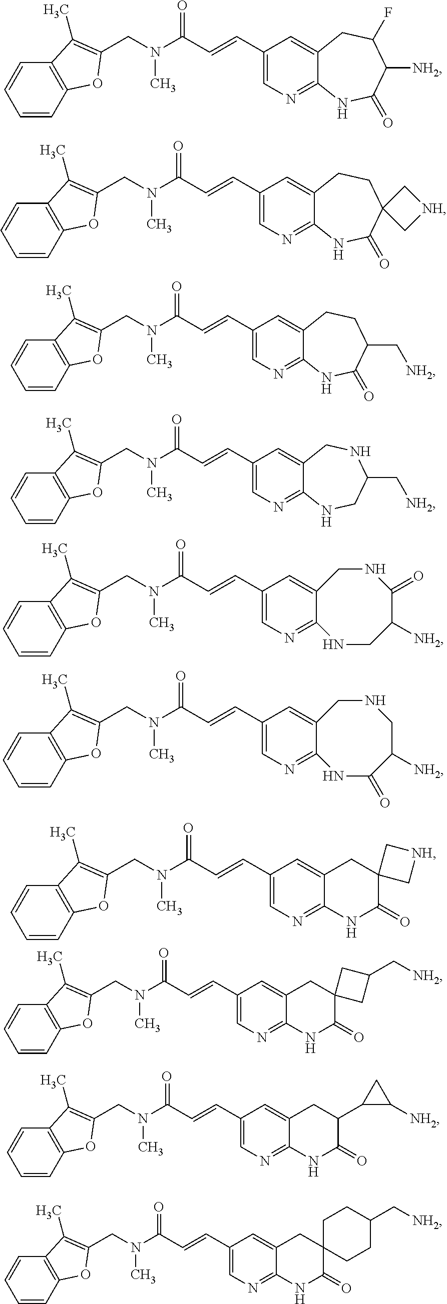

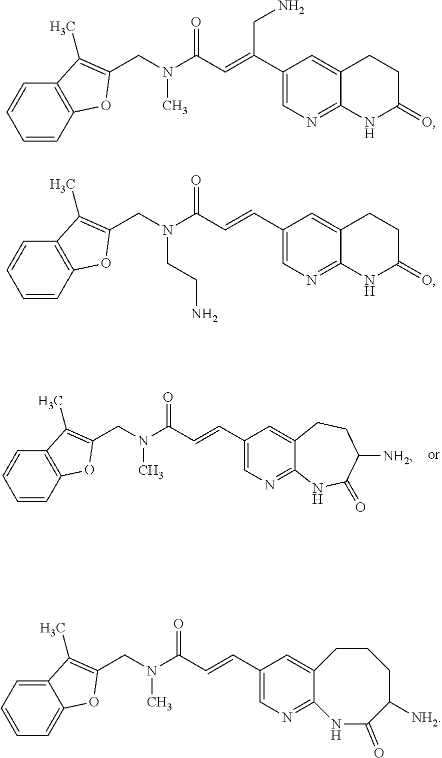 [SMILES: Cc1c(CN(C)C(=O)/C=C(\CN)c2cnc3c(c2)CCC(=O)N3)oc2ccccc12 ; Cc1c(CN(CCN)C(=O)/C=C/c2cnc3c(c2)CCC(=O)N3)oc2ccccc12 ; Cc1c(CN(C)C(=O)/C=C/c2cnc3c(c2)CCC(N)C(=O)N3)oc2ccccc12 ; Cc1c(CN(C)C(=O)/C=C/c2cnc3c(c2)CCCC(N)C(=O)N3)oc2ccccc12]

This disclosure also provides a compound of Formula III or IIIB:

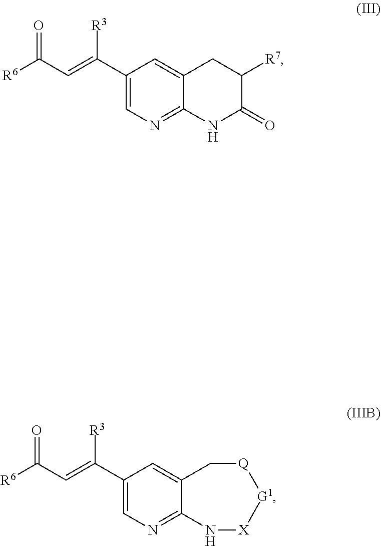

or a salt thereof; wherein

R⁶ is

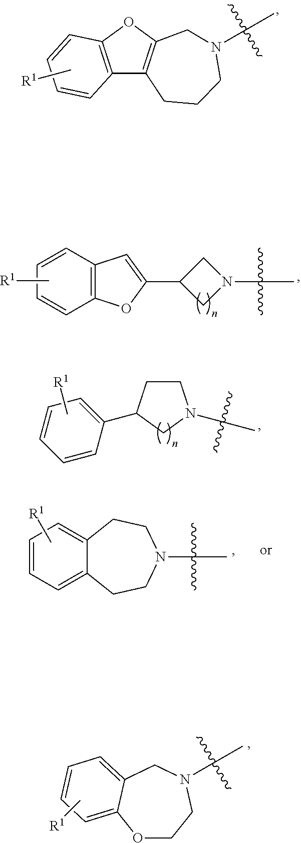

wherein n is 1-4 and the other variables are defined above. In some embodiments, R¹ is H or halo, R³ is H or —(C₁-C₆)alkyl, and R⁷ is H or —(C₀-C₆)alkyl-NH₂.

In some embodiments, the compound is:

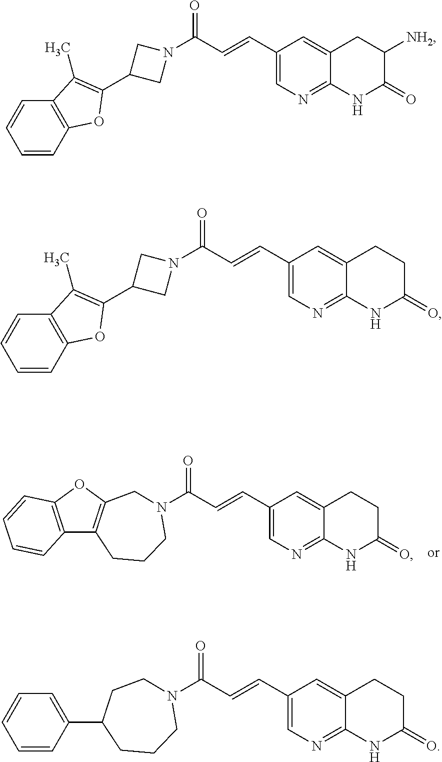 [SMILES: Cc1c(C2CN(C(=O)/C=C/c3cnc4c(c3)CC(N)C(=O)N4)C2)oc2ccccc12 ; Cc1c(C2CN(C(=O)/C=C/c3cnc4c(c3)CCC(=O)N4)C2)oc2ccccc12 ; O=C1CCc2cc(/C=C/C(=O)N3CCCc4c(oc5ccccc45)C3)cnc2N1 ; "or" ; O=C1CCc2cc(/C=C/C(=O)N3CCCC(c4ccccc4)CC3)cnc2N1]

This disclosure further provides a compound of Formula IV:

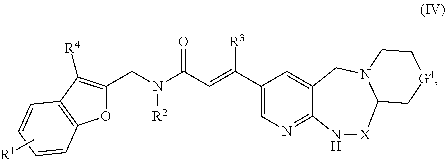

or a salt thereof; wherein

G⁴ is O, NH, CH₂, —CH(C₀-C₆)alkyl-NH₂, —N(C═O)(C₁-C₆)alkyl-NH₂;

X is C═O or CH₂;

R¹ is H or halo;

R² is H, —(C₁-C₆)alkyl, or —(C₁-C₆)alkyl-NH₂;

R³ is H or —(C₁-C₆)alkyl-NH₂; and

R⁴ is methyl, ethyl, or propyl;

wherein at least one of G⁴, R², or R³ comprises an amine moiety.

In some embodiments, the compound is:

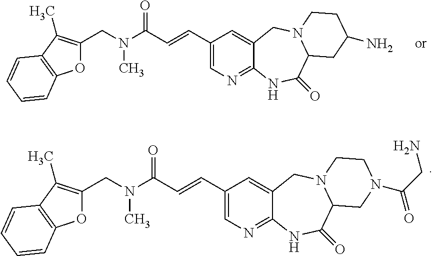 [SMILES: Cc1c(CN(C)C(=O)/C=C/c2cnc3c(c2)CN2CCC(N)CC2C(=O)N3)oc2ccccc12 ; Cc1c(CN(C)C(=O)/C=C/c2cnc3c(c2)CN2CCN(C(=O)CN)CC2C(=O)N3)oc2ccccc12]

This disclosure provides a pharmaceutical composition comprising a compound disclosed herein and a pharmaceutically acceptable excipient.

This disclosure also provides a method of antimicrobial treatment comprising administering to a subject in need thereof a therapeutically effective amount of a compound disclosed herein or a pharmaceutically acceptable salt thereof, thereby killing or inhibiting the growth of at least a portion of a plurality of microorganisms in the subject.

In various embodiments, the microorganism is a. Gram-negative bacterium. In various embodiments, the disclosed method is a method for treating a urinary tract infection (UTI). In various embodiments, the microorganisms are Escherichia Coli. In various embodiments, the compound is fabimycin.

In other embodiments, the microorganism is Acinetobacter, anthrax-causing bacteria, Bacilli, Bordetella, Borrelia, botulism-causing bacteria, Brucella, Burkholderia, Campylobacter, Chlamydia, cholera-causing bacteria, Clostridium, Gonococcus, Corynebacterium, diphtheria-causing bacteria, Enterobacter, Enterococcus, Erwinia, Escherichia, Francisella, Haemophilus, Heliobacter, Klebsiella, Legionella, Leptospira, leptospirosis-causing bacteria, Listeria, Lyme's disease-causing bacteria, meningococcus, Mycobacterium, Mycoplasma, Neisseria, Pasteurella, Pelobacter, plague-causing bacteria, Pneumonococcus, Proteus, Pseudomonas, Rickettsia, Salmonella, Serratia, Shigella, Staphylococcus, Streptococcus, tetanus, Treponema, Vibrio, Yersinia and Xanthomonas, or a combination thereof.

### Exemplary Methods

In certain embodiments, the compounds disclosed herein accumulate in Gram-negative bacteria.

In certain embodiments, the compounds disclosed herein traverse a porin.

In certain embodiments, provided herein is a method of antimicrobial treatment, comprising, administering to a subject in need thereof a therapeutically effective amount of a compound disclosed herein, or a pharmaceutically acceptable salt thereof, thereby killing or inhibiting the growth of at least a portion of a plurality of microorganisms in the subject.

In certain embodiments, the compound is a compound of any one of Formulas I, IA, IB, IC, ID, II, III, IIIB, or IV.

In certain embodiments, provided herein is a method of antimicrobial treatment, comprising providing a sample comprising a plurality of microorganisms; and contacting the sample with a compound disclosed herein; thereby killing or inhibiting the growth of at least a portion of the plurality of microorganisms in the sample.

In certain embodiments of the methods of antimicrobial treatment disclosed herein, at least a portion of the plurality of microorganisms is killed. In certain embodiments, the growth of at least a portion of the plurality of microorganisms is inhibited.

In certain embodiments, the microorganism is a bacterium, a virus, a fungus, or a parasite. In certain embodiments, the microorganism is drug resistant, such as antibiotic resistant. In certain embodiments, the microorganism is multi-drug resistant.

In certain embodiments, the microorganism is a bacterium. In certain embodiments, the microorganism is a Gram-negative bacterium. In certain embodiments, the microorganism is a Gram-positive bacterium. In certain embodiments, for example, the microorganism is at least one bacterium selected from the group consisting of Acinetobacter, anthrax-causing bacteria, Bacilli, Bordetella, Borrelia, botulism, Brucella, Burkholderia, Campylobacter, Chiamyclia, cholera-causing bacteria, Clostridium, Conococcus, Corynebacterium, diphtheria-causing bacteria, Enterobacter, Enterococcus, Erwinia, Escherichia, Francisella, Haemophilus, Heliobacter, Klebsiella, Legionella, Leptospira, leptospirosis-causing bacteria, Listeria, Lyme's disease-causing bacteria, meningococcus, Mycobacterium, Mycoplasma, Neisseria, Pasteurella, Pelobacter, plague-causing bacteria, Pneumonococcus, Proteus, Pseudomonas, Rickettsia, Salmonella, Serratia, Staphylococcus, Streptococcus, tetanus-causing bacteria, Treponema, Vibrio, Yersinia and Xanthomonas. In certain embodiments, the microorganism is at least one bacterium selected from the group consisting of Acinetobacter baumannii, Escherichia coli, Enterobacter cloacae, Klebsiella pneumoniae, Pseudomorms aeruginosa, and Staphylococcus aureus. In certain embodiments, the microorganism is methicillin-resistant Staphylococcus aureus (MRSA). In certain embodiments, the microorganism is Pseudomonas aeruginosa.

In certain embodiments, for example, the microorganism is at least one virus selected from Adenoviridae, Papillomaviridae, Polymaviridae, Herpesviridae, Poxviridae, Hepadnaviridae, Parvoviridae, Astroviridae, Caliciviridae, Picornaviridae, Coronoviridae, Flaviviridae, Retroviridae, Togaviridae, Arenaviridae, Bunyaviridae, Orthomyxoviridae, Paramyxoviridae, Rhabdoviridae, and Reoviridae. In certain embodiments, the virus may be arboviral encephalitis virus, adenovirus, herpes simplex type I, herpes simplex type 2, Varicella-zoster virus, Epstein-barr virus, cytomegalovirus, herpesvirus type 8, papillomavirus, BK virus, coronavirus, echovirus, JC virus, smallpox, Hepatitis B, bocavims, parvovirus B19, astrovirus, Norwalk virus, coxsackievirus, Hepatitis A, poliovirus, rhinovirus, severe acute respiratory syndrome virus, Hepatitis C, yellow fever, dengue virus, West Nile virus, rubella, Hepatitis E, human immunodeficiency virus (HIV), human T-cell lymphotropic virus (HTLV), influenza, guanarito virus, Junin virus, Lassa virus, Machupo virus, Sabia virus, Crimean-Congo hemorrhagic fever virus, ebola virus, Marburg virus, measles virus, molluscum virus, mumps virus, parainfluenza, respiratory syncytial virus, human metapneumovirus, Hendra virus, Nipah virus, rabies, Hepatitis D, rotavirus, orhivirus, coltivirus, vaccinia virus, and Banna virus.

In certain embodiments, for example, the microorganism is at least one fungus selected from Aspergillus (fumigatus, niger, etc.), Basidiobolus (ranarum, etc.), Blastomyces dermatitidis, Candida (albicans, krusei, glabrata, tropicalis, etc.), Coccidioides immitis, Cryptococcus (neoformans, etc.), eurnycetoma, Epidermophyton (floccosum, etc.), Histoplasma capsulatum, Hortaea werneckii, Lacazia loboi, Microsproum (audouinii, nanum etc.), Mucorales (mucor, absidia, rhizophus), Paracoccidioides brasiliensis, Rhinosporidium seeberi, Sporothrix schenkii, and Trichophyton (schoeleinii, mentagrophytes, rubrum, verrucosum, etc.).

In certain embodiments, for example, the microorganism is at least one parasite selected from Acanthamoeba, Babesia microti, Balantidium coli, Entamoeba hystolytica, Giardia lamblia, Cryptosporidium muris, Trvanosomatida gambiense, Ttypanosomatida rhodesiense, Trypanosoma brucei, Trypanosoma cruzi, Leishmania mexicana, Leishmania braziliensis, Leishmania tropica, Leishmania donovani, Toxoplasma gondii, Plasmodium vivax, Plasmodium ovale, Plasmodium malariae Plasmodium falciparum, Pneumocystis carinii, Trichomonas vaginalis, Histomonas meleagridis, Secementea, Trichuris trichiura, Ascaris lumbricoides, Enterobius vermicularis, Ancylostoma duodenale, Naegleria fowleri, Necator americanus, Nippostrongylus brasiliensis, Strongyloides stercoralis, Wuchereria banerofti, Dracunculus medinensis, blood flukes, liver flukes, intestinal flukes, lung flukes, Schistosoma mansoni, Schistosoma haematobium, Schistosoma japonicum, Fasciola hepatica, Fasciola gigantica, Heterophyes heterophyes, and Paragonimus westermani.

In certain embodiments, the subject is a mammal or reptile. In certain embodiments, the mammal is a primate, feline, canine, rodent, ovine, or bovine. In certain embodiments, the mammal is a human In other embodiments, the subject is a vertebrate or invertebrate. In other embodiments, the subject is a fish, amphibian, or bird.

### Results and Discussion

Identification of fabimycin and its activity against drug-resistant Gram-negative pathogens. Guidance by the eNTRy rules enabled Debio-1452-NH3 to be identified through synthesis and evaluation of just a handful of compounds (Ann N Y Acad Sci 2019, 1435 (1), 18). However, to advance as a lead candidate its therapeutic window would need to be widened, as efficacy of Debio-1452-NH3 in murine infection models with Gram-negative pathogens was observed near the maximal tolerated dose (MTD). Thus, an objective was set to identify next-generation versions more potent against Gram-negative clinical isolates and with greater tolerability in vivo, with the expectation that such compounds could then be efficacious even in challenging models and those of high translational relevance, such as a UTI model.

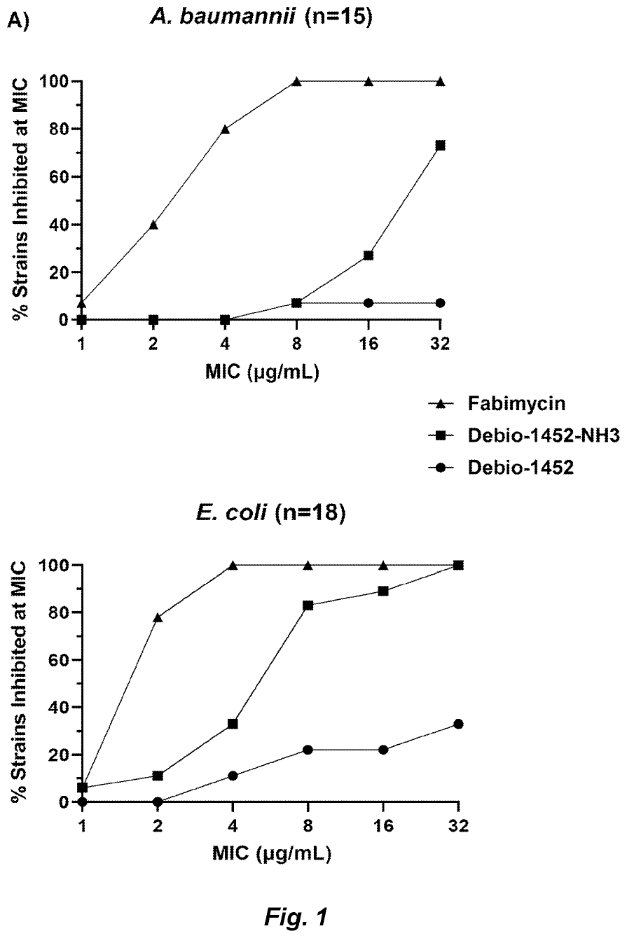

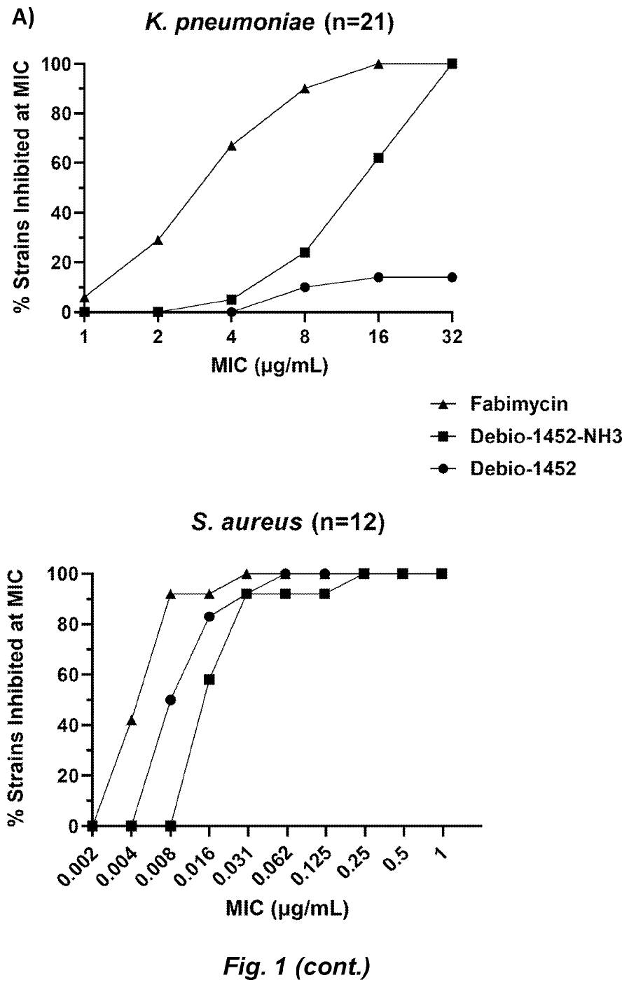

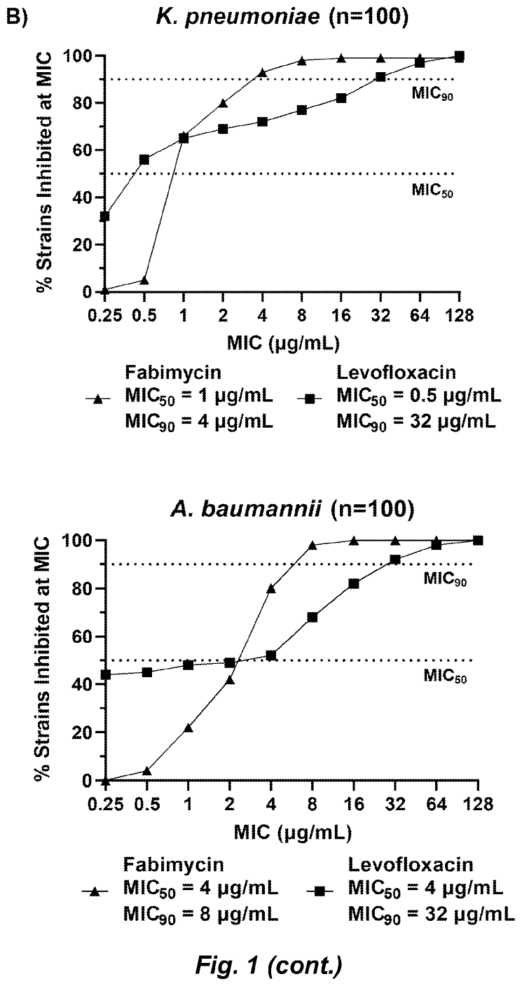

Our critical path for advancement included synthesis of compounds and subsequent evaluation of antibacterial activity against Gram-negative clinical isolates to prioritize leads followed by evaluation of toxicity, pharmacokinetics, and ultimately efficacy in mouse infection models. Compound design was guided by the co-crystal structure of Debio-1452 with FabI (from S. aureus) and the established SAR for this compound class, both of which pointed to the immutability of N-methyl acrylamide (E configuration), and the H-bond donor/acceptor pair on the naphthyridinone which interacts with key amino acid residues within the FabI active site (FIG. 1B). In contrast, the 3-position of the naphthyridinone ring (adjacent to the carbonyl) was judged to be highly solvent-exposed; in addition, other ring systems were considered as replacements for the benzopyran (FIG. 1B). Embedded in this SAR was the necessity for proper placement of a positively-charged amine functionality to facilitate accumulation in Gram-negative bacteria but not disrupt target engagement. Given the x-ray and SAR data, and lessons learned from previous identification of Debio-1452-NH3, priority compounds were envisioned with a variety of amines and ring systems proximal to the carbonyl of the naphthyridinone.

To construct the target compounds a modular synthesis was implemented where Heck coupling of acrylic amide 23 (Scheme 1) with brominated N-Boc functionalized naphthyridinone rings provided, after deprotection, the final compounds (Table 1). Bromo-naphthyridinones were designed to give final compounds that maintained the necessary N/NH arrangement of the oxotetrahydronapthyridine ring system and contained the requisite amine, while altering ring size and shape to quickly interrogate the solvent-exposed region of the scaffold adjacent to the lactam carbonyl. Specifically, a series with a four-membered ring spirocycles (5-7), six-membered ring spirocyclies (8, 9), and ring expanded napthyridinones (11-13) were constructed.

TABLE 1
Debio-1452 analogue synthesis and antibacterial activity in MIC (μg/mL). The general synthetic
route utilized to synthesize amine-containing ring systems, and their antimicrobial activities in Gram-
positive and Gram-negative bacteria. (*) indicates a racemic mixture.

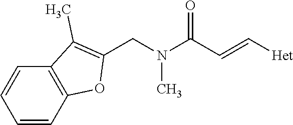

where Het is:

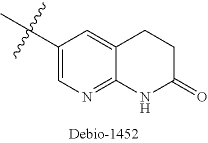 [SMILES: *c1cnc2c(c1)CCC(=O)N2]

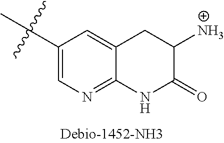 [SMILES: *c1cnc2c(c1)CC([NH3+])C(=O)N2]

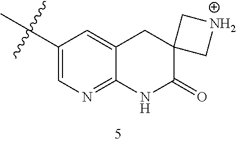 [SMILES: *c1cnc2c(c1)CC1(C[NH2+]C1)C(=O)N2]

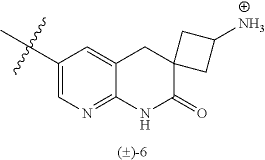 [SMILES: *c1cnc2c(c1)CC1(CC([NH3+])C1)C(=O)N2]

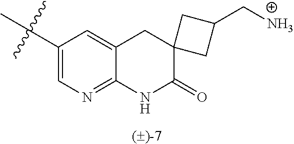 [SMILES: *c1cnc2c(c1)CC1(CC(C[NH3+])C1)C(=O)N2]

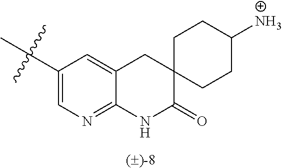 [SMILES: *c1cnc2c(c1)CC1(CCC([NH3+])CC1)C(=O)N2]

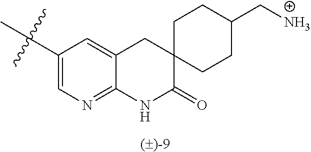 [SMILES: *c1cnc2c(c1)CC1(CCC(C[NH3+])CC1)C(=O)N2]

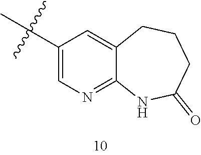 [SMILES: *c1cnc2c(c1)CCCC(=O)N2]

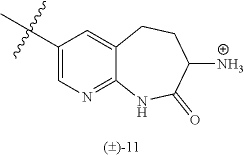 [SMILES: *c1cnc2c(c1)CCC([NH3+])C(=O)N2]

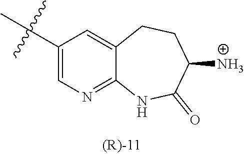 [SMILES: *c1cnc2c(c1)CC[C@@H]([NH3+])C(=O)N2]

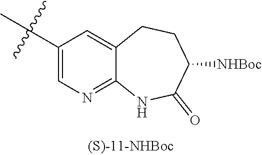 [SMILES: *c1cnc2c(c1)CC[C@H](NC(=O)OC(C)(C)C)C(=O)N2]

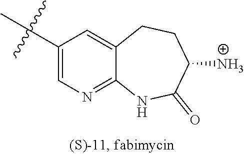 [SMILES: *c1cnc2c(c1)CC[C@H]([NH3+])C(=O)N2]

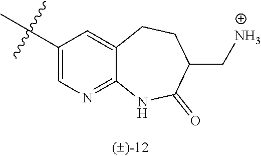 [SMILES: *c1cnc2c(c1)CCC(C[NH3+])C(=O)N2]

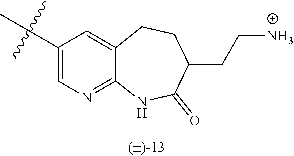 [SMILES: *c1cnc2c(c1)CCC(CC[NH3+])C(=O)N2]

| | Reference Strains | | | E. coli | | K. pneumoniae | | | | A. baumanni | | | |
|---|---|---|---|---|---|---|---|---|---|---|---|---|---|
| | S. aureus | E. coli | E. coli | AR | AR | AR | AR | AR | BAA | AR | AR | AR | AR |
| Compound | 29213 | ΔtolC | MG1655 | 0085 | 0048 | 0066 | 0113 | 0560 | 2472 | 0033 | 0273 | 0299 | 0313 |
| Debio- | 0.008 | 0.062 | >32 | >32 | >32 | >32 | >32 | >32 | >32 | >32 | >32 | >32 | >32 |
| 1452 | | | | | | | | | | | | | |
| Debio- | 0.031 | 0.062 | 4 | 16 | 32 | 32 | 32 | 32 | 16 | 32 | 32 | 32 | 32 |
| 1452NH3 | | | | | | | | | | | | | |
| 5 | 0.016 | 0.031 | 4 | 4 | 8 | 8 | 16 | 16 | 8 | 8 | 8 | 16 | 16 |
| (±)-6 | 0.016 | 0.062 | 4 | 16 | 16 | 8 | 16 | 8 | 8 | 16 | 16 | 16 | 32 |
| (±)-7 | 0.031 | 0.125 | 16 | — | — | — | — | — | — | — | — | — | — |
| (±)-8 | 0.016 | 0.062 | 16 | — | — | — | — | — | — | — | — | — | — |
| (±)-9 | 0.031 | 0.062 | 16 | — | — | — | — | — | — | — | — | — | — |
| (±)-11 | 0.016 | 0.031 | 4 | 4 | 4 | 8 | 8 | 8 | 8 | 4 | 8 | 4 | 8 |
| (±)-12 | 0.031 | 0.125 | 8 | 8 | 16 | 32 | 32 | 16 | 32 | 16 | 32 | 32 | >32 |
| (±)-13 | 0.062 | 0.125 | 32 | — | — | — | — | — | — | — | — | — | — |
| (R)-11 | 0.125 | 0.5 | 64 | 128 | 128 | >128 | >128 | >128 | >128 | >128 | >128 | >128 | >128 |
| (S)-11 | 0.004 | 0.016 | 2 | 1 | 2 | 4 | 4 | 4 | 4 | 8* | 4 | 2* | 4* |
| 10 | 0.002 | 0.031 | 8 | 8 | 8 | 32 | 32 | >32 | 16 | 16 | 8 | 8 | 16 |
| (S)-11- | — | — | 8 | 8 | 16 | 64 | 32 | 32 | 32 | 64 | 16* | 4 | 64 |
| NHBoc | | | | | | | | | | | | | |

The compounds synthesized, shown in Table 1, were evaluated against Gram-positive and Gram-negative reference strains (S. aureus 29213 and E. coli MG1655, respectively), as well as an efflux deficient E. coli strain (ΔtolC, JW5503). While compounds (±)-7, (±)-8, (±)-9, and (±)-13 all had reduced antibacterial activity against E. coli MG1655 relative to Debio-1452-NH3, compounds 5, (±)-6, (±)-11, and (±)-12 maintained good activity (Table 1) and were thus further evaluated against Gram-negative clinical isolates. For this experiment a panel of ten clinical isolates were selected where Debio-1452-NH3 is only minimally active (MIC values of 16 or 32 μg/mL), including two E. coli, four K. pneumoneia, and four A. baumannii strains. As expected, Debio-1452 has no activity against this Gram-negative ‘challenge panel’, and Debio-1452-NH3 has only minimal activity (Table 1). Assessment of 5, (±)-6, (±)-11, and (±)-12 revealed more potent activity for all four compounds relative to Debio-1452-NH3, with the ϵ-caprolactam (±)-11 emerging as a promising lead, possessing superior activity against the clinical isolate challenge panel with all strains being inhibited at 8 μg/mL. Two derivatives were constructed where the ϵ-caprolactam was coupled to alternatives ring systems, substituting out the benzofuran, but neither of these compounds provided an improvement in activity.

The two enantiomers of (±)-11 were separated by chiral preparatory HPLC, and biological assessment revealed the (−) enantiomer to possess significantly greater antibacterial activity than the (+) enantiomer. X-ray crystallography studies, described later herein, were used to determine that the highly active (−) enantiomer possesses the S stereochemical configuration; as shown in Table 1, (S)-11, coined fabimycin, possesses outstanding activity against S. aureus and E. coli ΔtolC, and an MIC value of 2 μg/mL against E. coli MG1655, whereas (R)-11 is significantly less active.

To probe the influence of the amine on antibacterial activity and compound accumulation, two additional derivatives were synthesized: compound 10, with the expanded ring system but lacking the amine, and compound (S)-11-NHBoc, an acylated version of fabimycin. Both of these compounds lack a positive charge adjacent to the carbonyl. While both compounds maintain excellent inhibition of FabI, as judged by antimicrobial activity against S. aureus and E. coli ΔAtolC, and by biochemical inhibition of FabI, they have significantly diminished activity against Gram-negative clinical isolates (Table 1). These compounds were then assessed in a whole cell accumulation assay in E. coli, and consistent with the observed reduction in antibacterial activity, 10 and (S)-11-NHBoc showed no whole cell accumulation. In contrast, fabimycin and other amine-containing compounds in Table 1 (including (R)-11) do show significant whole cell accumulation in E. coli.

Given the promising clinical isolate activity, fabimycin was advanced through a battery of mechanistic and translational experiments. While separation of (±)-11 on a preparative chiral column was suitable for the amount of material required for preliminary studies such as MIC assays, it was necessary to optimize the synthetic route to enable the synthesis of quantities of the active enantiomer required for detailed in vivo toxicity, pharmacokinetic, and efficacy experiments. To this end, the synthetic route shown in Scheme 1 was developed and employed to generate gram-scale quantities of enantiopure fabimycin.

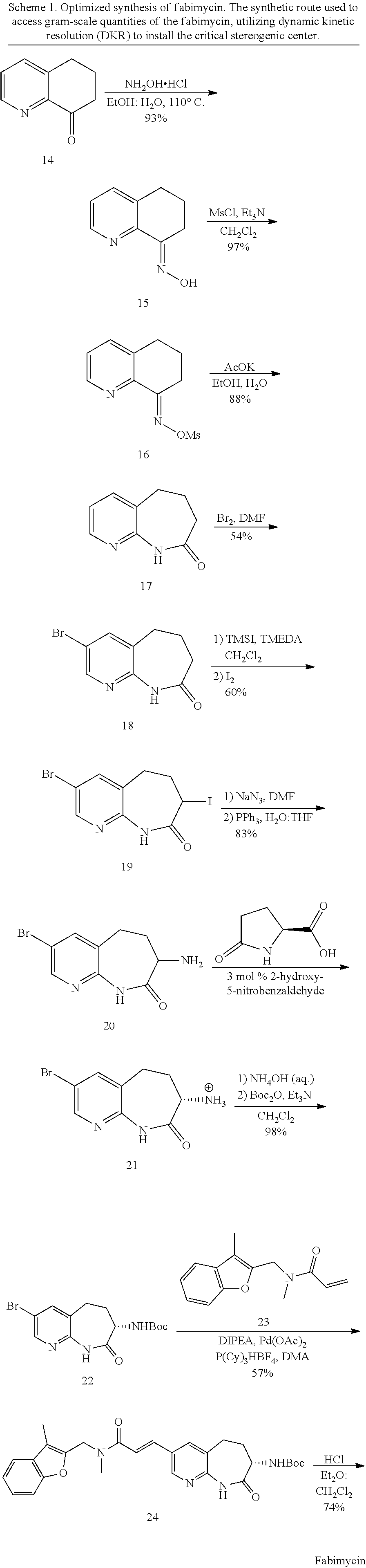

To begin, quinolone 14 was condensed with hydroxylamine, followed by activation of the resulting oxime (15) via treatment with mesyl chloride to generate compound 16 in good yield. After forming azepine 17 using a Beckmann rearrangement, molecular bromine was used to produce aryl bromide 18. Alpha-iodination of 18 provided di-halogenated 19 in good yield, and compound 19 was subjected to azidation and subsequent Staudinger reduction conditions to afford amine 20. This amine was the key intermediate to be enantioenriched via a dynamic kinetic resolution (DKR), proceeding through condensation of the amine onto 2-hydroxy-5-nitrobenzaldehyde and crystallization with L-pyroglutamic acid. Optimization of this critical step involved significant screening. After enantioenrichment, amine 21 was protected, and compound 22 used in a Heck coupling with acrylic amide 23 to produce 24, which, when subjected acidic conditions, liberated fabimycin.

Fabimycin was assessed for its antibacterial activity against a panel of E. coli, K. pneumoniae, A. baumannii, and S. aureus clinical isolates (56 strains in total). As shown in FIG. 1A, fabimycin is markedly more potent than Debio-1452 against all the Gram-negative clinical isolates and has significant potency relative to Debio-1452-NH3 against these strains. All three compounds maintain extremely potent activity versus the panel of S. aureus clinical isolates, with fabimycin being the most potent.

As the greatest improvement in activity of fabimycin (relative to Debio-1452-NH3) was against K. pneumoniae and A. baumannii clinical isolates, fabimycin was further assessed against more diverse and expansive clinical isolate panels of these two pathogens to determine the MIC₅₀ and MIC₉₀ values. Excitingly, when assessed against a panel of 100 K. pneumoniae clinical isolates fabimycin inhibited 90% of the strains at 4 μg/mL, relative to 32 μg/mL for levofloxacin (FIG. 1B). Also encouraging was the narrow MIC range for fabimycin, suggesting that intrinsic resistance to this compound is not prevalent in existing natural bacterial populations. The analogous data in 100 A. baumannii clinical isolates, a panel specifically curated to represent the genomic diversity of the species including even a pan-resistant strain, is also promising, with a MIC₉₀ value of 8 μg/mL (relative to 32 μg/mL for levofloxacin) and a narrow distribution of MIC values (FIG. 1B).

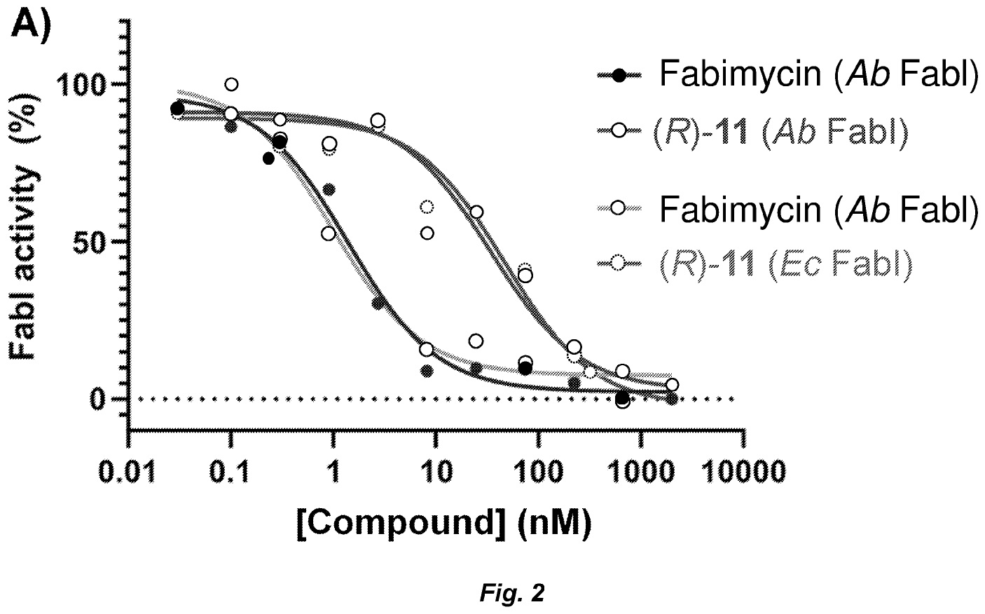

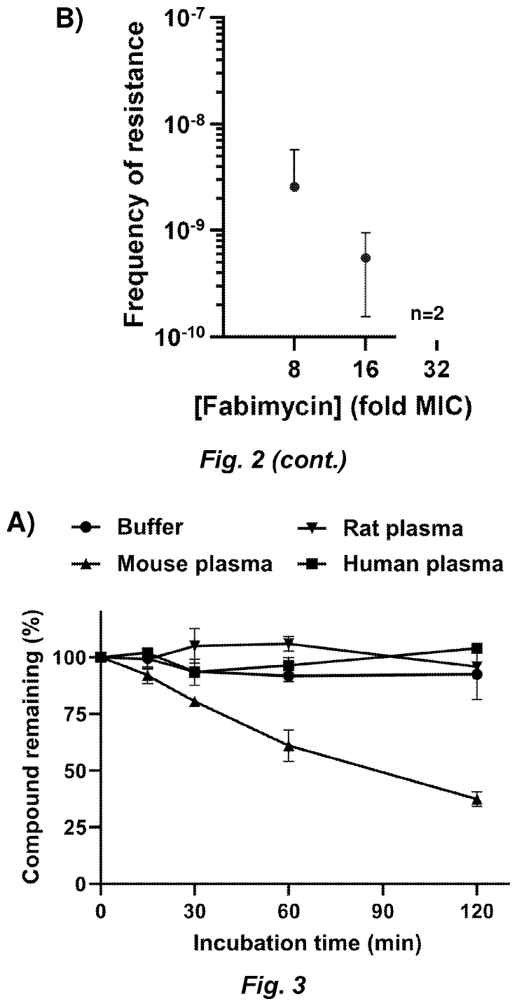

Mode of Action. To probe the effects of varying ring systems and amine placement on FabI target engagement, the apparent inhibition constants (Ki^{app}) for select compounds were determined using purified FabI. The goal of these studies was two-fold, to comparatively assess fabimycin, Debio-1452-NH3, and Debio-1452, and to also assess differential activity against the E. coli and A. baumannii versions of FabI. FabI from E. coli and A. baumannii were recombinantly expressed and purified, and the ability for each compound to inhibit the activity of these enzymes was evaluated using a standard FabI enzyme assay. Fabimycin was the most potent compound against both E. coli and A. baumannii FabI. Of note, expansion from the δ-valerolactam (in Debio-1452-NH3) to the ϵ-caprolactam (in fabimycin) was beneficial as evidenced by a >30-fold change in Ki^{app} (3 vs. 114 nM) (FIG. 2A, Table 2), a fact that seems to be reflected in fabimycin's improved activity against A. baumannii clinical isolates.

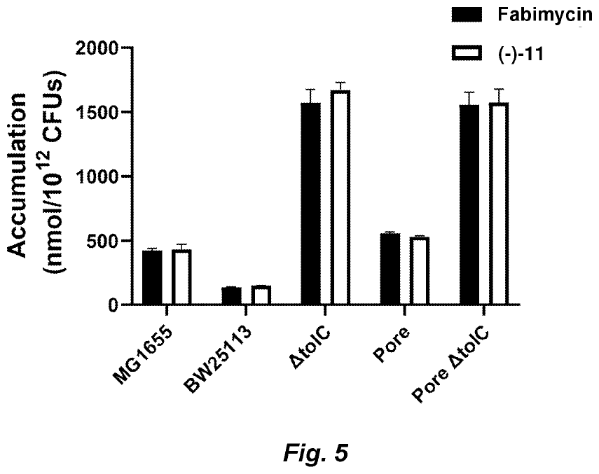

An interesting feature of fabimycin is its considerably enhanced antibacterial activity relative to the opposite enantiomer (R)-11. As mentioned, assessment of accumulation in E. coli revealed that each enantiomer accumulates to a similar extent (FIG. 5). In contrast, evaluation of the two enantiomers in the FabI biochemical enzyme inhibition assay shows a significant activity differential between the two compounds (FIG. 2A, Table 2), with fabimycin being ˜30-fold more potent against A. baumannii FabI. Taken together, the data suggest that the diminished antibacterial activity of (R)-11 relative to fabimycin is due to reduced target engagement and not differential intracellular accumulation.

Spontaneous resistant mutants to fabimycin were generated in E. coli MG1655 at 8×, 16×, and 32× the MIC, with frequencies of resistance ranging 2.06*1⁻¹⁰-9.81*10⁻¹⁰ (FIG. 2B). Importantly, sequencing of the fabI gene revealed mutations leading to single amino acid changes mapping back to the active site of the enzyme (FIG. 2B). Furthermore, while the mutant prevention concentration (MPC) of Debio-1452-NH3 could not be determined due to insolubility at high concentrations in aqueous solution, the MPC of fabimycin was found to be 64 μg/mL using the large-inoculum method with WT E. coli.

TABLE 2
Fabimycin mode of action studies. A) In vitro inhibition of
enzymatic activity of FabI (from A. baumannii and E. coli)
by fabimycin and (R)-11. B) Spontaneous resistance frequency of
E. coli MG1655 to fabimycin, and the resulting mutation
in FabI in the resistant strains. The mutation prevention
concentration (MPC) for fabimycin is 64 μg/mL.
A.

| | A. baumannii FabI | E. coli FabI |
|---|---|---|
| Compound | Ki^{app} (nM) | Ki^{app} (nM) |
| Debio-1452 | 799 ± 229 | 13 ± 1 |
| (±)-Debio-1452-NH3 | 114 ± 22 | 3.5 ± 0.5 |
| (R)-11 | 89 ± 11 | |
| Fabimycin | 3 ± 1 | |

B.

| Fold | FabI | MIC | |
|---|---|---|---|
| MIC | mutant | (μg/mL) | Colonies |
| 8 | Need PCR | | |
| 16 | G148A | 64-128 | 5/13 |
| | G148S | 128 | 5/13 |
| | H209P | 61-128* | 2/13 |
| | WT | 64 | 1/13 |

In-vivo experiments. Given the low resistance frequency and promising data with >200 Gram-negative clinical isolates, experiments were conducted to probe the suitability of fabimycin for in vivo infection models. As a prelude to these studies, fabimycin was evaluated against human cell lines HFF-1 and A549, and these experiments revealed fabimycin to be less cytotoxic relative to Debio-1452-NH3 (Table 3). Next, formulation and maximal tolerated dose (MTD) studies were performed, and in all cases fabimycin was found to be better tolerated in mice with a multi-day single-dose MTD of 200 mg/kg relative to 50 mg/kg for Debio-1452-NH3 over 5 days (Table 4).

TABLE 3
In-vitro cytotoxicity and basic ADME assays.

| | | Debio- | Debio1452- | |
|---|---|---|---|---|
| | | 1452 | NH3 | Fabimycin |
| Mammalian cells | | | | |
| H. sapiens HFF-1 | IC₅₀ (μM) | 165 ± 42 | 63 ± 33 | 112 ± 11 |
| | Inhibition | 18 ± 6 | 11 ± 2 | 9.5 ± 3.4 |
| | at 30 μM (%) | | | |
| H. sapiens A549 | IC₅₀ (μM) | 166 ± 12 | 68 ± 4 | 114 ± 3 |
| | Inhibition | 13 ± 6 | 29 ± 4 | −1.2 ± 3.5 |
| | at 30 μM (%) | | | |
| Additional | | | | |
| ADME studies | | | | |
| HERG IC₅₀ (μM) | | 5.5 | 5.7 | 21.8 |
| Plasma protein | Mouse | 99.4 ± 0.1 | 94.5 ± 0.5 | 96 |
| binding (%) | Human | 98.1 ± 0.2 | 89.3 ± 1.3 | 94 |
| Plasma stability, | Mouse | ND | ND | 82.6 |
| t_{1/2} (min) | Human | ND | ND | 1400 |
| | Rat | ND | ND | |

TABLE 4
Formulation and MTD of amine-containing FabI inhibitors.

In-vivo

| (C57BL/6 mice) | Debio1452-NH3 | Fabimycin | |
|---|---|---|---|
| Formulation | 20% sulfobutyl ether(7) β- | 20% sulfobutyl ether(7) β- | 17% Cremophor EL, 3% |
| | cyclodextrin (SBE-β-CD) | cyclodextrin (SBE-β-CD) | SBE-β-CD in H₂O |
| | in H₂O | in H₂O | |
| Single-dose MTD | 50 mg/kg | >200 mg/kg | 100 mg/kg |
| Multi-day single-dose | 50 mg/kg, 5 d | >100 mg/kg, 5 d | 75 mg/kg, 3 d |
| MTD | | | |
| One-day TID dosing | ND | >200 mg/kg | 75 mg/kg |
| MTD | | | |

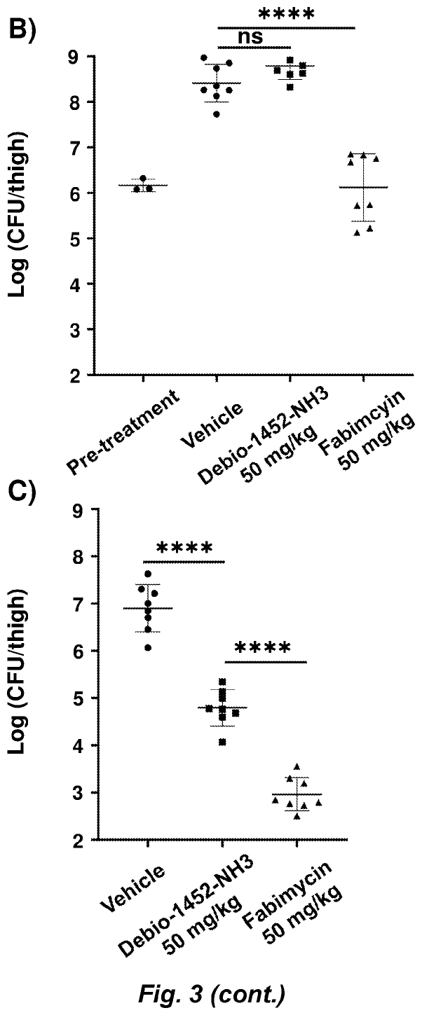

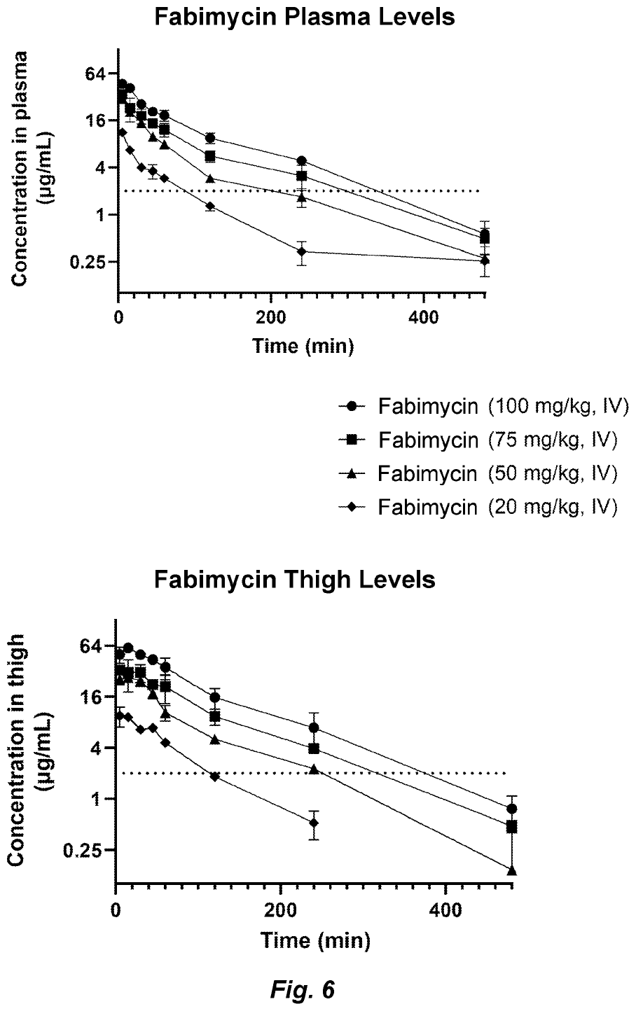

An interesting aspect of this compound class is their serum instability in mice leading to poor pharmacokinetics; for example, while Debio-1452 has a short half-life in mouse serum, it is significantly longer in rat, monkey, and human serum. Assessment of fabimycin in mouse, rat, and human plasma showed a similar trend, with considerable instability in mouse plasma contrasting excellent stability in rat and human plasma (FIG. 3A). While this data suggests the possibility that antibacterial activity could improve fabimycin moves toward humans, it does complicate the evaluation of this compound class in murine infection models. Thus, as a prelude to efficacy studies, a pharmacokinetic study was conducted with fabimycin using neutropenic female BALB/c mice infected with drug-resistant A. baumannii (fabimycin MIC=2 μg/mL) where single doses of the compound (20, 50, 75, 100 mg/kg) were administered to the infected mice and blood taken over the course of 8 hours. Excitingly, when dosed at 100 mg/kg, fabimycin concentrations stayed above the MIC for the infectious strain for over 7 hours in the thigh tissue with the Cₘₐₓ nearing the MPC for WT E. coli (FIG. 6, Table 5).

TABLE 5
Pharmacokinetic analysis of fabimycin.

| | |
|---|---|
| Fabimycin | Fabimycin |
| 100 mg/kg IV | 75 mg/kg IV |

PLASMA

| | | |
|---|---|---|
| AUCₗₐₛₜ (h*μg/mL) | 69.8 | 45.4 |
| t_{1/2} (h) | 1.4 | 1.4 |
| CL (mL/min/kg) | 23.5 | 26.9 |
| Cₘₐₓ (μg/mL) | 47.3 | 34.6 |
| TimeHigh (h) | 6.67 | 5.70 |

THIGH

| | | |
|---|---|---|
| AUCₗₐₛₜ (h*μg/mL) | 112 | 65.2 |
| t_{1/2} (h) | 1.24 | 1.39 |
| CL (mL/min/kg) | 14.7 | 18.9 |
| Cₘₐₓ (μg/g) | 60.2 | 33.0 |
| TimeHigh (h) | 7.19 | 6.22 |

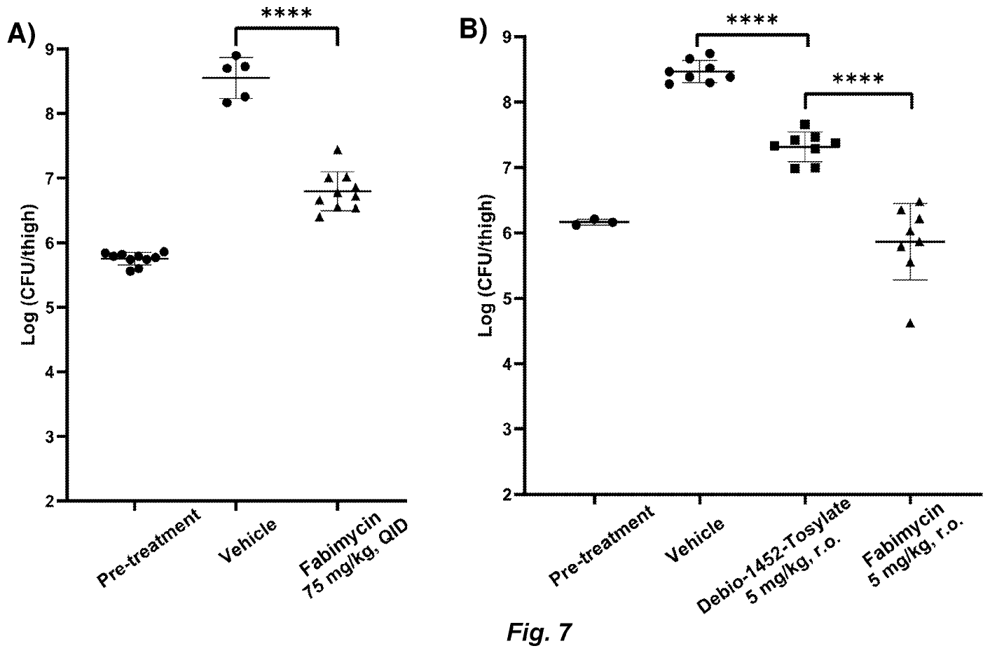

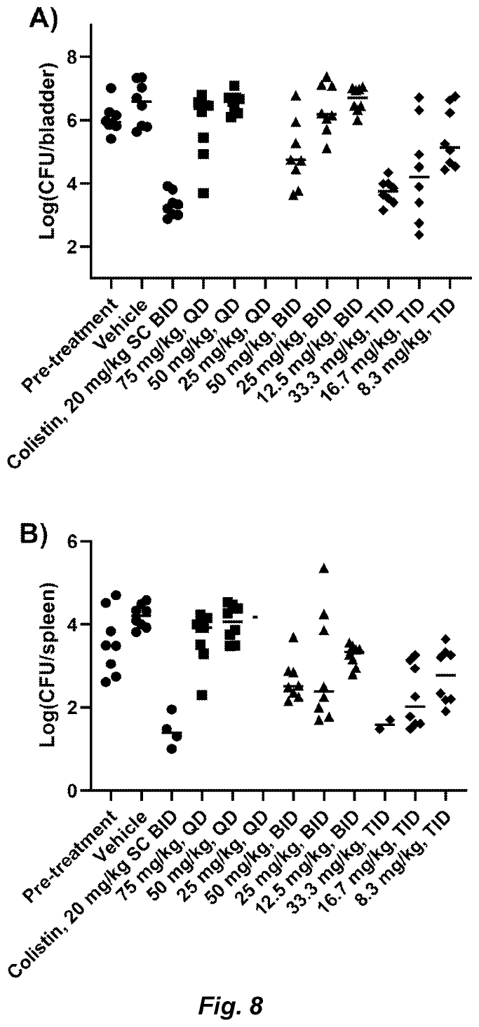

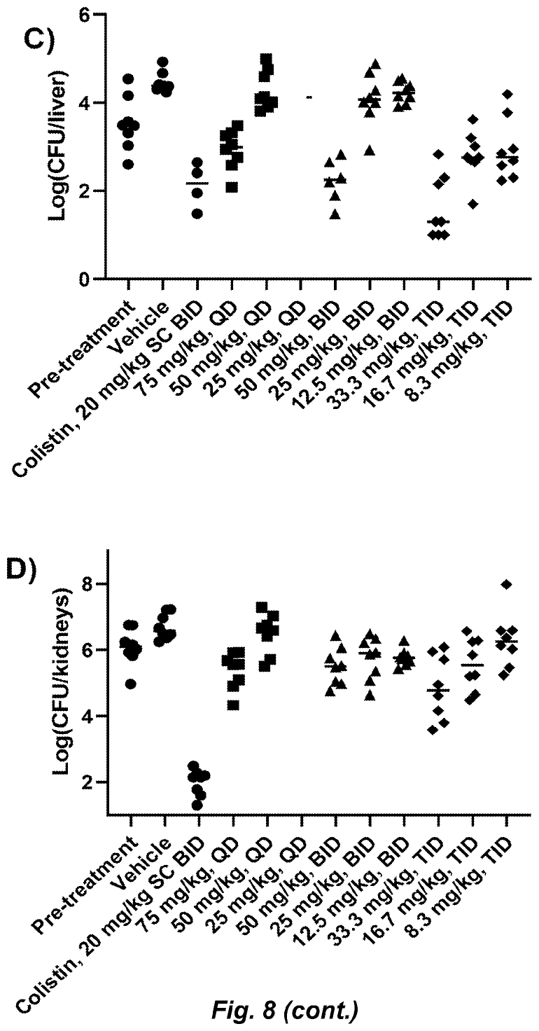

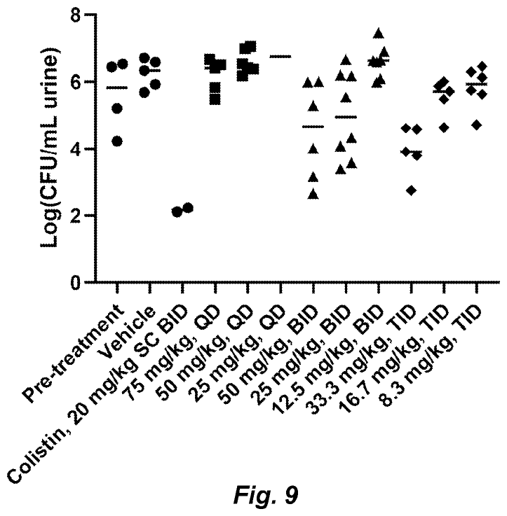

With formulation, MTD, and pharmacokinetic data in hand, the efficacy of fabimycin was evaluated in murine infection models. To start, a comparative assessment was made of fabimycin and Debio-1452-NH3 in two murine neutropenic infection models using a dosing regimen that is at the MTD for Debio-1452-NH3 but below the MTD for fabimycin (three doses, intramuscular, at 50 mg/kg). Using an extensively resistant A. baumannii clinical isolate, fabimycin outperformed Debio-1452-NH3 in both lung and thigh infection models and achieved a >3-fold decrease in log(CFU/lung) and >2-fold decrease log(CFU/thigh) relative to the vehicle (FIG. 3B, 3C). As the goal of these initial models was simply to assess efficacy compared to Debio-1452-NH3, fabimycin dosing was not maximized or optimized. Further evaluation in efficacy in a murine neutropenic thigh infection against extensively-resistant NDM-1 containing strain of A. baumannii found fabimycin able to reduce bacterial burden by nearly 2 log(CFU/thigh) (FIG. 7A). When comparing fabimycin to Debio-1452 in a S. aureus neutropenic thigh infection model fabimycin again showed significantly greater reduction of bacterial burden relative to Debio-1452 when both dosed at 5 mg/kg (FIG. 7B).

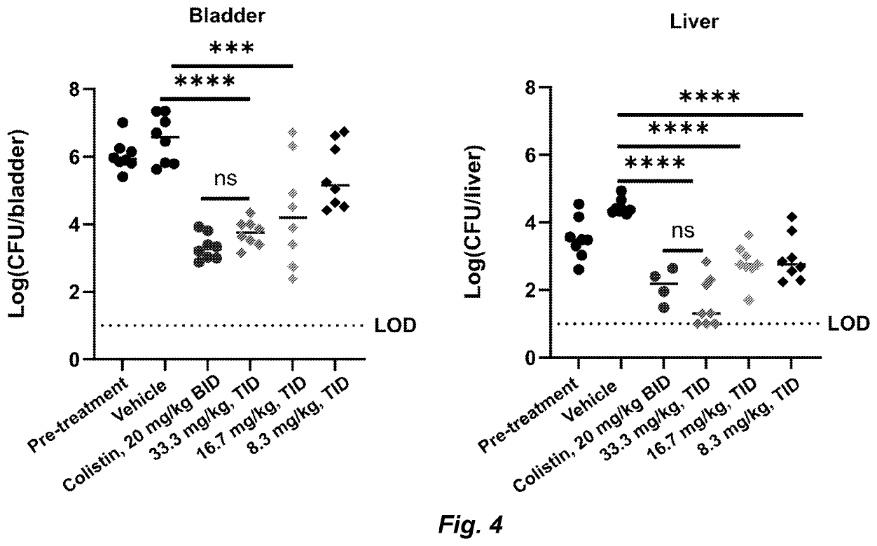

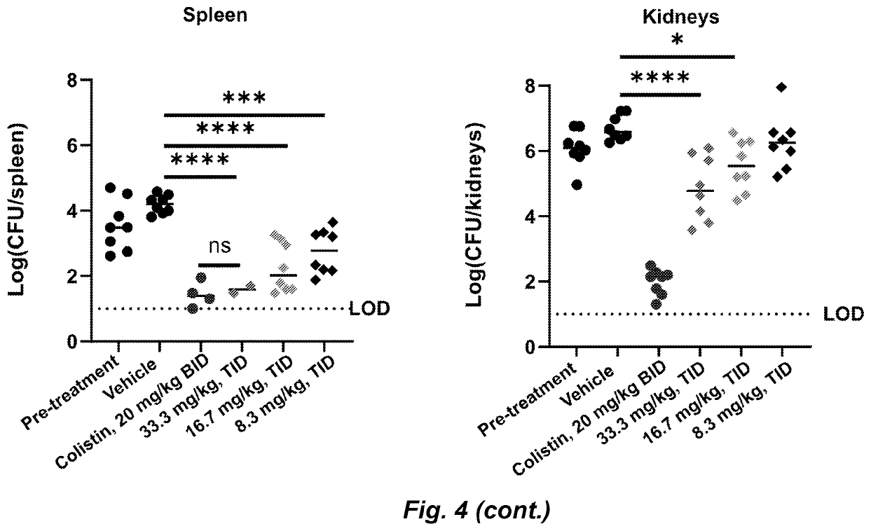

UTIs represent one of the biggest risks for healthy individuals in terms of exposure to antibiotic-resistant bacteria and many individuals contract one in their lifetime (roughly 1 in 2 women and 1 in 10 men); as such, these infections from Gram-negative pathogens, particularly those that are drug-resistant, remain a major clinical challenge. As E. coli is the causative agent in the great majority of UTIs fabimycin was evaluated in a murine UTI model with a challenging, extensively resistant strain of carbapenem resistant E. coli (fabimycin MIC=2 μg/mL). When dosed at 33.3 mg/kg, TID fabimycin was able to achieve 3.0, 2.8, 2.9, and 1.9 log₁₀ reductions in bacterial load relative to the vehicle in the spleen, bladder, liver, and kidneys tissues, respectively (FIG. 4).

TABLE 6
Drug-like properties of fabimycin.

| | | |
|---|---|---|
| Mouse | Rat | Human |

Hepatocyte stability (μL/min/million cells)

| | | |
|---|---|---|
| 6.1 | 0.9 | ND |

Plasma stability, t_{1/2} (min)

| | | |
|---|---|---|
| 83 | ND | 1400 |

Plasma protein binding (%)

| | | |
|---|---|---|
| 96 | ND | 94 |

MDR1-MDCK Permeability (Pₐₚₚ × 10⁻⁶ cm/s)

| A:B | B:A. | Efflux ratio |
|---|---|---|
| 0.6 | 5.7 | 9.4 |

CYP IC₅₀ (μM)

| 1A2 | 2C9 | 2C19 | 2D6 | 3A4 |
|---|---|---|---|---|
| 31 | 27 | 60 | 65 | 7 |

Chart 1. Summary of Fabimycin in vivo models. Log reductions are relative to the vehicle.
S. aureus
   Neutropenic thigh infection w/S. aureus USA300 LAC
      5 mg/kg, r.o.
      26 h, 2.6 log reduction
A. baumannii
   Neutropenic thigh infection w/A. baumannii AR-0299
      50 mg/kg, intramuscular (2, 6, and 11 h post-infection)
      26 h, 2.3 log reduction
   Acute lung infection w/A. baumannii AR-0299
      50 mg/kg, intramuscular (QD)
      48 h, 3.9 log reduction
   Neutropenic thigh infection w/A. baumannii AR-0088
      75 mg/kg IV, QID
      26 h, 1.75 log reduction
E. coli
   Urinary tract infection w/E. coli AR-0055
      33.3 mg/kg IV, TID (100 mg/kg a day)
      168 h post-infection
         Bladder—2.78 log reduction
         Liver—2.85 log reduction
         Spleen—3.04 log reduction
         Kidneys—1.85 log reduction
         Urine—2.32 log reduction

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection, or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-micro-emulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal or vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the invention to the body. Such dosage forms can be made by dissolving or dispersing the active compound in the proper medium. Absorption enhancers can also be used to increase the flux of the compound across the skin. The rate of such flux can be controlled by providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like, into the compositions. In addition, prolonged absorption of the injectable pharmaceutical form may be brought about by inclusion of agents that delay absorption such as aluminum monostearate and gelatin.

In some cases, in order to prolong the effect of a drug, it is desirable to slow the absorption of the drug from subcutaneous or intramuscular injection. This may be accomplished by use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13^{th} ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

A suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The compound can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent, concurrently or sequentially.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid , naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of solvates can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### EXAMPLES

The invention now being generally described, it will be more readily understood by reference to the following examples which are included merely for purposes of illustration of certain aspects and embodiments of the present invention and are not intended to limit the invention.

### Example 1

### Materials and Methods

Bacterial strains. S. aureus ATCC 29213, E. coli MG1655, E. coli BAA-2340, E. coli BAA-2469, E. coli BAA-2471, E. cloacae BAA-2341, E. cloacae BAA-2468, K. pneumoniae BAA-1705, K. pneumoniae BAA-2342, K. pneumoniae BAA-2470, K. pneumoniae BAA-2472, and K. pneumoniae BAA-2473 were obtained from ATCC. E. coli JW5503 and E. coli JW3596 were obtained from the KEIO Collection. E. cloacae ATCC 29893 was provided by W. van der Donk (UIUC). E. coli AR-0346, E. coli AR-0349, E. coli AR-0493, E. coli AR-0495, and K. pneumoniae AR-0347 were obtained from the CDC and FDA AR Isolate Bank. E. coli F20987, E. coli M66623, E. cloacae S28901.1, K. pneumoniae M14723, K. pneumoniae M67198, K. pneumoniae M67297, K. pneumoniae S20595, K. pneumoniae S47889, A. baumannii W41979, A. baumannii F19521, A. baumannii M13100, and A. baumannii WO22 were obtained from the University of Illinois Chicago Medical School. A. baumannii KB304, A. baumannii KB343, and A. baumannii KB357 were provided by J. Quale.

Antimicrobial susceptibility tests. Susceptibility testing was performed in biological triplicate, using the micro-dilution broth method as outlined by the Clinical and Laboratory Standards Institute. Bacteria were cultured with cation-adjusted Muller-Hinton broth (Sigma-Aldrich, Cat# 90922) in round-bottom 96-well plates (Corning, Cat# 3788). Human serum (pooled gender, 0.2 μm filtered) was purchased from BioIVT (Hicksville, NY).

Accumulation assay. The accumulation assay was performed in triplicate in batches of twelve samples [4 compounds total, 3-time points, 1 sample/time point], with each batch containing tetracycline as a positive control. E. coli MG1655 was used for these experiments. For each replicate, 2.5 mL of an overnight culture of E. coli was diluted into 250 mL of fresh Luria Bertani (LB) broth (Lennox) and grown at 37° C. with shaking to an optical density (OD₆₀₀) of 0.70. The bacteria were pelleted at 3,220 r.c.f. for 10 min at 4° C. and the supernatant was discarded. The pellets were re-suspended in 40 ml of phosphate buffered saline (PBS) and pelleted as before, and the supernatant was discarded. The pellets were resuspended in 10.8 mL of fresh PBS and aliquoted into twelve 1.7 mL Eppendorf tubes (890 μL each). The number of colony-forming units (CFUs) was determined by a calibration curve. The samples were equilibrated at 37° C. with shaking for 5 min, compound was added (final concentration=50 μM), and then samples were incubated at 37° C. with shaking for either 10 min, 30 min, 1 hour, or 2 hours. These time points are short enough to minimize metabolic and growth changes (no changes in OD₆₀₀ or CFU count observed). After incubation, 800 μL of the cultures were carefully layered on 700 μL of silicone oil [9:1 AR20 (Acros, Cat#174665000)/Sigma High Temperature (Sigma-Aldrich, Cat#175633), cooled to −78° C.]. Bacteria were pelleted through the oil by centrifuging at 13,000 r.c.f. for 2 min at room temperature (supernatant remains above the oil); the supernatant and oil were then removed by pipetting. To lyse the samples, each pellet was re-suspended in 200 μL of water, and then they were subjected to three freeze-thaw cycle of three minutes in liquid nitrogen followed by three minutes in a water bath at 65° C. The lysates were pelleted at 13,000 r.c.f. for 2 min at room temperature and the supernatant was collected (180 μL). The debris was re-suspended in 100 μL of methanol and pelleted as before. The supernatants were removed and combined with the previous supernatants collected. Finally, remaining debris was removed by centrifuging at 20,000 r.c.f. for 10 min at room temperature. Supernatants were analyzed by LC-MS/MS.

Cell culture. IMR90 cells were obtained from ATCC. IMR90 cells were grown in EMEM with 10% fetal bovine serum (Gemini Benchmark, Cat# 100-106), 100 U/mL penicillin, and 100 μg/mL streptomycin. All cells were cultured at 37° C. in a 5% CO₂ environment. Media was prepared by the Univeristy of Illinois School of Chemical Sciences Cell Media Facility. Sex and age of cell lines: IMR90 (Female, 16 weeks gestation).

Cell viability. Cells seeded (IMR90: 5,000 cells/well) a 96-well plate (Greiner Bio-One, Cat# 655180) and were allowed to attach overnight. Cells were treated with investigational compounds in DMSO (30 μM, 1% DMSO final, 100 μL/well). Raptinal (100 μM) was used as a dead control. After 24 h post-treatment, media was exchanged with compound-free media. After 72 h post-treatment, cell viability was assessed using the Alamar Blue method. Stock Alamar Blue solution [10 μL, 440 μM resazurin (Sigma-Aldrich, Cat# R7017) in sterile 1×PBS] was added to each well, and plate was incubated for 3-4. Conversion of Alamar Blue was measured with plate reader (SpectraMax M3, Molecular Devices) by fluorescence (ex 555 nm, em 585 nm, cutoff 570 nm, autogain). Percent death was determined by normalizing to DMSO-treated cells and raptinal-treated cells.

Molecular docking. Docking of Debio-1452 derivatives into FabI crystal structures was performed with the Small-Molecule Drug Discovery Suite 2018-4 (Schrodinger, New York, NY). Co-crystal structures of Debio-1452 bound to E. coli FabI (PDB: 4JQC) and S. aureus FabI (PDB: 4FS3) were prepared using the Protein Prep Wizard with default settings and used to build receptor grids. For S. aureus FabI allowed rotation for Tyr157 and NADP hydroxyls. For E. coli FabI allowed rotation for Tyr156 and NAD hydroxyls. Positional constraints were applied on the benzofuran ring (center of mass must be within 2 Å of initial position). H-bonding constraints were applied to Tyr156 and Ala95 (E. coli numbering). Ligands were prepared with LigPrep and amines were protonated. Both enantiomers of each ligands were docked with Glide XP. The poses of higher scoring enantiomers were refined and ΔΔG_{bind} was calculated using Prime MM-GBSA. Protein residues within 5 Å of the ligand were sampled using the hierarchical sampling procedure in Prime MM-GBSA.

Selection of resistant mutants. Resistant mutants were selected via the large inoculum method. Briefly, E. coli MG1655 (1.8×10⁹ CFU) were plated on 100 mm plates of LB agar containing 64, 32, and 16 μg/mL Debio-1452-NH₃. Colonies were visible after incubating at 37° C. for 48 h. Resistant colonies were confirmed by streaking on selective media with the same concentration of Debio-1452-NH₃.

Sequencing of fabI. FabI was amplified by colony PCR. Colonies were picked and diluted in 100 μLsterile H₂O. PCR reactions are setup by combining 25 μL MiFi Mix (Bioline, London, UK), 1 μL 20 μM primer mix (EcFabI-PCR-FOR and EcFabI-PCR-REV), 10 μL template, and 14 μL H₂O. Reaction was performed on C1000 Thermal Cycler (Bio-Rad, Hercules, CA) with the following conditions: initial denature 95° C., 3 min; denature 95° C., 15 s; anneal 57° C., 15 s; extend 72° C., 30 s; final extend 3 min; 35 cycles. 5 μL portion of PCR reaction mixture was analyzed by agarose gel to confirm single 1.4 kbp product. PCR reaction was purified using GeneJET PCR Purification Kit (Thermo Scientific). PCR amplicons were submitted to the Core DNA Sequencing Facility at the University of Illinois at Urbana-Champaign for Sanger sequencing with overlapping internal primers (EcFabI-Seq-REV and EcFabI-Seq-REV). All primers were obtained from Integrated DNA Technologies (Coralville, IA).

TABLE 7-1
FabI Sequences.

| Primer | Sequence |
|---|---|
| EcFabI-PCR-FOR | 5′- GGGGCCAGCGTTTCTTTTTC -3′ |
| | (SEQ ID NO: 1) |
| EcFabI-PCR-REV | 5′- AAACATGGAGACGGTGCTGG -3′ |
| | (SEQ ID NO: 2) |
| EcFabI-Seq-FOR | 5′- ATAGCTACTCACAGCCAGGT -3′ |
| | (SEQ ID NO: 3) |
| EcFabI-Seq-REV | 5′- GAAGGGGAGAAAGACGGATC -3′ |
| | (SEQ ID NO: 4) |

Plasmids. Expression vectors for FabI were a generous gift from Peter Tonge (Stonybrook University, NY). Site directed mutagenesis was performed with NEB Q5 Site Directed Mutagenesis Kit according to kit instructions with the primers and annealing temperatures listed below. Mutations were confirmed by Sanger sequencing using T7 promoter and T7 terminator primers.

TABLE 7-2
Species Mutations.

| Species | Mutation | Forward Primer | Reverse Primer | Ta |
|---|---|---|---|---|
| E. coli | A116V | 5′- | 5′- GCCTTCACGGGTAACGGC -3′ | 67 |
| | | TTCAAAATTGTCCACGACATCAG | (SEQ ID NO: 6) | |
| | | CTC -3′ (SEQ ID NO: 5) | | |
| E. coli | G148S | 5′- TTCCTACCTTAGCGCTGAGCG | 5′- AGGGTCAGCAGGGCAGAA -3′ | 67 |
| | | -3′ (SEQ ID NO: 7) | (SEQ ID NO: 8) | |

Expression and purification of E. coli FabI. E. coli BL21 (DE3) pLysS (Novagen) were transformed with pET15-ecFabI. Overnight culture (10 mL) in LB supplemented with 50 μg/mL ampicillin from single colony was diluted into 1 L LB+50 μg/mL ampicillin. Cells were grown at 37° C., 250 rpm until OD₆₀₀ reached 0.8. Culture was cooled to 18° C. and induced with 0.5 mM IPTG for 18 h at 18° C. Cells were harvested by centrifugation (5,000×g, 10 min, 4° C.). Cell pellets were flash frozen and store at −20° C. pending purification. Frozen cell pellets were thawed on ice and resuspended (5 mL per gram wet pellet, typically 20-30 mL) with 0.5% CHAPS, 1 mM PMSF, 1 μg/mL leupeptin, 1 μg/mL pepstatin A, and 2 μg/mL aprotinin in binding buffer [20 mM Tris (pH 7.9), 500 mM NaCl, 5 mM imidazole]. Cells were lysed by sonication on ice (30%, 10 s pulse, 20 s rest, 5 min total). Lysate was clarified by centrifugation (35,000×g, 1 h, 4° C.) and filtration through 0.2 μm syringe filter. Lysate was incubated with 5 mL Co-NTA agarose (HisPur cobalt resin, Thermo-Fisher Scientific Cat#89965, pre-equilibrated with binding buffer) for 30 min at 4° C. with gentle rocking. Agarose-containing lysate was transferred to column and flow through discarded. Column was washed with 2 column volumes of binding buffer followed by 10 column volumes of wash buffer [20 mM Tris (pH 7.9), 500 mM NaCl, 60 mM imidazole]. Protein was eluted with 15 mL elution buffer [20 mM Tris (pH 7.9), 500 mM NaCl, 300 mM imidazole]. Fractions containing protein were identified by SDS-PAGE, subjected to dialysis against FabI storage buffer [60 mM PIPES (pH 8.0), 150 mM NaCl, 1 mM EDTA], and concentrated with Amicon spin filter. Protein solution was aliquoted, flash frozen in liquid nitrogen, and stored at −80° C. Protein concentration was determined by BCA assay (ThermoFisher Cat#23227).

In vitro FabI inhibition assay. NADH (Sigma-Aldrich, Cat# N8129) and crotonoyl-CoA (Sigma-Aldrich, Cat# 28007) were both diluted into activity buffer [100 mM potassium glutamate (pH 7.8)], and working solution was dispensed into two columns of round-bottom 96-well plate (330 μL per well). Inhibitors, dissolved in DMSO at 200× final concentration, were added to each well via multichannel pipette (2.48 μL per well). After thorough mixing, this mixture was transferred to UV-transparent plate (UV-STAR half-area 96-well plate, Greiner Bio-One, Cat#675801, 100 μL per well, three technical replicates per inhibitor concentration). Plate was placed in plate-reader (SpectraMax 190, Molecular Devices) under temperature control (25° C.), and temperature was allowed to equilibrate. Fresh aliquot of enzyme from −80° C. was thawed on ice and diluted in activity buffer. Enzyme working solution was added to plate (50 μL per well, 20 nM enzyme final). After shaking for 5 s, reaction progress was monitored by absorbance at 340 nm every 15 s for 90 min. Linear portion of reaction progress curve was fit using plate-reader software (SoftMax Pro 7.0). Percent activity was calculated relative to DMSO-only and no-enzyme controls in each plate. Percent activity curves were fit to Morrison's quadratic using Graphpad Prism 6.0 to obtain apparent Kᵢ.

Mouse MTD of Debio-1452-NH₃. The protocol was approved by the IACUC at the University of Illinois at Urbana-Champaign (Protocol Number:16144). In these studies, 10- to 12-week-old female C57BL/6 mice purchased from Charles River were used. The maximum tolerate dose (MTD) of single compound was determined first. Debio-1452 amine analogues were formulated in 20% sulfobutyl ether β-cyclodextrin in sterile water. Debio-1452-NH₃ (1), and compounds 2 and 3 were given by IP injection and mice were monitored for signs of toxicity for 2 weeks (single dose). For multiple dose, the compound was given by daily IP for 5 consecutive days and mice were monitored for signs of toxicity for 1 month. MTD was the highest dosage with acceptable toxicity (e.g. <20% weight loss). Single dose MTDs were initially determined. Debio-1452-NH₃ (1) was well tolerated at a single dose of 50 mg kg⁻¹ and compound 3 was well tolerated at a single dose of 100 mg kg⁻¹. Further analysis showed that Debio-1452-NH₃ (1) was well tolerated with daily dosing of 50 mg kg⁻¹ for 5 consecutive days. The MTD of Debio-1452-NH₃ (1) was used to inform the dosing schedule used in subsequent efficacy studies.

Bacterial sepsis survival model. The protocol was approved by the IACUC at the University of Illinois at Urbana-Champaign (Protocol Number: 17271). Six-week-old CD-1 mice were purchased from Charles River and acclimated for 4-7 days. All animals were housed in a pathogen-free environment and received sterile food and water. For the preparation of each inoculum, overnight cultures of clinical isolates were diluted into LB broth and grown to log-phase growth at 37° C. Infection was established via 100 μL retro-orbital injection of bacteria: A. baumannii (W41979) 2.6×10⁸ CFU/mouse, K. pneumoniae (BAA-1705) 1.08×10 8 CFU/mouse, or E. coli (AR-0493) 1.6×10⁸ CFU/mouse. Mice were treated once-a-day for four days with either Debio-1452 Tosylate or Debio-1452-NH₃ (retro-orbital, 50 mg/kg). Drugs were formulated in 20% sulfobutyl ether β-cyclodextrin from solid before treatment. For survival analyses, a Kaplan-Meier Log Rank Survival Test was performed using GraphPad Prism 6.0.

Acute pneumonia bacterial burden model. The protocol was approved by the IACUC at the University of Illinois at Urbana-Champaign (Protocol Number: 17271). Six-week-old CD-1 mice were purchased from Charles River and acclimated for 4-7 days. All animals were housed in a pathogen-free environment and received sterile food and water. For the preparation of each inoculum, overnight cultures of clinical isolates were diluted into LB broth and grown to log-phase growth at 37° C. Lung infection was established via intranasal inoculation of bacteria: A. baumannii (W41979) 2.1×10⁸ CFU/mouse, K. pneumoniae (BAA-1705) 4.4×10⁸ CFU/mouse, or E. coli (AR-0493) 1.73×10⁸ CFU/mouse. Infected mice were then treated once daily for three days with either vehicle, Debio-1452 Tosylate, or Debio-1452-NH₃ (retro-orbital, 50 mg/kg). Drugs were formulated in 20% sulfobutyl ether β-cyclodextrin from solid immediately before treatment. At 48 h post-infection (E. coli) or 72 h post-infection (A. baumannii and K. pneumoniae), CFU were determined in the lungs through serial dilutions. Statistical significance was determined by two-way ANOVA with Tukey's multiple comparison tests.

Statistical analyses. GraphPad Prism 6.0 was used for data analysis and figure generation. Data are shown as the mean±s.e.m. Statistical significance was determined by t-tests (two-tailed) for two groups or two-way ANOVA (with Tukey's multiple comparisons tests) for three or more groups. Survival curves were compared using the log-rank test. P<0.05 was considered statistically significant. In this study, no statistical methods were used to predetermine sample size. The experiments were not randomized, and the investigators were not blinded to allocation during the experiments and outcome assessments.

Code availability. Source code for eNTRyway for local use is available on Github: (github.com/HergenrotherLab/entry-cli).

Materials and Methods for Chemical Synthesis. All reactions were performed under inert atmosphere using nitrogen gas unless otherwise specified. Chemical reagents were purchased from commercial sources and used without further purification. Debio-1452 used for in vitro and cell-based studies was purchased from MedChemExpress. Anhydrous solvents were either purchased from commercial suppliers or dried after being passed through columns packed with activated alumina under positive pressure of nitrogen using a PureSolv MD-5 (Inert previously Innovative Technology inc.) solvent purification system. Final compounds were dried in an Abderhalden drying pistol to remove any residual solvents. ¹H NMR, ¹³C NMR, and 2D NMR experiments for prepared intermediates and products were recorded on a Varian Unity Inova 600 MHz NMR system equipped with an autoX broadband probe and/or a Bruker Avance III HD 500 MHz NMR system equipped with a CryoProbe. Spectra were obtained in the following solvents (reference peaks also included for ¹H and ¹³C NMRs: Deuterated Chloroform-d (¹H NMR 7.26 ppm; ¹³C NMR 77.16 ppm), DMSO-d₆ (¹H NMR 2.50 ppm; ¹³C NMR 39.52 ppm). All the chemical shifts are expressed in ppm (δ), coupling constants (J, Hz) and peak patterns are reported as broad (br), singlet (s), doublet (d), triplet (t), quartet (q) and multiplet (m). High resolution mass spectra (HRMS) were obtained in the School of Chemical Sciences Mass Spectrometry Laboratory on a Waters Q-TOF Ultima quadrupole time of flight spectrometer using electrospray ionization ESI. Purity of the final compounds were purified to ≥95% as assessed by an Agilent Technologies 1290 Infinity II UHPLC equipped with a Phenomenex Kinetex column (2.1 mm ID×50 mm, 1.7 μm particle size, 100 Å pore size).

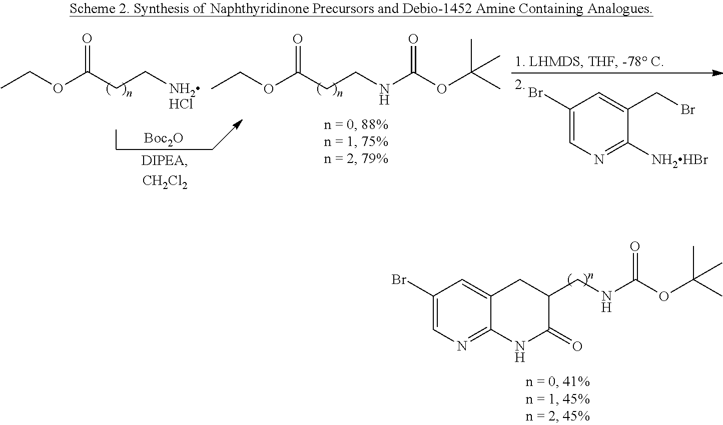

### Synthesis of Debio-1452 Amine Containing Analogues

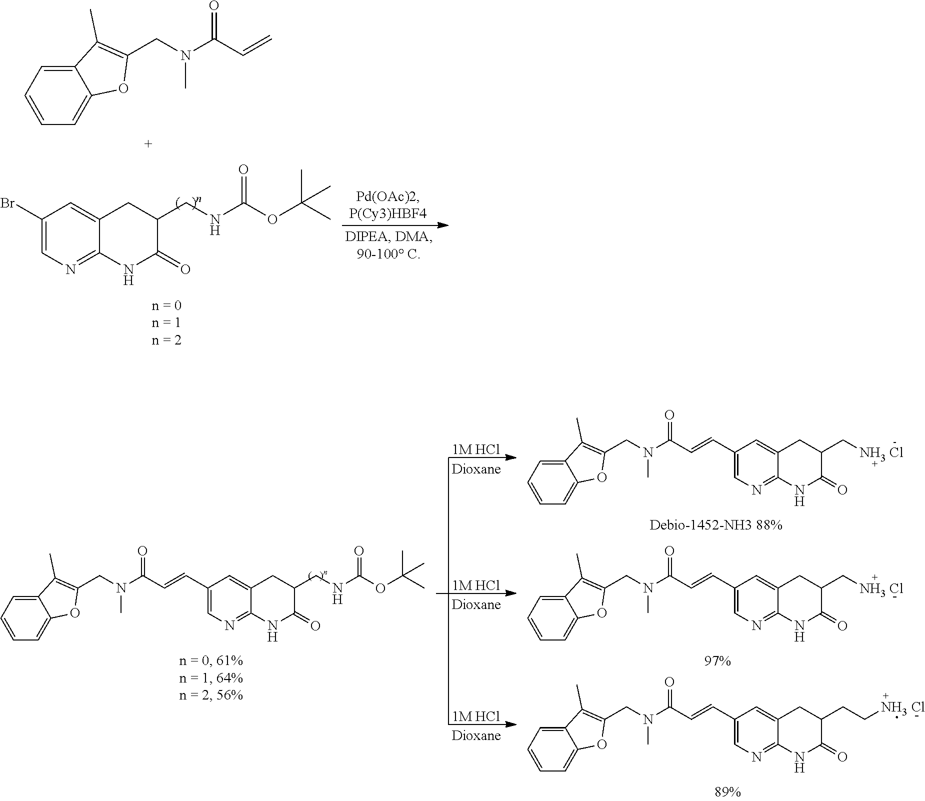

Boc₂O, di-tert-butyl dicarbonate; DIPEA, diisopropylamine; LHMDS, Lithium bis(trimethylsilyl)amide; THF, tetrahydrofuran; Cy, cyclohexyl; DMA, dimethylacetamide.

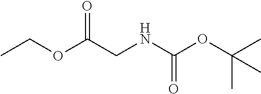

Ethyl (tert-butoxycarbonyl)glycinate—N,N-diisopropylethylamine (2.2 eq, 44 mmol) was added dropwise to a solution of glycine ethyl ester hydrochloride (1 eq, 20 mmol) in CH₂Cl₂ (80 mL) at 0° C. followed by the dropwise addition of di-tert-butyl dicarbonate (1.1 eq, 22 mmol). The reaction mixture was allowed to warm to room temperature and stirred for 1.5 h. The reaction was quenched with saturated aqueous ammonium chloride and extracted with dichloromethane. The combined organic extracts were washed with saturated aqueous sodium bicarbonate, dried over sodium sulfate, and concentrated under reduced pressure. Purification by flash purification column chromatography (10:40:50, EtOAc:CH₂Cl₂:Hexanes) yielded ethyl (tert-butoxycarbonyl)glycinate (3.58 g, 17.6 mmol, 88%) as a colorless oil. ¹H l NMR (500 MHz, Chloroform-d): δ 5.00 (s, 1H), 4.21 (q, J=7.1 Hz, 2H), 3.90 (d, J=5.6 Hz, 2H), 1.45 (s, 9H), 1.28 (t, J=7.2 Hz, 3H). ¹³ C NMR (126 MHz, Chloroform-d): 170.49, 155.83, 80.11, 61.48, 42.62, 28.47, 14.31.

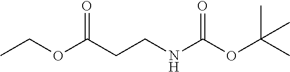

Ethyl 3-((tert-butoxycarbonyl)amino)propanoate—N,N-diisopropylethylamine (2.2 eq, 44 mmol) was added dropwise to a solution of β-alanine ethyl ester hydrochloride (1 eq, 20 mmol) in CH₂Cl₂ (80 mL) at 0° C. followed by the dropwise addition of di-tert-butyl dicarbonate (1.1 eq, 22 mmol). The reaction mixture was allowed to warm to room temperature and stirred for 1.5 h. The reaction was quenched with saturated aqueous ammonium chloride and extracted with dichloromethane. The combined organic extracts were washed with saturated aqueous sodium bicarbonate, dried over sodium sulfate, and concentrated under reduced pressure. Purification by flash purification column chromatography (10:40:50, EtOAc:CH₂Cl₂:Hexanes) yielded ethyl 3-((tert-butoxycarbonyl)amino)propanoate (3.25 g, 15.0 mmol, 75%) as a colorless oil. ¹H NMR (500 MHz, Chloroform-d): δ 5.01 (s, 1H), 4.15 (q, J=7.2 Hz, 2H), 3.48-3.27 (m, 2H), 2.51 (t, J=6.1 Hz, 2H), 1.43 (s, 9H), 1.26 (t, J=7.1 Hz, 3H). ¹³C NMR (126 MHz, Chloroform-d): δ 172.64, 155.92, 79.48, 60.78, 36.26, 34.81, 28.54, 14.35.

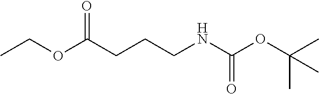 [SMILES: CCOC(=O)CCCNC(=O)OC(C)(C)C]

Ethyl 4-((tert-butoxycarbonyl)amino)butanoate—N,N-diisopropylethylamine (2.2 eq, 44 mmol) was added dropwise to a solution of ethyl 4-aminobutyrate hydrochloride (1 eq, 20 mmol) in CH₂Cl₂ (80 mL) at 0° C. followed by the dropwise addition of di-tert-butyl dicarbonate (1.1 eq, 22 mmol). The reaction mixture was allowed to warm to room temperature and stirred for 1.5 h. The reaction was quenched with saturated aqueous ammonium chloride and extracted with dichloromethane. The combined organic extracts were washed with saturated aqueous sodium bicarbonate, dried over sodium sulfate, and concentrated under reduced pressure. Purification by flash purification column chromatography (10:40:50, EtOAc:CH₂Cl₂:Hexanes) yielded ethyl 4-((tert-butoxycarbonyl)amino)butanoate (3.66 g, 15.8 mmol, 79%) as a colorless oil. ¹H NMR (500 MHz, Chloroform-d): δ 4.62 (s, 1H), 4.13 (q, J=7.2 Hz, 2H), 3.25-3.06 (m, 2H), 2.34 (t, J=7.3 Hz, 2H), 1.81 (p, J=7.2 Hz, 2H), 1.43 (s, 9H), 1.25 (t, J=7.1 Hz, 3H). ¹³C NMR (126 MHz, Chloroform-d): δ 173.42, 156.06, 79.34, 60.59, 40.11, 31.77, 28.55, 25.45, 14.37.

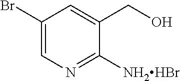

(2-Amino-5-bromopyridin-3-yl)methanol hydrobromide—Bromine (1.01 eq, 39.02 mmol) was added dropwise to a solution of 2-amino-3-(hydroxymethyl)pyridine (1 eq, 38.6 mmol) in glacial acetic acid (60 mL) cooled in an ice bath. After the addition of bromine was complete, the reaction mixture was returned to room temperature. After stirring overnight, the reaction mixture was filtered and washed several times with ether to yield (2-amino-5-bromopyridin-3-yl)methanol hydrobromide (10.01 g, 35.5 mmol, 92% yield) as a yellow solid. (HBr Salt) ¹H NMR (500 MHz, DMSO-d₆): δ 8.17 (d, J=2.3 Hz, 1H), 7.97-7.93 (m, 1H), 4.41 (s, 2H). ¹³ C NMR (126 MHz, DMSO-d₆): δ 151.39, 141.11, 135.60, 127.72, 104.27, 57.98.

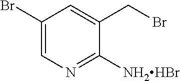

5-Bromo-3-(bromomethyl)pyridin-2-amine hydrobromide—A suspension of (2-amino-5-bromopyridin-3-yl)methanol hydrobromide (1 eq, 35.47 mmol) in 48% hydrobromic acid (70 mL) was refluxed for 10 h. After 10 h, the reaction mixture was allowed to slowly cool to room with stirring, filtered, and rinsed with ethyl acetate. The solid was triturated with ethyl acetate to yield 5-bromo-3-(bromomethyl)pyridin-2-amine hydrobromide (10.226 g, 29.7 mmol, 84%) as a light beige solid. ¹H NMR (500 MHz, DMSO-d₆): δ 8.18 (d, J=2.4 Hz, 1H), 8.15 (d, J=2.4 Hz, 1H), 4.72 (s, 2H). ¹³C NMR (126 MHz, DMSO): δ 153.04, 144.29, 141.01, 121.66, 104.11, 29.13.

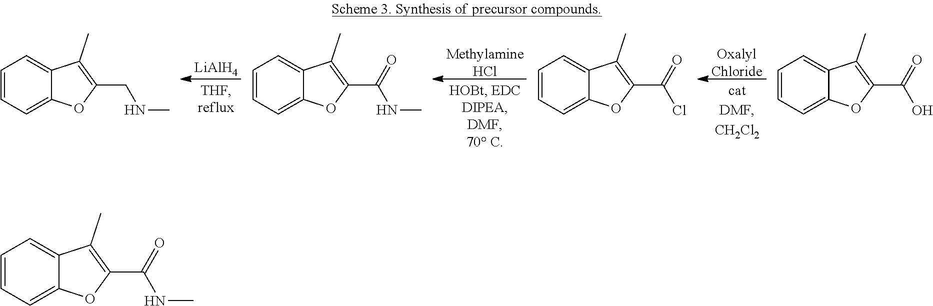

N,3-Dimethylbenzofuran-2-carboxamide—To a solution of 3-methylbenzo[b]furan-2-carboxylic acid (1 eq, 52 mmol), methylamine hydrochloride (1.1 eq, 57.52 mmol), N,N-diisopropylethylamine (2.2 eq, 114.4 mmol), and HOBt (1.1 eq, 57.52 mmol) in DMF (150 mL) was added EDC (1.1 eq, 57.52 mmol). The reaction mixture was heated to 70° C. overnight. The solvent was reduced to a few mL. The crude reaction mixture was diluted with CH₂Cl₂ and washed with saturated aqueous sodium bicarbonate. The organic extracts were dried over sodium sulfate and concentrated under reduced pressure. Purification by flash column chromatography (20:50:30, EtOAc:CH₂Cl_{2 :}hexanes) yielded N,3-dimethylbenzofuran-2-carboxamide (9.24 g, 48.9 mmol, 94%) as a white solid. ¹H l NMR (500 MHz, Chloroform-d): δ7.61 (dt, J=7.8, 1.0 Hz, 1H), 7.45-7.36 (m, 2H), 7.29 (ddd, J=8.0, 6.4, 1.7 Hz, 1H), 6.64 (s, 1H), 3.03 (d, J=5.0 Hz, 3H), 2.63 (s, 3H). ¹³C NMR (126 MHz, Chloroform-d): δ 161.10, 153.35, 142.96, 129.95, 127.04, 123.19, 122.19, 121.07, 111.55, 25.86, 9.00.

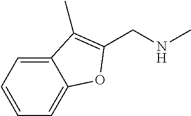

N-Methyl-1-(3-methylbenzofuran-2-yl)methanamine—Lithium aluminum hydride (3 eq, 47.6 mmol) was added portionwise to a solution of N,3-dimethylbenzofuran-2-carboxamide (1 eq, 15.86 mmol) in THF (75 mL) at room temperature. The reaction mixture was refluxed for 11 h. After reaction completion, the reaction mixture was cooled to 0° C. and slowly quenched by the sequential addition of 2 mL water, 2 mL 15% sodium hydroxide, 6 mL water at 15-30 min intervals. The mixture was filtered through a pad of celite rinsed several times with ethyl acetate. Purification by flash column chromatography (5:95, MeOH:CH₂Cl₂) yielded N-methyl-1-(3-methylbenzofuran-2-yl)methanamine (2.513 g, 14.3 mmol, 91%). ¹H NMR (500 MHz, Chloroform-d): δ 7.49-7.44 (m, 1H), 7.43-7.37 (m, 1H), 7.27-7.23 (m, 1H), 7.22 (td, J=7.3, 1.3 Hz, 1H), 3.87 (s, 2H), 2.45 (s, 3H), 2.23 (s, 3H). ¹³C NMR (126 MHz, Chloroform-d): δ 154.25, 151.34, 130.08, 124.01, 122.26, 119.28, 112.33, 111.04, 46.23, 35.80, 8.07.

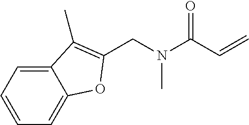 [SMILES: C=CC(=O)N(C)Cc1oc2ccccc2c1C]

N-Methyl-N-((3-methylbenzofuran-2-yl)methyl)aciylamide—N,N-diisopropylethylamine (1.5 eq, 15.4 mmol) was added dropwise to a solution of N-methyl-1-(3-methylbenzofuran-2-yl)methanamine (1 eq, 10.3 mmol) in CH₂Cl₂ (75 mL) at room temperature. After 10 min, acryloyl chloride (2 eq, 20.6 mmol) was added dropwise and the reaction mixture was stirred overnight. The solvent was removed under reduced pressure and purification by flash column chromatography (1:99 to 3:97, MeOH:CH₂Cl₂) yielded N-methyl-N-((3-methylbenzofuran-2-yl)methyl)acrylamide (1.861 g, 8.12 mmol, 79%) as a colorless oil. Note: Tabulated NMR data for acrylamide derivatives consist of two rotamers that exist at room temperature in a ratio of 35:65 and is reflected in the reported integral values. ¹H NMR (500 MHz, Chloroform-d): 7.51-7.45 (m, 1H), 7.43-7.36 (m, 1H), 7.32-7.18 (m, 2H), 6.85 (dd, J=16.8, 10.6 Hz, 0.35H), 6.59 (dd, J=16.7, 10.4 Hz, 0.65H), 6.42-6.33 (m, 1H), 5.80-5.67 (m, 1H), 4.77 (s, 1.3H), 4.62 (s, 0.7H), 3.13 (s, 1.95H), 3.02 (s, 1.05H), 2.29 (s, 1.95H), 2.25 (s, 1.05H). Note: Tabulated NMR data for acrylamide derivatives consist of two rotamers that exist at room temperature which results in doubling of signals for most ¹³C nuclei. ¹³C NMR (126 MHz, Chloroform-d): 167.07, 166.29, 154.26, 154.23, 148.93, 147.52, 129.84, 129.48, 128.48, 128.22, 128.04, 127.54, 124.75, 124.30, 122.59, 122.37, 119.49, 113.74, 113.36, 111.20, 111.07, 45.21, 42.26, 35.36, 33.64, 7.95.

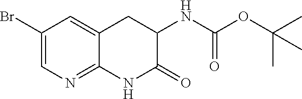 [SMILES: CC(C)(C)OC(=O)NC1Cc2cc(Br)cnc2NC1=O]

Tert-butyl (6-bromo-2-oxo-1,2,3,4-tetrahydro-1,8-naphthyridin-3-yl)carbamate. To a solution of LHMDS (1 M in THF, 4 eq, 33.4 mmol) cooled to −78° C. was added a solution of ethyl (tert-butoxycarbonyl)glycinate (S1, 2 eq, 16.72 mmol) in THF (34 mL) dropwise. The reaction mixture was stirred for 1 h followed by the portion-wise addition (3 portions at 15 min intervals) of 5-bromo-3-(bromomethyl)pyridin-2-amine hydrobromide (1 eq, 8.36 mmol) via a solid addition tube kept under N₂. The reaction mixture was kept at −78° C. for several hours and allowed to warm to −40° C. overnight. The reaction mixture was quenched with 0.5M HCl (aq.) and extracted with ethyl acetate. The combined organic extracts were dried over sodium sulfate and concentrated under reduced pressure. Purification by flash purification column chromatography (01:99 to 10:90, THF:CH₂Cl₂) followed by trituration with ether/n-pentane yielded tert-butyl (6-bromo-2-oxo-1,2,3,4-tetrahydro-1,8-naphthyridin-3-yl)carbamate (1.48 g, 3.46 mmol, 41%) as a white solid. ¹H NMR (500 MHz, Chloroform-d): δ 9.68 (s, 1H), 8.32 (s, 1H), 7.65 (s, 1H), 5.63 (s, 1H), 4.42-4.30 (m, 1H), 3.52 (dd, J=16.4, 6.4 Hz, 1H), 2.83 (t, J=14.9 Hz, 1H), 1.48 (s, 9H). ¹³C NMR (126 MHz, Chloroform-d): δ 169.16, 155.72, 148.68, 147.95, 139.50, 119.83, 114.37, 80.54, 49.85, 31.17, 28.48. HRMS (ESI): m/z calc for C₁₃H₁₆BrN₃O₃ [M+H]⁺: 342.0448, found: 342.0451.

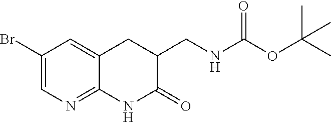 [SMILES: CC(C)(C)OC(=O)NCC1Cc2cc(Br)cnc2NC1=O]

Tert-butyl ((6-bromo-2-oxo-1,2,3,4-tetrahydro-1,8-naphthyridin-3-yl)methyl)carbamate. To a solution of LHMDS (1 M in THF, 4 eq, 36 mmol) cooled to −78° C. was added a solution ethyl 3-((tert-butoxycarbonyl)amino)propanoate (S2, 2 eq, 18 mmol) in THF (36 mL) dropwise. The reaction mixture was stirred for 1.5 h followed by the portionwise addition (3 portions at 15 min intervals) of 5-bromo-3-(bromomethyl)pyridin-2-amine hydrobromide (1 eq, 9 mmol) via a solid addition tube kept under N₂. The reaction mixture was kept at −78° C. for several hours and allowed to warm to −40° C. overnight. The reaction mixture was quenched with 0.5M HCl (aq.) and extracted with ethyl acetate. The combined organic extracts were dried over sodium sulfate and concentrated under reduced pressure. Purification by flash purification column chromatography (01:99 to 10:90, THF:CH₂Cl₂) followed by trituration with ether/n-pentane yielded tert-butyl ((6-bromo-2-oxo-1,2,3,4-tetrahydro-1,8-naphthyridin-3-yl)methyl)carbamate (1.431 g, 4.02 mmol, 45%) as a white solid. ¹H NMR (500 MHz, Chloroform-d): δ 9.38 (s, 1H), 8.28 (s, 1H), 7.63 (s, 1H), 5.31 (d, J=6.9 Hz, 1H), 3.71-3.55 (m, 1H), 3.48 (dt, J=13.7, 6.3 Hz, 1H), 2.95 (dd, J=16.1, 6.9 Hz, 1H), 2.89 (t, J=14.5 Hz, 1H), 2.74 (ddt, J=13.0, 6.7, 3.4 Hz, 1H), 1.43 (s, 9H). ¹³C NMR (126 MHz, Chloroform-d): δ 172.23, 156.45, 149.34, 147.35, 138.99, 120.65, 113.89, 79.69, 40.77, 40.01, 28.53, 27.84. HRMS (ESI): m/z calc for C₁₄H₁₈BrN₃O₃ [M+H]⁺: 356.0604, found: 356.0609.

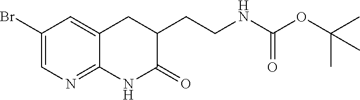 [SMILES: CC(C)(C)OC(=O)NCCC1Cc2cc(Br)cnc2NC1=O]

Tert-butyl (2-(6-bromo-2-oxo-1,2,3,4-tetrahydro-1,8-naphthyridin-3-yl)ethyl)carbamate (7). To a solution of LHMDS (1 M in THF, 4 eq, 18 mmol) cooled to −78° C. was added a solution ethyl 4-((tert-butoxycarbonyl)amino)butanoate (2 eq, 9 mmol) in THF (18 mL) dropwise. The reaction mixture was stirred for 1.5 h followed by the portion-wise addition (2 portions at 15 min intervals) of 5-bromo-3-(bromomethyl)pyridin-2-amine hydrobromide (S4, 1 eq, 4.5 mmol) via a solid addition tube kept under N₂. The reaction mixture was kept at −78° C. for several hours and allowed to warm to −40° C. overnight. The reaction mixture was quenched with 0.5M HCl (aq.) and extracted with ethyl acetate. The combined organic extracts were dried over sodium sulfate and concentrated under reduced pressure. Purification by flash purification column chromatography (01:99 to 10:90, THF:CH₂Cl₂) followed by trituration with ether/n-pentane yielded tert-butyl (2-(6-bromo-2-oxo-1,2,3,4-tetrahydro-1,8-naphthyridin-3-yl)ethyl)carbamate (0.740 g, 2.01 mmol, 45%) as a white solid. ¹H NMR (500 MHz, Chloroform-d): δ 8.73 (s, 1H), 8.25 (d, J=2.2 Hz, 1H), 7.62 (d, J=2.1 Hz, 1H), 4.81 (s, 1H), 3.44-3.29 (m, 1H), 3.28-3.17 (m, 1H), 3.08 (dd, J=16.0, 6.1 Hz, 1H), 2.78 (dd, J=16.0, 9.8 Hz, 1H), 2.65 (dq, J=9.8, 6.6 Hz, 1H), 2.00 (dq, J=13.8, 6.9 Hz, 1H), 1.82-1.67 (m, 1H), 1.43 (s, 9H). ¹³C NMR (126 MHz, Chloroform-d): δ 172.83, 156.19, 149.31, 147.47, 138.98, 120.11, 113.80, 79.53, 38.23, 37.37, 30.33, 29.67, 28.55. HRMS (ES!): m/z calc for C₁₅H₂₀BrN₃O₃ [M+H]⁺: 370.0761, found: 370.0766.

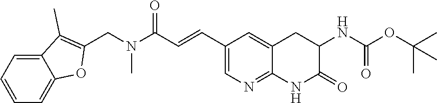 [SMILES: Cc1c(CN(C)C(=O)/C=C/c2cnc3c(c2)CC(NC(=O)OC(C)(C)C)C(=O)N3)oc2ccccc12]

Tert-butyl(E)-(6-(3-(methyl((3-methylbenzofuran-2-yl)methyl)amino)-3-oxoprop-1-en-1-yl)-2-oxo-1,2,3,4-tetrahydro-1,8-naphthyridin-3-yl)carbamate. Anhydrous DMA (10 mL, sparged with N₂ before using) was added to a flask containing N-methyl-N-((3-methylbenzofuran-2-yl)methyl)acrylamide (1.5 eq, 1.875 mmol), tert-butyl (6-bromo-2-oxo-1,2,3,4-tetrahydro-1,8-naphthyridin-3-yl)carbamate (1 eq, 1.25 mmol), palladium(II) acetate (0.2 eq, 0.25 mmol), and tricyclohexylphosphine tetrafluoroborate (0.4 eq, 0.5 mmol) followed by the addition of N,N-diisopropylethylamine (2 eq, 2.5 mmol, distilled and sparged with N₂ before using). The reaction mixture was heated to 90-100° C. for 24 h. After reaction completion, the reaction mixture was diluted with ethyl acetate and filtered through a pad of celite and the filtrate was washed with saturated sodium bicarbonate. The combined organic extracts were dried over sodium sulfate and concentrated under reduced pressure. Purification by flash purification column chromatography (10:90 to 20:00, THF:CH₂Cl₂) followed by trituration with ether/n-pentane yielded tert-butyl (E)-(6-(3-(methyl((3-methylbenzofuran-2-yl)methyl)amino)-3-oxoprop-1-en-1-yl)-2-oxo-1,2,3,4-tetrahydro-1,8-naphthyridin-3-yl)carbamate (0.374 g, 0.762 mmol, 61%) as a white solid. Note: Tabulated NMR data for acrylamide derivatives consist of two rotamers that exist at room temperature in a ratio of 40:60 and is reflected in the reported integral values. ¹H NMR (600 MHz, DMSO-d₆, 25° C.): δ 10.82 (s, 1H), 8.48-8.33 (m, 1H), 8.17-8.02 (m, 1H), 7.60-7.54 (m, 1H), 7.54-7.44 (m, 2.4H), 7.30-7.22 (m, 2H), 7.20 (d, J=15.4 Hz, 0.6H), 7.12-6.97 (m, 1H), 4.98 (s, 0.8H), 4.79 (s, 1.2H), 4.40-4.17 (m, 1H), 3.18 (s, 1.8H), 3.08-2.91 (m, 3.2H), 2.26 (s, 3H), 1.41 (s, 9H). ¹H NMR (600 MHz, DMSO-d₆, 115° C.): δ 10.33 (s, 1H), 8.36 (d, J=2.1 Hz, 1H), 7.97 (s, 1H), 7.55 (d, J=7.5 Hz, 1H), 7.49 (d, J=15.4 Hz, 1H), 7.45 (d, J=8.1 Hz, 1H), 7.28 (t, J=7.6 Hz, 1H), 7.24 (t, J=7.5 Hz, 1H), 7.22-7.16 (m, 1H), 6.57 (d, J=7.8 Hz, 1H), 4.85 (s, 2H), 4.26 (dt, J=13.9, 7.2 Hz, 1H), 3.16-3.06 (m, 4H), 3.05-2.98 (m, 1H), 2.27 (s, 3H), 1.44 (s, 9H). ¹³C NMR (151 MHz, DMSO-d₆, 115° C.): δ 168.63, 165.20, 154.67, 153.13, 150.96, 148.64, 146.37, 137.28, 133.72, 128.96, 125.41, 123.61, 121.76, 118.74, 117.92, 117.39, 112.12, 110.10, 77.93, 49.08, 42.28 (brs, see HSQC) 33.72 (brs), 29.87, 27.66, 6.61. HRMS (ESI): m/z calc for C₂₇H₃₀N₄O₅ [M+H]⁺: 491.2289, found: 491.2302.

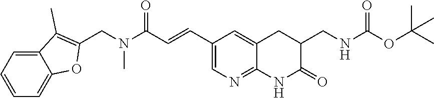 [SMILES: Cc1c(CN(C)C(=O)/C=C/c2cnc3c(c2)CC(CNC(=O)OC(C)(C)C)C(=O)N3)oc2ccccc12]

Tert-butyl(E)-((6-(3-(methyl((3-methylbenzofuran-2-yl)methyl)amino)-3-oxoprop-1-en-1-yl)-2-oxo-1,2,3,4-tetrahydro-1,8-naphthyridin-3-yl)methyl)carbamate. Anhydrous DMA (32 mL, sparged with N₂ before using) was added to a flask N-methyl-N-((3-methylbenzofuran-2-yl)methyl)acrylamide (1.5 eq, 6 mmol), tert-butyl ((6-bromo-2-oxo-1,2,3,4-tetrahydro-1,8-naphthyridin-3-yl)methyl)carbamate (1 eq, 4 mmol), palladium(II) acetate (0.2 eq, 0.8 mmol), and tricyclohexylphosphine tetrafluoroborate (0.4 eq, 1.6 mmol) followed by the addition of N,N-diisopropylethylamine (2 eq, 8 mmol, distilled and sparged with N₂ before using). The reaction mixture was heated to 90-100° C. for 24 h. After reaction completion, the reaction mixture was diluted with ethyl acetate and filtered through a pad of celite and the filtrate was washed with saturated sodium bicarbonate. The combined organic extracts were dried over sodium sulfate and concentrated under reduced pressure. Purification by flash purification column chromatography (10:90 to 20:00, THF:CH₂Cl₂) followed by trituration with ether/n-pentane yielded tert-butyl (E)-((6-(3-(methyl((3-methylbenzofuran-2-yl)methyl)amino)-3-oxoprop-1-en-1-yl)-2-oxo-1,2,3,4-tetrahydro-1,8-naphthyridin-3-yl)methyl)carbamate (1.285 g, 2.55 mmol, 64%) as a white solid. Note: Tabulated NMR data for acrylamide derivatives consist of two rotamers that exist at room temperature in a ratio of 40:60 and is reflected in the reported integral values. ¹H NMR (600 MHz, DMSO-d₆, 25° C.): δ 10.72 (s, 1H), 8.47-8.32 (m, 1H), 8.15-8.02 (m, 1H), 7.59-7.54 (m, 1H), 7.54-7.43 (m, 2.4H), 7.31-7.26 (m, 1H), 7.26-7.17 (m, 1.6H), 6.91-6.81 (m, 1H), 4.99 (s, 0.8H), 4.79 (s, 1.2H), 3.51-3.36 (m, 1H), 3.18 (s, 1.8H), 3.12-2.96 (m, 2H), 2.93 (s, 1.2H), 2.81-2.70 (m, 1H), 2.69-2.59 (m, 1H), 2.26 (s, 3H), 1.45-1.28 (m, 9H). ¹H NMR (600 MHz, DMSO-d₆, 120° C.): δ 10.12 (s, 1H), 8.34 (s, 1H), 7.93 (s, 1H), 7.55 (d, J=7.4 Hz, 1H), 7.49 (d, J=15.5 Hz, 1H), 7.45 (d, J=8.1 Hz, 1H), 7.28 (td, J=8.1, 7.6, 1.5 Hz, 1H), 7.24 (td, J=7.4, 1.1 Hz, 1H), 7.19 (d, J=15.4 Hz, 1H), 6.27 (s, 1H), 4.85 (s, 2H), 3.43 (dt, J=13.6, 5.5 Hz, 1H), 3.18-3.08 (m, 4H), 3.04 (dd, J=15.9, 6.1 Hz, 1H), 2.79 (dd, J=15.8, 10.4 Hz, 1H), 2.75-2.68 (m, 1H), 2.27 (s, 3H), 1.40 (s, 9H). ¹³C NMR (151 MHz, DMSO-d₆, 120° C.): δ 170.73, 165.23, 155.00, 153.12, 151.17, 148.62, 146.07, 137.33, 133.52, 128.94, 125.39, 123.55, 121.71, 118.68, 117.74, 117.70, 112.05, 110.04, 77.38, 43.32 (brs, see HSQC), 39.58, 39.29 (solvent overlap, see HSQC), 33.69 (brs), 27.66, 26.66, 6.54. HRMS (ESI): m/z calc for C₂₈H₃₂N₄O₅ [M+H]⁺: 505.2445, found: 505.2443.

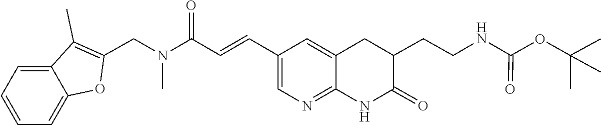 [SMILES: Cc1c(CN(C)C(=O)/C=C/c2cnc3c(c2)CC(CCNC(=O)OC(C)(C)C)C(=O)N3)oc2ccccc12]

Tert-butyl (E)-(2-(6-(3-(methyl((3-methylbenzofuran-2-yl)methyl)amino)-3-oxoprop-1-en-1-yl)-2-oxo-1,2,3,4-tetrahydro-1,8-naphthyridin-3-yl)ethyl)carbamate (10). Anhydrous DMA (10 mL, sparged with N₂ before using) was added to a flask N-methyl-N-((3-methylbenzofuran-2-yl)methyl)acrylamide (1.5 eq, 1.875 mmol), tert-butyl (2-(6-bromo-2-oxo-1,2,3,4-tetrahydro-1,8-naphthyridin-3-yl)ethyl)carbamate (1 eq, 1.25 mmol), palladium(II) acetate (0.2 eq, 0.25 mmol), and tricyclohexylphosphine tetrafluoroborate (0.4 eq, 0.5 mmol) followed by the addition of N,N-diisopropylethylamine (2 eq, 2.5 mmol, distilled and sparged with N₂ before using). The reaction mixture was heated to 90-100° C. for 24 h. After reaction completion, the reaction mixture was diluted with ethyl acetate and filtered through a pad of celite and the filtrate was washed with saturated sodium bicarbonate. The combined organic extracts were dried over sodium sulfate and concentrated under reduced pressure. Purification by flash purification column chromatography (10:90 to 20:80, THF:CH₂Cl₂) followed by trituration with ether/n-pentane yielded tert-butyl (E)-(2-(6-(3-(methyl((3-methylbenzofuran-2-yl)methyl)amino)-3-oxoprop-1-en-1-yl)-2-oxo-1,2,3,4-tetrahydro-1,8-naphthyridin-3-yl)ethyl)carbamate (0.364 g, 0.702 mmol, 56%) as a white solid. Note: Tabulated NMR data for acrylamide derivatives consist of two rotamers that exist at room temperature in a ratio of 40:60 and is reflected in the reported integral values. ¹HNMR (600 MHz, DMSO-d₆, 25° C.): δ 10.70 (s, 1H), 8.52-8.29 (m, 1H), 8.21-8.01 (m, 1H), 7.60-7.55 (m, 1H), 7.54-7.43 (m, 2.4H), 7.33-7.26 (m, 1H), 7.26-7.22 (m, 1H), 7.21 (d, J=16.4 Hz, 0.6H), 6.87 (t, J=5.8 Hz, 1H), 5.00 (s, 0.8H), 4.79 (s, 1.2H), 3.18 (s, 1.8H), 3.12-2.95 (m, 4H), 2.92 (s, 1.2H), 2.78-2.67 (m, 1H), 2.33-2.22 (m, 3H), 1.94-1.84 (m, 1H), 1.47-1.39 (m, 1H), 1.38-1.28 (m, 9H). ¹H NMR (600 MHz, DMSO-d₆, 120° C.): δ 10.05 (s, 1H), 8.34 (s, 1H), 7.93 (s, 1H), 7.55 (d, J=7.6 Hz, 1H), 7.49 (d, J=15.5 Hz, 1H), 7.45 (d, J=8.0 Hz, 1H), 7.32-7.26 (m, 1H), 7.24 (t, J=7.3 Hz, 1H), 7.19 (d, J=15.4 Hz, 1H), 6.29 (s, 1H), 4.85 (s, 2H), 3.14-3.08 (m, 2H), 3.05 (dd, J=15.9, 6.1 Hz, 1H), 2.88 (s, 3H), 2.75 (dd, J=15.9, 10.1 Hz, 1H), 2.63-2.53 (m, 1H), 2.27 (s, 3H), 1.94 (dq, J=13.6, 7.0 Hz, 1H), 1.51 (dq, J=13.9, 6.9 Hz, 1H), 1.39 (s, 9H). ¹³C NMR (151 MHz, DMSO-d₆, 120° C.):): δ 171.96, 165.22, 154.95, 153.11, 151.32, 148.62, 146.10, 137.34, 133.32, 128.94, 125.28, 123.55, 121.71, 118.68, 117.92, 117.68, 112.05, 110.04, 77.01, 42.26, (brs, see HSQC) 37.59, 36.76, 33.66 (brs), 29.37, 28.53, 27.68, 6.54. HRMS (ESI): m/z calc for C₂₉H₃₄N₄O_{5 [}M+H]⁺: 519.2602, found: 519.2616.

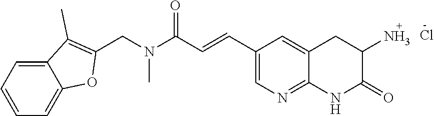 [SMILES: Cc1c(CN(C)C(=O)/C=C/c2cnc3c(c2)CC(N)C(=O)N3)oc2ccccc12 ; Cl]

(E)-3-(6-Amino-7-oxo-5,6,7,8-tetrahydro-1,8-naphthyridin-3-yl)-N-methyl-N-((3-methylbenzofuran-2-yl)methyl)amylamide hydrochloride. Anhydrous 4M HCl in dioxane (1 mL) was added dropwise to a solution of tert-butyl(E)-(6-(3-(methyl((3-methylbenzofuran-2-yl)methyl)amino)-3-oxoprop-1-en-1-yl)-2-oxo-1,2,3,4-tetrahydro-1,8-naphthyridin-3-yl)carbamate (1 eq, 0.652 mmol) in dioxane (3 mL). The reaction mixture was stirred at room temperature. After 4 h, the reaction mixture was concentrated from CH₂Cl₂ several times followed by trituration with ether/n-pentane to afford (E)-3-(6-amino-7-oxo-5,6,7,8-tetrahydro-1,8-naphthyridin-3-yl)-N-methyl-N-((3-methylbenzofuran-2-yl)methyl)acrylamide hydrochloride (244 mg, 0.571 mmol, 88%) as a white solid. Note: Tabulated NMR data for acrylamide derivatives consist of two rotamers that exist at room temperature in a ratio of 40:60 and is reflected in the reported integral values. ¹HNMR (600 MHz, DMSO-d₆, 25° C.): δ 11.33 (s, 1H), 8.79-8.66 (m, 3H), 8.55-8.43 (m, 1H), 8.31-8.20 (m, 1H), 7.62-7.55 (m, 1H), 7.55-7.45 (m, 2.4H), 7.33-7.26 (m, 1.6H), 7.26-7.21 (m, 1H), 5.01 (s, 0.8H), 4.79 (s, 1.2H), 4.44-4.28 (m, 1H), 3.35-3.24 (m, 1H), 3.24-3.06 (m, 2.8H), 2.92 (s, 1.2H), 2.26 (s, 3H). ¹H NMR (600 MHz, DMSO-d₆, 120° C.): δ 10.90 (s, 1H), 8.63 (s, 3H), 8.44 (s, 1H), 8.08 (s, 1H), 7.55 (d, J=7.9 Hz, 1H), 7.51 (d, J=15.4 Hz, 1H), 7.45 (d, J=8.2 Hz, 1H), 7.36-7.18 (m, 3H), 4.86 (s, 2H), 4.28 (dd, J=14.1, 6.9 Hz, 1H), 3.38 (dd, J=15.6, 6.7 Hz, 1H), 3.21 (t, J=14.7 Hz, 1H), 3.11 (s, 3H), 2.27 (d, J=2.3 Hz, 3H). ¹³C NMR (151 MHz, DMSO, 120° C.): δ 166.20, 165.14, 153.13, 150.23, 148.61, 146.69, 136.90, 134.20, 128.95, 126.14, 123.61, 121.76, 118.74, 118.63, 115.77, 112.13, 110.07, 47.37, 42.37 (brs, see HSQC), 33.54 (brs, see HSQC), 27.44, 6.59. HRMS (ESI): m/z calc for C₂₂H₂₂N₄O₃ [M+H]⁺ (Note: hydrochloride salt not 10 observed): 391.1765, found: 391.1773.

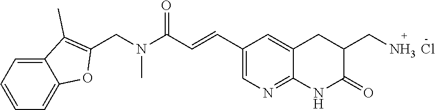 [SMILES: Cc1c(CN(C)C(=O)/C=C/c2cnc3c(c2)CC(CN)C(=O)N3)oc2ccccc12 ; Cl]

(E)-3-(6-(Aminomethyl)-7-oxo-5,6,7,8-tetrahydro-1,8-naphthyridin-3-yl)-N-methyl-N-((3-methylbenzofuran-2-yl)methyl)amylamide hydrochloride (2) -Anhydrous 4M HCl in dioxane (1 mL) was added dropwise to a solution of tert-butyl-(E)-((6-(3-(methyl((3-methylbenzofuran-2-yl)methyl)amino)-3-oxoprop-1-en-1-yl)-2-oxo-1,2,3,4-tetrahydro-1,8-naphthyridin-3-yl)methyl)carbamate (1 eq, 0.6 mmol) in dioxane (3 mL). The reaction mixture was stirred at room temperature. After 4 h, the reaction mixture was concentrated from CH₂Cl₂ several times followed by trituration with ether/n-pentane to afford (E)-3-(6-(aminomethyl)-7-oxo-5,6,7,8-tetrahydro-1,8-naphthyridin-3-yl)-N-methyl-N-((3-methylbenzofuran-2-yl)methyl)acrylamide hydrochloride (256 mg, 0.581 mmol, 97%) as a white solid. Note: Tabulated NMR data for acrylamide derivatives consist of two rotamers that exist at room temperature in a ratio of 40:60 and is reflected in the reported integral values. ¹H NMR (600 MHz, DMSO-d₆, 25° C.): δ 11.03 (s, 1H), 8.49-8.40 (m, 1H), 8.21-8.04 (m, 4H), 7.59-7.55 (m, 1H), 7.55-7.46 (m, 2.4H), 7.31-7.21 (m, 2.6H), 5.01 (s, 0.8H), 4.79 (s, 1.2H), 3.29-3.21 (m, 1H), 3.19 (s, 1.8H), 3.08-2.97 (m, 3H), 2.95-2.85 (m, 2.2H), 2.32-2.19 (m, 3H). ¹H NMR (600 MHz, DMSO-d₆, 120° C.): δ 10.49 (s, 1H), 8.40 (d, J=2.2 Hz, 1H), 8.15 (s, 3H), 7.96 (s, 1H), 7.55 (d, J=7.6 Hz, 1H), 7.50 (d, J=15.5 Hz, 1H), 7.45 (d, J=8.0 Hz, 1H), 7.28 (td, J=8.1, 7.7, 1.5 Hz, 1H), 7.26-7.12 (m, 2H), 4.85 (s, 2H), 3.31-3.27 (m, 1H), 3.14-3.02 (m, 6H), 2.96-2.90 (m, 1H), 2.27 (s, 3H). ¹³C NMR (151 MHz, DMSO-d₆, 120° C.): δ 170.24, 165.20, 153.12, 150.80, 148.63, 146.25, 137.15, 133.57, 128.94, 125.74, 123.59, 121.74, 118.72, 118.20, 117.47, 112.10, 110.06, 42.40 (brs, see HSQC), 38.34, 36.80, 33.73 (brs), 26.66, 6.58. HRMS (ESI): m/z calc for C₂₃H₂₄N₄O₃ [M+H]⁺ (Note: hydrochloride salt not observed): 405.1921, found: 405.1927.

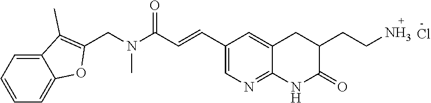 [SMILES: Cc1c(CN(C)C(=O)/C=C/c2cnc3c(c2)CC(CCN)C(=O)N3)oc2ccccc12 ; Cl]

(E)-3-(6-(Aminoethyl)-7-oxo-5,6,7,8-tetrahydro-1,8-naphthyridin-3-yl)-N-methyl-N-((3-methylbenzofuran-2-yl)methyl)amylamide hydrochloride. Anhydrous 4M HCl in dioxane (1 mL) was added dropwise to a solution of tert-butyl-(E)-(2-(6-(3-(methyl((3-methylbenzofuran-2-yl)methyl)amino)-3-oxoprop-1-en-1-yl)-2-oxo-1,2,3,4-tetrahydro-1,8-naphthyridin-3-yl)ethyl)carbamate (1 eq, 0.6 mmol) in dioxane (3 mL). The reaction mixture was stirred at room temperature. After 4 h, the reaction mixture was concentrated from CH₂Cl₂ several times followed by trituration with ether/pentane to afford (E)-3-(6-(aminoethyl)-7-oxo-5,6,7,8-tetrahydro-1,8-naphthyridin-3-yl)-N-methyl-N-((3-methylbenzofuran-2-yl)methyl)acrylamide hydrochloride (244 mg, 0.536 mmol, 89%) as a white solid. Note: Tabulated NMR data for acrylamide derivatives consist of two rotamers that exist at room temperature in a ratio of 40:60 and is reflected in the reported integral values. ¹H NMR (600 MHz, DMSO-d₆, 25° C.): δ 10.80 (s, 1H), 8.47 — 8.37 (m, 1H), 8.17 — 8.09 (m, 1H), 8.06 — 7.95 (m, 3H), 7.60 — 7.54 (m, 1H), 7.54 — 7.43 (m, 2.4H), 7.30 — 7.26 (m, 1H), 7.26 — 7.17 (m, 1.6H), 4.99 (s, 0.8H), 4.79 (s, 1.2H), 3.18 (s, 1.8H), 3.03 — 2.97 (m, 1H), 2.96 — 2.89 (m, 3.2H), 2.80 — 2.67 (m, 2H), 2.30 — 2.22 (m, 3H), 2.10 — 2.00 (m, 1H), 1.71 — 1.63 (m, 1H). ¹H NMR (600 MHz, DMSO-d₆, 115° C.): δ 10.27 (s, 1H), 8.38 (s, 1H), 7.96 (s, 1H), 7.83 (s, 3H), 7.55 (d, J=7.6 Hz, 1H), 7.50 (d, J=15.4 Hz, 1H), 7.45 (d, J=8.1 Hz, 1H), 7.28 (t, J=7.7 Hz, 1H), 7.24 (t, J=7.4 Hz, 1H), 7.20 (d, J=15.4 Hz, 1H), 4.85 (s, 2H), 3.11 (s, 3H), 3.05 (dd, J=15.4, 5.7 Hz, 1H), 3.03 — 2.89 (m, 2H), 2.79 (dd, J=15.4, 11.3 Hz, 1H), 2.76-2.69 (m, 1H), 2.27 (s, 3H), 2.10 (dq, J=14.4, 7.4 Hz, 1H), 1.77 (dq, J=13.8, 7.0 Hz, 1H). ¹³C NMR (151 MHz, DMSO-d₆, 115° C.): δ 171.63, 165.22, 153.13, 151.19, 148.64, 146.21, 137.35, 133.50, 128.96, 125.46, 123.63, 121.78, 118.76, 117.92, 117.85, 112.13, 110.09, 42.25 (brs, see HSQC), 36.76, 36.57, 33.71 (brs), 28.73, 27.07, 6.62. HRMS (ESI): m/z calc for C₂₄H₂₆N₄O₃ [M+H]⁺ (Note: hydrochloride salt not observed): 419.2078, found: 419.2071.

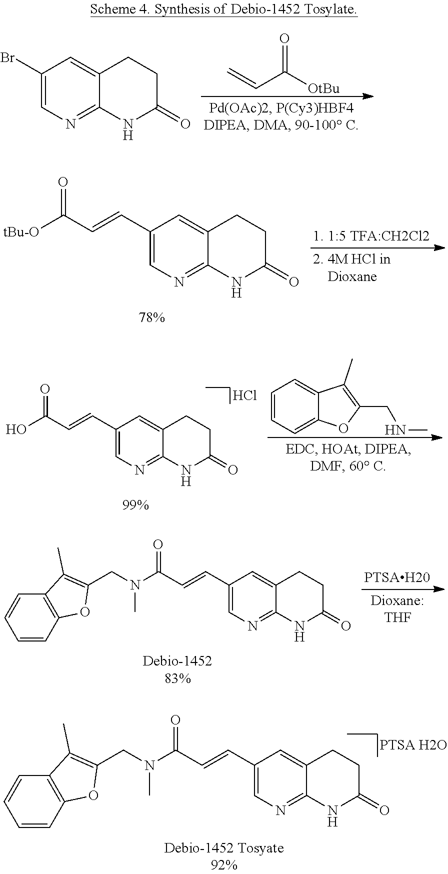

Cy, cyclohexyl; DIPEA, N,N-diisopropyl ethylamine ; TFA, trifluoroacetic acid; EDC, 1-ethyl-3-(3-dimethylaminopropyl)carbodiimide; HOAt, 1-hydroxy-7-azabenzotriazole; DMF, N,N-Dimethylformamide; PTSA, p-toluene sulfonic acid; THF, tetrahydrofuran.

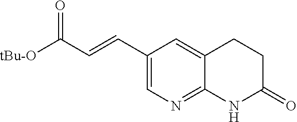

Tert-butyl (E)-3-(7-oxo-5,6,7,8-tetrahydro-1,8-naphthyridin-3-yl)amylate. Anhydrous DMA (32 mL, sparged with N₂ before using) was added to a flask containing 6-bromo-3,4-dihydro-1,8-naphthyridin-2(M)-one (1 eq, 10 mmol), palladium(II) acetate (0.05 eq, 0.5 mmol), and tricyclohexylphosphine tetrafluoroborate (0.1 eq, 1.0 mmol) followed by the addition of tert-butyl acrylate (1.5 eq, 15 mmol, sparged with N₂ before using), N,N-diisopropylethylamine (2 eq, 20 mmol, distilled and sparged with N₂ before using). The reaction mixture was heated to 90-100° C. for 24 h. After reaction completion, the reaction mixture was diluted with ethyl acetate and filtered through a pad of celite and the filtrate was washed with saturated sodium bicarbonate. The combined organic extracts were dried over sodium sulfate and concentrated under reduced pressure. Purification by flash purification column chromatography (10:90 to 30:70, EtOAc:CH₂Cl₂) followed by trituration with ether/n-pentane yielded tert-butyl (E)-3-(7-oxo-5,6,7,8-tetrahydro-1,8-naphthyridin-3-yl)acrylate (2.151 g, 7.85 mmol, 78%) as a white solid. ¹H NMR (500 MHz, Chloroform-d): δ 8.94 (s, 1H), 8.32 (d, J=2.1 Hz, 1H), 7.65 (d, J=1.5 Hz, 1H), 7.51 (d, J=16.0 Hz, 1H), 6.33 (d, J=16.0 Hz, 1H), 2.99 (t, J=7.6 Hz, 2H), 2.71 (dd, J=8.4, 6.8 Hz, 2H), 1.53 (s, 9H). ¹³C NMR (126 MHz, Chloroform-d): 170.97, 165.97, 151.95, 147.37, 139.43, 134.05, 126.16, 120.57, 118.84, 80.99, 77.36, 30.40, 28.34, 24.22.

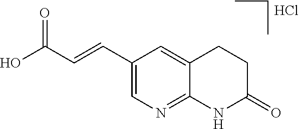

(E)-3-(7-Oxo-5,6,7,8-tetrahydro-1,8-naphthyridin-3-yl)amylic acid hydrochloride. Tert-butyl-(E)-3-(7-oxo-5,6,7 ,8-tetrahydro-1,8-naphthyridin-3-yl)acrylate was dissolved trifluoroacetic acid:CH₂Cl₂ (8 mL:40 mL) and stirred at room temperature. After 2 h, the reaction mixture was concentrated several times from CH₂Cl₂. The crude material was suspended in 4 M HCl in dioxane (20 mL), stirred for 30 min, filtered, and rinsed with ether to afford (E)-3-(7-oxo-5,6,7,8-tetrahydro-1,8-naphthyridin-3-yl)acrylic acid HCl (7.67 mmol, 99%) as a white solid. ¹H NMR (500 MHz, DMSO-d₆): δ 10.68 (s, 1H), 8.35 (d, J=2.2 Hz, 1H), 8.02 (d, J=2.1 Hz, 1H), 7.54 (d, J=16.0 Hz, 1H), 6.51 (d, J=16.0 Hz, 1H), 2.91 (t, J=7.6 Hz, 2H), 2.53 (dd, J=8.5, 6.8 Hz, 2H). ¹³C NMR (126 MHz, DMSO): δ 171.01, 167.47, 152.77, 147.33, 140.62, 133.78, 124.72, 119.20, 118.34, 29.97, 23.27.

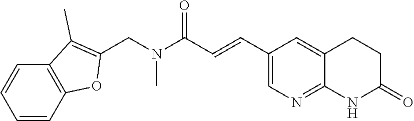 [SMILES: Cc1c(CN(C)C(=O)/C=C/c2cnc3c(c2)CCC(=O)N3)oc2ccccc12]

(E)-N-Methyl-N-((3-methylbenzofuran-2-yl)methyl)-3-(7-oxo-5,6,7,8-tetrahydro-1,8-naphthyridin-3-yl)amylamide-To a solution of N-methyl-1-(3-methylbenzofuran-2-yl)methanamine (1.1 eq, 6.93 mmol), (E)-3-(7-oxo-5,6,7,8-tetrahydro-1,8-naphthyridin-3-yl)acrylic acid hydrochloride (1 eq, 6.3 mmol), 1-hydroxy-7-azabenzotriazole (1.1 eq, 6.93 mmol), in N,N-Dimethylformamide (32 mL) was added N,N-diisopropylethylamine (2.2 eq, 13.86 mmol) followed by 1-ethyl-3-(3-dimethylaminopropyl)carbodiimide (1.1 eq, 6.93 mmol). The reaction mixture was heated to 60° C. for 6 h. The crude reaction mixture was diluted with water, filtered, rinsed with water, rinsed with ether, and dried to afford (E)-N-methyl-N-((3-methylbenzofuran-2-yl)methyl)-3-(7-oxo-5,6,7,8-tetrahydro-1,8-naphthyridin-3-yl)acrylamide (1.970 g, 5.25 mmol, 83%) as a light beige solid. Note: Tabulated NMR data for acrylamide derivatives consist of two rotamers that exist at room temperature in a ratio of 40:60 and is reflected in the reported integral value. ¹H NMR (500 MHz, DMSO-d₆): δ 10.65 (s, 1H), 8.41-8.34 (m, 1H), 8.18-8.00 (m, 1H), 7.59-7.54 (m, 1H), 7.54-7.45 (m, 2.4H), 7.31-7.26 (m, 1H), 7.26-7.22 (m, 1H), 7.22-7.16 (m, 0.6H), 4.99 (s, 0.8H), 4.79 (s, 1.2H), 3.18 (s, 1.8H), 2.92 (s, 1.2H), 2.92-2.87 (m, 2H), 2.57-2.51 (m, 2H), 2.26 (s, 3H).

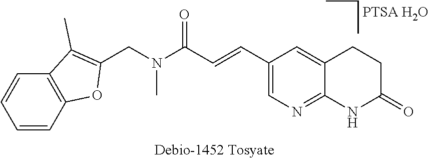

(E)-N-Methyl-N-((3-methylbenzofuran-2-yl)methyl)-3-(7-oxo-5,6,7,8-tetrahydro-1,8-naphthyridin-3-yl)amylamide p-toluenesulfonic acid monohydride—Debio-1452 (1 eq, 1.5 mmol) was suspended in THF (120 mL) and heated to reflux. After 30 min, p-toluene sulfonic acid monohydrate (1.05 eq, 1.58 mmol) in dioxane (12 mL) was added to the reaction mixture and stirred for 1 h. The reaction mixture was allowed to cool to room temperature and diluted with a mixture of 1:1 ether:n-pentane (80 mL), filtered, rinsed with 1:1 ether:n-pentane, and dried to afford (E)-N-methyl-N-((3-methylbenzofuran-2-yl)methyl)-3-(7-oxo-5,6,7,8-tetrahydro-1,8-naphthyridin-3-yl)acrylamide p-toluenesulfonic acid (Debio-1452 Tosylate, 0.756 g, 1.38 mmol, 92%) as a white solid. The product was further processed for in vivo efficacy studies to improve solubility. For these studies, Debio-1452 Tosylate was ground in a mortar and pestle and then sieved through a 75 μM mesh. Note: Tabulated NMR data for acrylamide derivatives consist of two rotamers that exist at room temperature in a ratio of 40:60 and is reflected in the reported integral values. ¹H NMR (500 MHz, DMSO-d₆): δ 10.69 (s, 1H), 9.49 (brs, 1H), 8.42-8.32 (m, 1H), 8.17-8.05 (m, 1H), 7.59-7.54 (m, 1H), 7.54-7.43 (m, 4.4H), 7.31-7.22 (m, 2H), 7.21 (d, J=12.6 Hz, 0.6H), 7.15-7.09 (m, 2H), 4.99 (s, 0.8H), 4.79 (s, 1.2H), 3.18 (s, 1.8H), 2.96-2.89 (m, 3.2H), 2.57-2.51 (m, 2H), 2.29 (s, 3H), 2.26 (s, 3H).

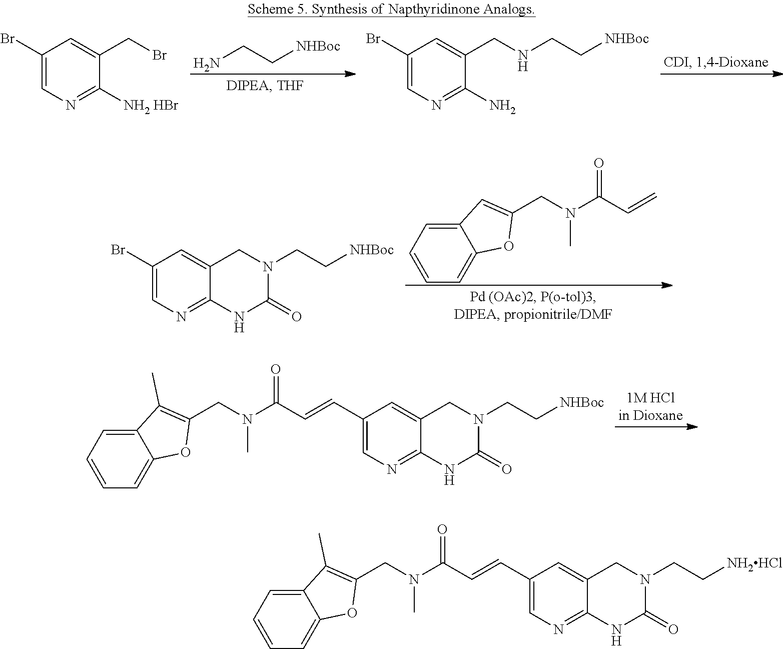

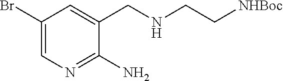 [SMILES: CC(C)(C)OC(=O)NCCNCc1cc(Br)cnc1N]

Tert-butyl (2-(((2-amino-5-bromopyridin-3-yl)methyl)amino)ethyl)carbamate. DIPEA (2, 1.74 mmol) was added to a solution of 5-bromo-3-(bromomethyl)pyridin-2-amine hydrobromide (1 eq, 0.87 mmol) in THF (8 mL) cooled to 0° C. followed by the immediate dropwise addition of tert-butyl(aminoethyl)carbamate (3 eq, 2.61 mmol). The reaction mixture was allowed to stir to room temperature and stirred for 2 h. After reaction completion, water was added to the reaction mixture and the product was extracted with diethyl ether. The combined organic extracts were dried over sodium sulfate and concentrated under reduced pressure. Purification by flash purification column chromatography (00:100 to 05:95, MeOH:CH₂Cl₂) yielded tert-butyl (2-(((2-amino-5-bromopyridin-3-yl)methyl)amino)ethyl)carbamate (0.276 g, 0.80 mmol, 92%). ¹H NMR (500 MHz, Chloroform-d) δ 8.02 (d, T=2.4 Hz, 1H), 7.37 (d, T=2.3 Hz, 1H), 5.50 (s, 2H), 4.65 (s, 1H), 3.73 (s, 2H), 3.25 (q, T=5.9 Hz, 2H), 2.70 (t, J=5.9 Hz, 2H), 1.44 (s, 9H).

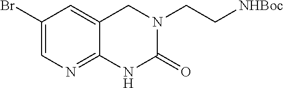 [SMILES: CC(C)(C)OC(=O)NCCN1Cc2cc(Br)cnc2NC1=O]

Tert-butyl (2-(6-bromo-2-oxo-1,4-dihydropyrido[2,3-d]pyrimidin-3(2H)-yl)ethyl)carbamate. Carbonyldiimidazole (1.2 eq, 0.96 mmol) was added to a solution of tert-butyl (2-(((2-amino-5-bromopyridin-3-yl)methyl)amino)ethyl)carbamate (1 eq, 0.80 mmol) in 1,4-dioxane (5 mL) and heated to reflux for 12 h. The reaction mixture was cooled to room temperature and concentrated. Purification by flash purification column chromatography (20:80 THF:CH₂Cl₂) followed by trituration with hexanes yielded tert-butyl (2-(6-bromo-2-oxo-1,4-dihydropyrido[2,3-d]pyrimidin-3(2H)-yl)ethyl)carbamate (0.193 g, 0.52 mmol, 65%). ¹H NMR (500 MHz, Chloroform-d) δ 8.20 (d, T=2.3 Hz, 1H), 7.68 (s, 1H), 7.48 (s, 1H), 4.92 (s, 1H), 4.52 (s, 2H), 3.56 (t, J=6.2 Hz, 2H), 3.38 (q, J=6.1 Hz, 2H), 1.39 (s, 9H).

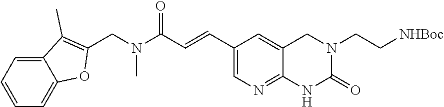 [SMILES: Cc1c(CN(C)C(=O)/C=C/c2cnc3c(c2)CN(CCNC(=O)OC(C)(C)C)C(=O)N3)oc2ccccc12]

Tert-butyl (E)-(2-(6-(3-(methyb(3-methylbenzofuran-2-yl)methyl)amino)-3-oxoprop-1-en-1-yl)-2-oxo-1,4-dihydropyrido[2,3-d]pyrimidin-3(2H)-yl)ethyl)carbamate. Propionitrile (4 mL, sparged with N₂ before using) was added to a flask containing N-methyl-N-((3-methylbenzofuran-2-yl)methyl)acrylamide (1.1 eq, 0.297 mmol), tert-butyl (2-(6-bromo-2-oxo-1,4-dihydropyrido[2,3-d]pyrimidin-3(2H)-yl)ethyl)carbamate (1 eq, 0.27 mmol), palladium(II) acetate (0.2 eq, 0.054 mmol), tri(o-tolyl)phosphine (0.4 eq, 0.108 mmol), and DIPEA (4 eq, 1.08 mmol). The reaction mixture was heated to 90-100° C. for 1 day. After reaction completion, the reaction mixture was diluted with dichloromethane and filtered through a pad of celite and the filtrate was washed with water. The combined organic extracts were dried over sodium sulfate and concentrated under reduced pressure. Purification by flash purification column chromatography (10:90 to 20:80, THF:CH₂Cl₂) followed by trituration with ether/n-heptane yielded tert-butyl (E)-(2-(6-(3-(methyl((3-methylbenzofuran-2-yl)methyl)amino)-3-oxoprop-1-en-1-yl)-2-oxo-1,4-dihydropyrido[2,3-d]pyrimidin-3(2H)-yl)ethyl)carbamate (0.040 g, 0.078 mmol, 29%) as a white solid. ¹H NMR (500 MHz, DMSO-d₆) δ 9.53 (s, 1H), 8.33 (d, J=2.1 Hz, 1H), 7.90 (d, J=2.0 Hz, 1H), 7.55 (d, J=7.9 Hz, 1H), 7.53 (s, 2H), 7.30-7.22 (m, 2H), 6.55 (s, 1H), 4.85 (s, 2H), 4.51 (s, 2H), 3.40 (t, J=6.2 Hz, 2H), 3.18 (q, J=6.2 Hz, 3H), 3.06 (s, 3H), 2.26 (s, 3H), 1.35 (s, 9H).

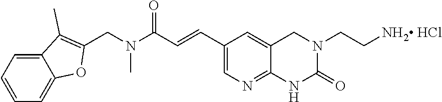 [SMILES: Cc1c(CN(C)C(=O)/C=C/c2cnc3c(c2)CN(CCN)C(=O)N3)oc2ccccc12 ; "· HCl"]

(E)-3-(3-(2-Aminoethyl)-2-oxo-1,2,3,4-tetrahydropyrido[2,3-d]pyrimidin-6-yl)-N-methyl-N-((3-methylbenzofuran-2-yl)methyl)aciylamide hydrochloride. Anhydrous 4M HCl in dioxane (0.5 mL) was added dropwise to a solution of tert-butyl (E)-(2-(6-(3-(methyl((3-methylbenzofuran-2-yl)methyl)amino)-3-oxoprop-1-en-1-yl)-2-oxo-1,4-dihydropyrido[2,3-d]pyrimidin-3(2H)-yl)ethyl)carbamate (1 eq, 0.058 mmol) in dioxane (1.5 mL). The reaction mixture was stirred at room temperature. After 4 h, the reaction mixture was concentrated from CH₂Cl₂ several times followed by trituration with ether/pentane to afford ((E)-3-(3-(2-aminoethyl)-2-oxo-1,2,3,4-tetrahydropyrido[2,3-d]pyrimidin-6-yl)-N-methyl-N-((3-methylbenzofuran-2-yl)methyl)acrylamide hydrochloride (23 mg, 0.05 mmol, 87%) as a white solid. ¹H NMR (500 MHz, DMSO-d6) 6 10.06 (s, 1H), 8.45-8.33 (m, 1H), 8.01-7.94 (m, 1H), 7.88 (s, 3H), 7.59-7.54 (m, 1H), 7.54-7.41 (m, 2.4H), 7.32-7.21 (m, 2H), 7.17 (d, J=15.3 Hz, 0.6H), 4.98 (s, 0.8H), 4.79 (s, 1.2H), 4.53 (s, 2H), 3.66-3.50 (m, 2H), 3.18 (s, 1.8H), 3.10-2.97 (m, 2H), 2.93 (s, 1.2H), 2.26 (s, 3H).

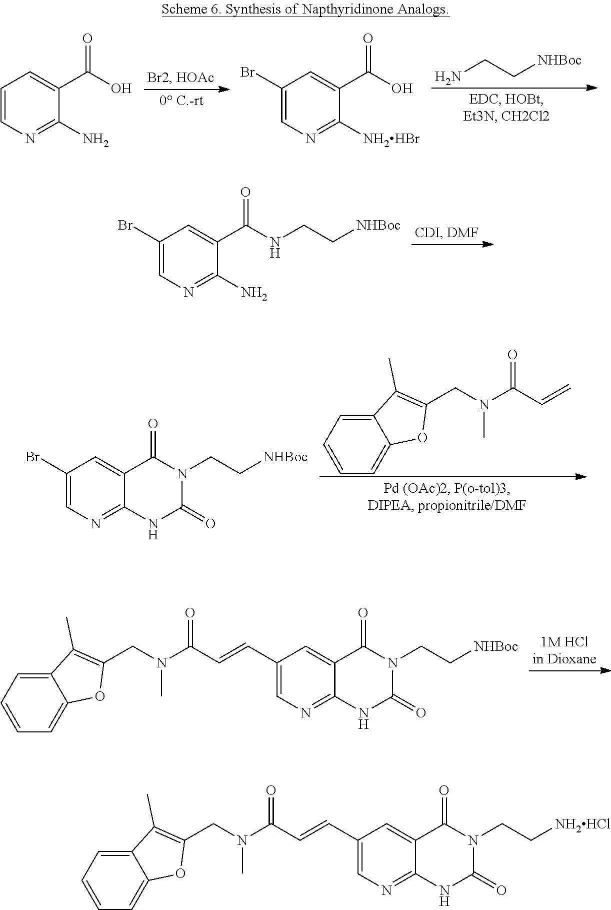

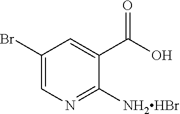

2-Amino-5-bromo-nicotinic acid hydrobromide. Bromine (1.01 eq, 36.49 mmol) was added dropwise to a suspension of 2-amino-nicotinic acid (1 eq, 36.13 mmol) in glacial acetic acid (55 mL) cooled in an ice bath. After the addition of bromine was complete, the mixture was stirred at room temperature overnight. The solid was filtered and rinsed with ether to afford 2-amino-5-bromo-nicotinic acid hydrobromide (9.77 g, 33.02, 91%) as a yellow solid. ¹H NMR (500 MHz, DMSO-d₆) δ 8.33 (d, J=2.6 Hz, 1H), 8.21 (d, J=2.5 Hz, 1H). ¹³C NMR (126 MHz, DMSO) δ 166.82, 156.91, 150.85, 143.19, 108.72, 103.73.

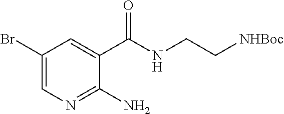 [SMILES: CC(C)(C)OC(=O)NCCNC(=O)c1cc(Br)cnc1N]

Tert-butyl (2-(2-amino-5-bromonicotinamido)ethyl)carbamate. To a suspension of 2-amino-5-bromo-nicotinic acid hydrobromide (1 eq, 3.38 mmol) in CH₂Cl₂ (35 mL) was added triethylamine (1.5 eq, 5.07 mmol), EDC (1.05 eq, 3.55 mmol), and HOBt (1.05 eq, 3.55 mmol) at 0° C. The mixture was stirred for 10 min followed by the addition of N-Boc-ethylenediamine (1.05 eq, 3.55 mmol). The reaction mixture was allowed to warm to room temperature and stirred overnight. After reaction completion, the mixture was extracted with CH₂Cl₂ from 2M NaOH (aq). The combined organic extracts were dried over sodium sulfate and concentrated. Purification by flash purification column chromatography (05:95 MeOH:CH₂Cl₂) followed by trituration with diethyl ether afforded tert-butyl (2-(2-amino-5-bromonicotinamido)ethyl)carbamate (0.844 g, mmol, 70%) as a white solid. ¹H NMR (500 MHz, CDCl₃) δ 8.16 (d, J=2.3 Hz, 1H), 7.82 (d, J=2.3 Hz, 1H), 7.51 (s, 1H), 6.44 (s, 2H), 4.99 (s, 1H), 3.48 (m, 2H), 3.41 (m, 2H), 1.46 (s, 9H).

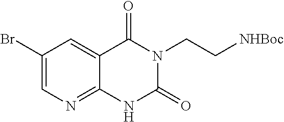 [SMILES: CC(C)(C)OC(=O)NCCn1c(=O)[nH]c2ncc(Br)cc2c1=O]

Tert-butyl (2-(6-bromo-2-oxo-1,4-dihydropyrido[2,3-d]pyrimidin-3(2H)-yl)ethyl)carbamate. Carbonyldiimidazole (1.2 eq, 0.67 mmol) was added to a solution of tert-butyl (2-(((2-amino-5-bromopyridin-3-yl)methyl)amino)ethyl)carbamate (1 eq, 0.56 mmol) in DMF (5 mL) and heated to 80° C. for 12 h. The reaction mixture was cooled to room temperature and concentrated. Purification by flash purification column chromatography (10:90 to 20:80 THF:CH₂Cl₂) followed by trituration with ethyl acetate/diethyl ether yielded tert-butyl (2-(6-bromo-2,4-dioxo-1,4-dihydropyrido[2,3-d]pyrimidin-3(2H)-yl)ethyl)carbamate 0.118 g, 0.31 mmol, 55%) as a white solid. ¹H NMR (500 MHz, DMSO-d₆) δ 12.09 (s, 1H), 8.73 (d, J=2.5 Hz, 1H), 8.39 (d, J=2.4 Hz, 1H), 6.84 (t, J=6.2 Hz, 1H), 3.94 (t, J=5.7 Hz, 2H), 3.19 (q, J=6.0 Hz, 2H), 1.28 (s, 9H).

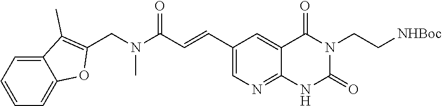 [SMILES: Cc1c(CN(C)C(=O)/C=C/c2cnc3[nH]c(=O)n(CCNC(=O)OC(C)(C)C)c(=O)c3c2)oc2ccccc12]

Tert-butyl (E)-(2-(6-(3-(methyl((3-methylbenzofuran-2-yl)methyl)amino)-3-oxoprop-1-en-1-yl)-2,4-dioxo-1,4-dihydropyrido[2,3-d]pyrimidin-3(2H)-yl)ethyl)carbamate. Propionitrile (4 mL, sparged with N₂ before using) and DMF (2 mL, sparged with N₂ before using) was added to a flask containing N-methyl-N-((3-methylbenzofuran-2-yl)methyl)acrylamide (1.1 eq, 0.29 mmol), yielded tert-butyl (2-(6-bromo-2,4-dioxo-1,4-dihydropyrido[2,3-d]pyrimidin-3(2H)-yl)ethyl)carbamate (1 eq, 0.26 mmol), palladium(II) acetate (0.2 eq, 0.052 mmol), tri(o-tolyl)phosphine (0.4 eq, 0.104 mmol), and DIPEA (4 eq, 1.04 mmol). The reaction mixture was heated to 90-100° C. for 1 day. After reaction completion, the reaction mixture was diluted with dichloromethane and filtered through a pad of celite and the filtrate was washed with water. The combined organic extracts were dried over sodium sulfate and concentrated under reduced pressure. Purification by flash purification column chromatography (10:90 to 20:80, THF:CH₂Cl₂) followed by trituration with ether/n-heptane yielded tert-butyl (E)-(2-(6-(3-(methyl((3-methylbenzofuran-2-yl)methyl)amino)-3-oxoprop-1-en-1-yl)-2,4-dioxo-1,4-dihydropyrido [2,3-d]pyrimidin-3(2H)-yl)ethyl)carbamate (0.051 g, 0.0955 mmol, 37%) as a white solid. ¹H NMR (500 MHz, Chloroform-d) δ 8.92 (s, 1H), 8.77-8.66 (m, 1H), 8.66-8.56 (m, 1H), 7.80-7.67 (m, 1H), 7.57-7.46 (m, 1H), 7.46-7.38 (m, 1H), 7.36-7.19 (m, 2H), 7.02 (d, J=15.4 Hz, 1H), 4.85 (s, 2H), 4.74 (s, 1H), 4.35-4.12 (m, 2H), 3.61-3.37 (m, 2H), 3.27 (s, 2H), 3.11 (s, 1H), 2.33 (s, 3H), 1.31 (s, 9H).

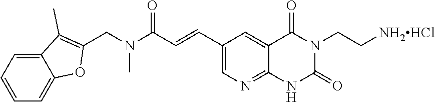

(E)-3-(3-(2-Aminoethyl)-2,4-dioxo-1,2,3,4-tetrahydropyrido[2,3-d]pyrimidin-6-yl)-N-methyl-N-((3-methylbenzofuran-2-yl)methyl)acrylamide hydrochloride. Anhydrous 4M HCl in dioxane (0.5 mL) was added dropwise to a solution of tert-butyl (E)-(2-(6-(3-(methyl((3-methylbenzofuran-2-yl)methyl)amino)-3-oxoprop-1-en-1-yl)-2-oxo-1,4-dihydropyrido [2,3-d] pyrimidin-3(2H)-yl)ethyl)carbamate (1 eq, 0.075 mmol) in dioxane (1.5 mL). The reaction mixture was stirred at room temperature. After 4 h, the reaction mixture was concentrated from CH₂Cl₂ several times followed by trituration with ether to afford (E)-3-(3-(2-aminoethyl)-2,4-dioxo-1,2,3,4-tetrahydropyrido[2,3-d]pyrimidin-6-yl)-N-methyl-N-((3-methylbenzofuran-2-yl)methyl)acrylamide hydrochloride (27 mg, 0.057 mmol, 76%) as a white solid. ¹H NMR (500 MHz, DMSO-d₆) δ 12.33-12.16 (m, 1H), 9.02-8.97 (m, 1H), 8.78 (d, J=2.3 Hz, 0.4H), 8.71 (d, J=2.3 Hz, 0.6H), 7.83 (s, 3H), 7.74-7.62 (m, 1.4H), 7.60-7.54 (m, 1H), 7.52-7.41 (m, 1.6H), 7.33-7.21 (m, 2H), 5.05 (s, 0.8H), 4.80 (s, 1.2H), 4.26-4.06 (m, 2H), 3.18 (s, 1.8H), 3.13-3.03 (m, 2H), 2.95 (s, 1.2H), 2.32-2.25 (m, 3H).

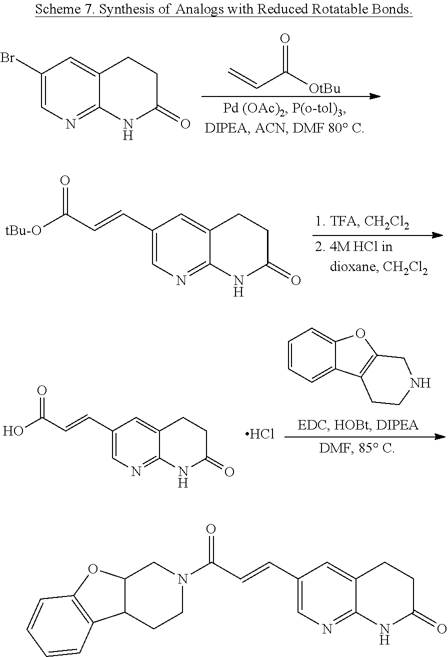

Other benzofuroazepine analogs can be prepared using the procedures described herein.

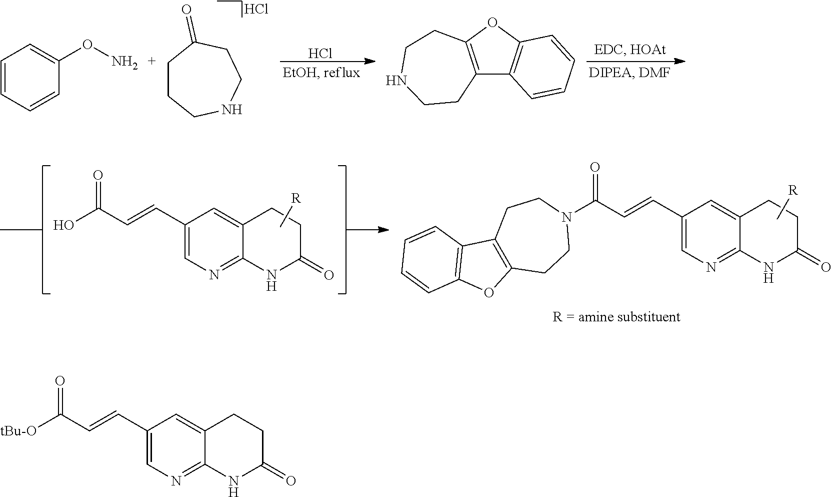

Tert-butyl (E)-3-(7-oxo-5,6,7,8-tetrahydro-1,8-naphthyridin-3-yl)aciylate. A solution of 6-bromo-3,4-dihydro-1H-[1,8]napthyridin-2-one (1 eq, 5.285 mmol), tert-butyl acrylate (1.5 eq, 7.93 mmol), and DIPEA (1.5 eq, 7.93 mmol) in acetonitrile (30 mL) and DMF (8 mL) were degassed with N₂ for 20 min followed by the addition of palladium(II) acetate (0.05 eq, 0.264 mmol), tri(o-tolyl)phosphine (0.1 eq, 0.529 mmol). The reaction mixture was heated to 80° C. for 12 h. Upon reaction completion, the reaction mixture was concentrated. The residue was taken up in MeOH/CH₂Cl₂, filtered through a pad of celite and the filtrate was washed with water. The combined organic extracts were dried over sodium sulfate and concentrated under reduced pressure. Purification by flash purification column chromatography (05:95 to 10:90, THF:CH₂Cl₂) followed by trituration with cold ethanol yielded tert-butyl (E)-3-(7-oxo-5,6,7,8-tetrahydro-1,8-naphthyridin-3-yl)aciylate (0.640 g, 2.33 mmol, 44%). ¹H NMR (500 MHz, Chloroform-d) δ 8.94 (s, 1H), 8.32 (d, J=2.1 Hz, 1H), 7.65 (s, 1H), 7.51 (d, J=16.0 Hz, 1H), 6.33 (d, J=16.0 Hz, 1H), 2.99 (t, J=7.6 Hz, 2H), 2.71 (dd, J=8.4, 6.8 Hz, 2H), 1.53 (s, 9H). ¹³C NMR (126 MHz, CDCl₃) δ 170.97, 165.97, 151.95, 147.37, 139.43, 134.05, 126.16, 120.57, 118.84, 80.99, 30.40, 28.34, 24.22.

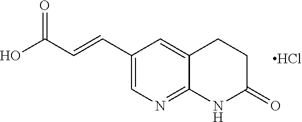 [SMILES: O=C(O)/C=C/c1cnc2c(c1)CCC(=O)N2 ; Cl]

(E)-3-(7-Oxo-5,6,7,8-tetrahydro-1,8-naphthyridin-3-yl)aciylic acid hydrochloride. To a suspension of yielded tert-butyl (E)-3-(7-oxo-5,6,7,8-tetrahydro-1,8-naphthyridin-3-yl)aciylate (1 eq, 2.18 mmol) in CH₂Cl₂ (10 mL) was added trifluoroacetic acid (2 mL). The reaction mixture was stirred for 6 h at room temperature. After reaction completion, the reaction mixture was concentrated several times from CH₂Cl₂ under reduced pressure. The crude product was suspended in 4M HCl in dioxane (10 mL), sonicated, filtered, and rinsed with diethyl ether to afford—(0.555 g, 2.18 mmol, quantitative) as a white solid. ¹H NMR (500 MHz, DMSO-d₆) δ 10.70 (s, 1H), 8.35 (d, J=2.2 Hz, 1H), 8.03 (d, T=2.1 Hz, 1H), 7.54 (d, J=16.0 Hz, 1H), 6.51 (d, J=16.0 Hz, 1H), 2.91 (t, J=7.6 Hz, 2H), 2.53 (dd, J=8.5, 6.7 Hz, 2H). ¹³C NMR (126 MHz, DMSO) δ 171.03, 167.48, 152.74, 147.26, 140.61, 133.85, 124.75, 119.27, 118.40, 29.98, 23.28.

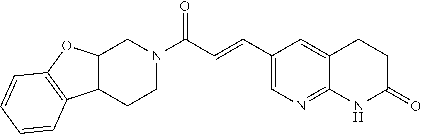 [SMILES: O=C1CCc2cc(/C=C/C(=O)N3CCC4c5ccccc5OC4C3)cnc2N1]

(E)-6-(3-0xo-3-(3,4,4a,9a-tetrahydrobenzofuro[2,3-c]pyridin-2(1H)-yl)prop-1-en-1-yl)-3,4-dihydro-1,8-naphthyridin-2(1H)-one. To a solution of 1,2,3,4-tetraydrobenzofuro[2,3-c] pyridine hydrochloride (1 eq, 0.36 mmol), (E)-3-(7-oxo-5,6,7,8-tetrahydro-1,8-naphthyridin-3-yl)acrylic acid hydrochloride (1.2 eq, 0.432 mmol), HOBt (1.2 eq, 0.432 mmol), in DMF (2.5 mL) was added EDC (1.2 eq, 0.432 mmol). The reaction mixture was heated to 85° C. for 6 h. The solvent was reduced to a few mL. The crude reaction mixture was diluted with CH₂Cl₂ and washed with saturated aqueous sodium bicarbonate. The organic extracts were dried over sodium sulfate and concentrated under reduced pressure. Purification by flash column chromatography (02:98 to 05:95, MeOH:CH₂Cl₂) yielded (E)-6-(3-oxo-3-(3,4,4a,9a-tetrahydrobenzofuro[2,3-c]pyridin-2(1H)-yl)prop-1-en-1-yl)-3,4-dihydro-1,8-naphthyridin-2(1H)-one (86 g, 0.23 mmol, 64%) as a white solid. ¹H NMR (500 MHz, Chloroform-d) δ 8.51-8.38 (m, 1H), 8.36 (s, 1H), 7.73-7.62 (m, 2H), 7.54-7.39 (m, 2H), 7.33-7.20 (m, 2H), 7.03-6.84 (m, 1H), 4.99-4.74 (m, 2H), 4.20-3.87 (m, 2H), 3.12-2.95 (m, 2H), 2.96-2.79 (m, 2H), 2.78-2.63 (m, 2H).

### Example 2

### Exemplary Analogs

The following naphthyridinone analogs can be prepared using the procedures described herein, including analogs having the (S)- or (R)-configuration.

R₁=R₂=H; R₁=H, R₂=CH₃; R₁=R₂=CH₃; R₁=H, R₂=C(O)CH₃; or R₁=H, R₂=Boc.

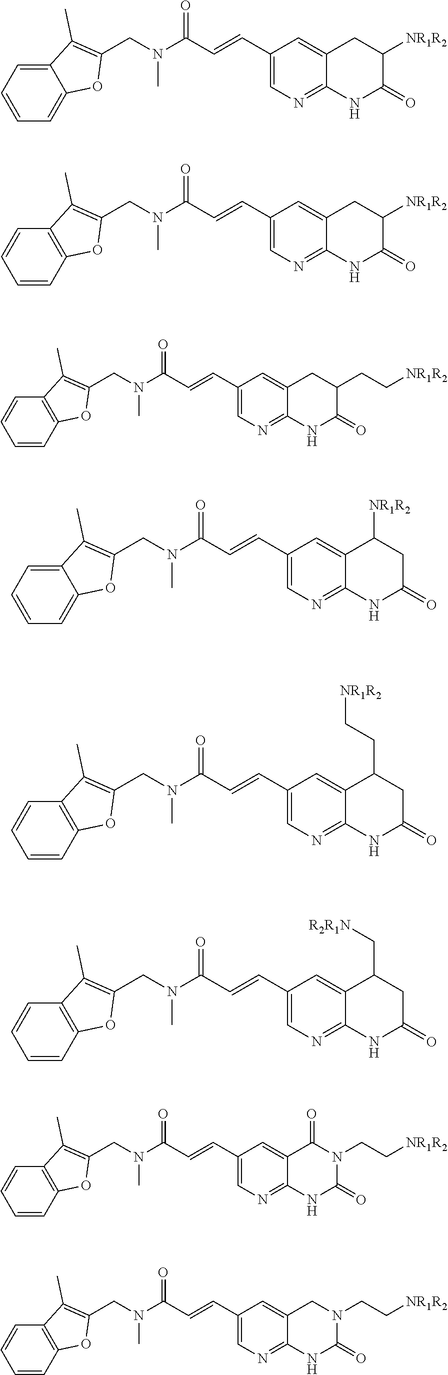

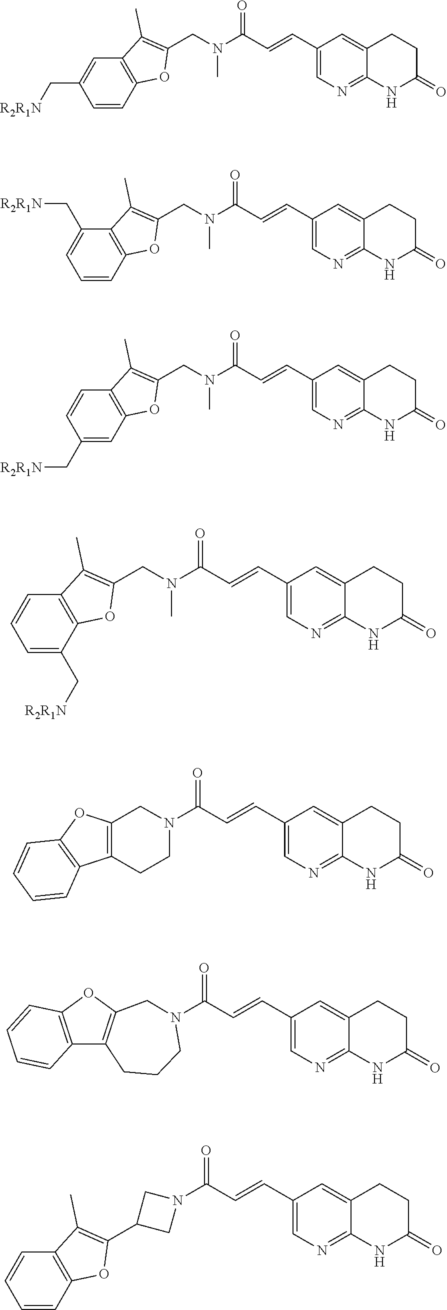

### Example 3

### Evaluation of Modified Debio-1452 Compounds Against Reference Strains

TABLE 8-1
Activity of cyclopentyl compounds (MIC ≤ 8 μg/mL).

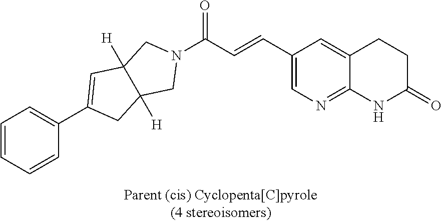 [SMILES: O=C1CCc2cc(/C=C/C(=O)N3CC4C=C(c5ccccc5)CC4C3)cnc2N1]

MIC S. aureus ATCC 29213 = 0.016 ug/mL
MIC E. coli ΔtolC mutant = ≤0.03 ug/mL
MIC E. coli ATCC 25922 = >32 ug/mL

| 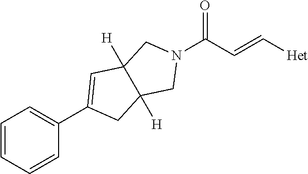 | 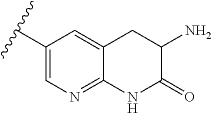 [SMILES: *c1cnc2c(c1)CC(N)C(=O)N2] | 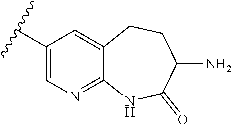 [SMILES: *c1cnc2c(c1)CCC(N)C(=O)N2] |
|---|---|---|
| Strain | (μg/mL) | (μg/mL) |
| S. aureus ATCC 29213 | 0.062 | 0.031 |
| E. coli ΔTolC | 0.062 | 0.062 |
| E. coli MG1655 | 16 | 8 |

TABLE 8-2
Activity of amine compounds.

| 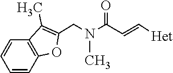 | 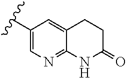 [SMILES: *c1cnc2c(c1)CCC(=O)N2] | 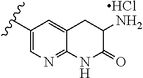 [SMILES: Cl ; *c1cnc2c(c1)CC(N)C(=O)N2] | 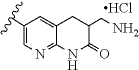 [SMILES: Cl ; *c1cnc2c(c1)CC(CN)C(=O)N2] | 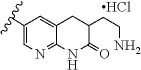 [SMILES: Cl ; *c1cnc2c(c1)CC(CCN)C(=O)N2] |
|---|---|---|---|---|
| Strain | (μg/mL) | (μg/mL) | (μg/mL) | (μg/mL) |
| S. aureus ATCC 29213 | 0.008-0.016 | 0.03125 | 0.031 | 0.016 |
| E. coli ΔTolC | 0.031 | 0.062 | 0.125 | 0.062 |
| E. coli MG1655 | >32 | 4 | 8 | 8 |

| 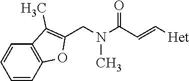 | 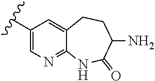 [SMILES: *c1cnc2c(c1)CCC(N)C(=O)N2] | 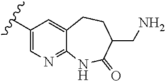 [SMILES: *c1cnc2c(c1)CCC(CN)C(=O)N2] | 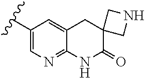 [SMILES: *c1cnc2c(c1)CC1(CNC1)C(=O)N2] |
|---|---|---|---|
| Strain | (μg/mL) | (μg/mL) | (μg/mL) |
| S. aureus ATCC 29213 | 0.016 | 0.031 | 0.016 |
| E. coli ΔTolC | 0.031 | 0.125 | 0.031 |
| E. coli MG1655 | 4 | 8 | 4 |
| 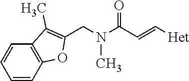 | 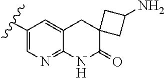 [SMILES: *c1cnc2c(c1)CC1(CC(N)C1)C(=O)N2] | 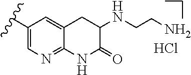 [SMILES: *c1cnc2c(c1)CC(NCCN)C(=O)N2 ; Cl] | 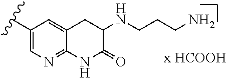 [SMILES: *c1cnc2c(c1)CC(NCCCN)C(=O)N2 ; "x HCOOH"] |
| Strain | (μg/mL) | (μg/mL) | (μg/mL) |
| S. aureus ATCC 29213 | 0.016 | 0.5 (80% @ 0.25) | 0.250 |
| E. coli ΔTolC | 0.062 | 1 (50% @ 0.5) | 0.5 |
| E. coli MG1655 | 4 | >32 (35% @ 32) | >32 |

TABLE 8-3
Antibacterial Susceptibility fluoro-substituted compounds.

| 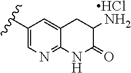 [SMILES: Cl ; *c1cnc2c(c1)CC(N)C(=O)N2] | 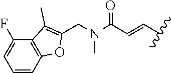 [SMILES: */C=C/C(=O)N(C)Cc1oc2cccc(F)c2c1C] | 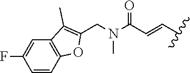 [SMILES: */C=C/C(=O)N(C)Cc1oc2ccc(F)cc2c1C] | 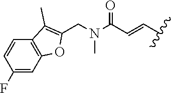 [SMILES: */C=C/C(=O)N(C)Cc1oc2cc(F)ccc2c1C] | 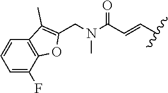 [SMILES: */C=C/C(=O)N(C)Cc1oc2c(F)cccc2c1C] |
|---|---|---|---|---|
| Strain | (μg/mL) | (μg/mL)** | (μg/mL)** | (μg/mL) |
| S. aureus ATCC | 0.031 | 0.125 (60% @ 0.062) | 0.062 | 0.016 |
| 29213 | | | | |
| E. coli ΔTolC | 0.125 | 1 (60% @ 0.5) | 0.5 | 0.062 |
| E. coli MG1655 | 8 | >32 (40% @ 32) | >32 (65% @ 32) | 8 (50-80% @ 4) |

### Example 4

### Evaluation of Modified Debio-1452 Compounds Against Additional Gram-Negative Clinical Isolates

TABLE 9
Activity Trends for Modified Debio1452 Compounds: MIC (μg/mL).

| 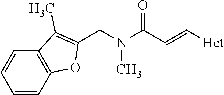 | 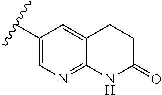 [SMILES: *c1cnc2c(c1)CCC(=O)N2] | 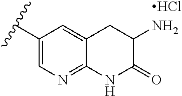 [SMILES: Cl ; *c1cnc2c(c1)CC(N)C(=O)N2] | 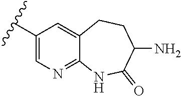 [SMILES: *c1cnc2c(c1)CCC(N)C(=O)N2] |
|---|---|---|---|
| Strain | Debio-1452 | Debio-1452-NH₃ | 2 stereoisomers |
| EC AR 0048 | >32 | 32 | 4 |
| EC AR 0085 | >32 | 16 | 4 |
| KP AR 0066 | >32 | 32 | 8 |
| KP AR 0113 | >32 | 32 | 8 |
| KP AR 0560 | >32 | 32 | 8 |
| KP BAA2472 | >32 | 16 | 8 |
| AB AR 0033 | >32 | 32 | 4 |
| AB AR 0273 | >32 | 32 | 4 |
| AB AR 0299 | >32 | 32 | 4 |
| AB AR 0313 | >32 | 32 | 4 |
| 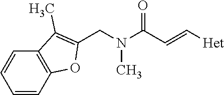 | | | |
| Strain | 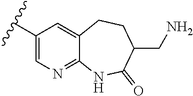 [SMILES: *c1cnc2c(c1)CCC(CN)C(=O)N2] | 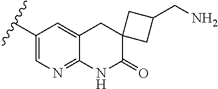 [SMILES: *c1cnc2c(c1)CC1(CC(CN)C1)C(=O)N2] | 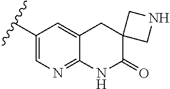 [SMILES: *c1cnc2c(c1)CC1(CNC1)C(=O)N2] |
| EC AR 0048 | 16 | 32 | 8 |
| EC AR 0085 | 16 | 16 | 4 |
| KP AR 0066 | 32 | 32 | 8 |
| KP AR 0113 | 32 | 32 | 16 |
| KP AR 0560 | 16 | 32 | 16 |
| KP BAA2472 | 32 | 32 | 8 |
| AB AR 0033 | 16 | 32 | 8 |
| AB AR 0273 | 32 | >32 | 16 |
| AB AR 0299 | 32 | >32 | 16 |
| AB AR 0313 | 32 | >32 | 16 |

### Example 5

### Evaluation Resolved Enantiomers

TABLE 10-1
Antibacterial Susceptibility of azepine isomers.

| 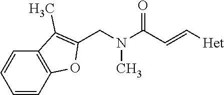 | 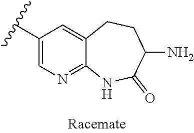 [SMILES: *c1cnc2c(c1)CCC(N)C(=O)N2] | 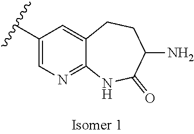 [SMILES: *c1cnc2c(c1)CCC(N)C(=O)N2] | 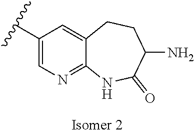 [SMILES: *c1cnc2c(c1)CCC(N)C(=O)N2] |
|---|---|---|---|
| Strain | (μg/mL) | (μg/mL) | (μg/mL) |
| S. aureus ATCC 29213 | 0.016 | 0.125-0.250 | 0.004-0.008 |
| E. coli ΔTolC | 0.031 | >1 (50% @ 1) | 0.031 (40-95% @ 0.016) |
| E. coli MG1655 | 4 | >32 (20% @ 32) | 2 |

TABLE 10-2
Activity Trends for Debio1452 compounds: MIC (μg/mL).

| | | | 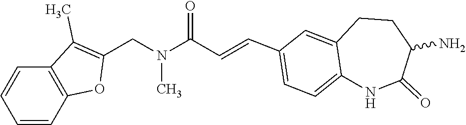 [SMILES: Cc1c(CN(C)C(=O)/C=C/c2ccc3c(c2)CCC(N)C(=O)N3)oc2ccccc12] | | |
|---|---|---|---|---|---|
| Strain | Debio-1452 | Debio-1452-NH₃ | 2 stereoisomers | Isomer 1 | Isomer 2 |
| EC AR 0048 | >32 | 32 | 4 | >32 | 4 |
| EC AR 0085 | >32 | 16 | 4 | >32 | 2 |
| KP AR 0066 | >32 | 32 | 8 | >32 | 4 |
| KP AR 0113 | >32 | 32 | 8 | >32 | 8 |
| KP AR 0560 | >32 | 32 | 8 | >32 | 4 |
| KP BAA2472 | >32 | 16 | 8 | >32 | 4 |
| AB AR 0033 | >32 | 32 | 4 | >32 | 2 |
| AB AR 0273 | >32 | 32 | 4 | >32 | 4 |
| AB AR 0299 | >32 | 32 | 4 | >32 | 2 |
| AB AR 0313 | >32 | 32 | 4 | >32 | 4 |

TABLE 10-3
Toxicity Screen Against Mammalian Cell Lines: Debio Antibiotics (72 h).

| | 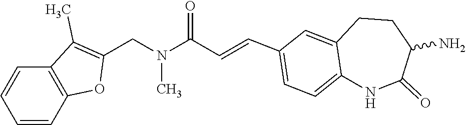 [SMILES: Cc1c(CN(C)C(=O)/C=C/c2ccc3c(c2)CCC(N)C(=O)N3)oc2ccccc12] | | Debio-1452-NH₃ | |
|---|---|---|---|---|
| Concentration (μM) | 30 | 100 | 30 | 100** |
| IMR90 % Cell Death | 8 ± 3 | 67 ± 2 | 18 ± 4 | 69 ± 5 |
| Hep G2 % Cell Death | 12 ± 5 | 58 ± 10 | 29 ± 8 | 77 ± 3 |

### Example 6

### Additional Compounds and Corresponding in-vitro Data

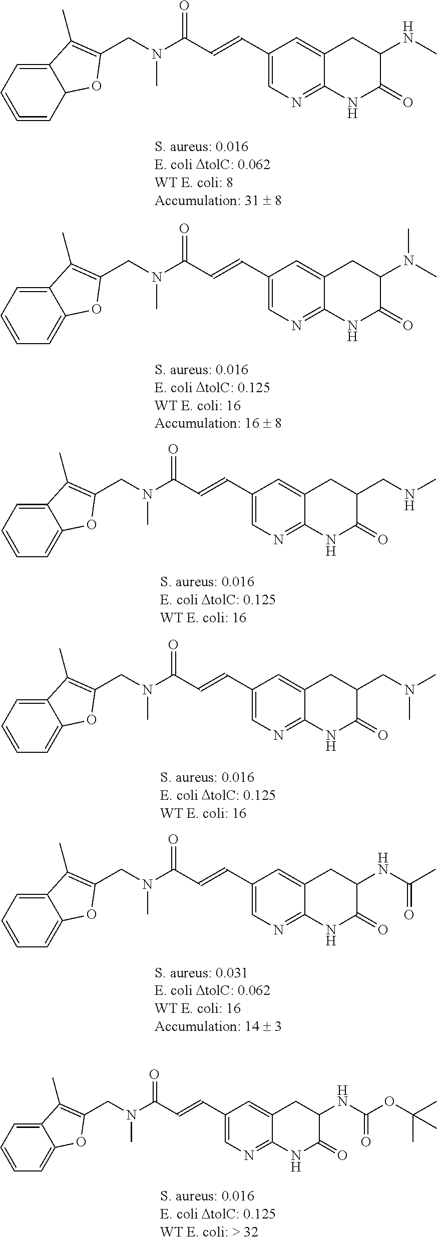 [SMILES: CNC1Cc2cc(/C=C/C(=O)N(C)CC3=C(C)C4=CC=C=CC4O3)cnc2NC1=O ; Cc1c(CN(C)C(=O)/C=C/c2cnc3c(c2)CC(N(C)C)C(=O)N3)oc2ccccc12 ; CNCC1Cc2cc(/C=C/C(=O)N(C)Cc3oc4ccccc4c3C)cnc2NC1=O ; Cc1c(CN(C)C(=O)/C=C/c2cnc3c(c2)CC(CN(C)C)C(=O)N3)oc2ccccc12 ; CC(=O)NC1Cc2cc(/C=C/C(=O)N(C)Cc3oc4ccccc4c3C)cnc2NC1=O ; Cc1c(CN(C)C(=O)/C=C/c2cnc3c(c2)CC(NC(=O)OC(C)(C)C)C(=O)N3)oc2ccccc12]

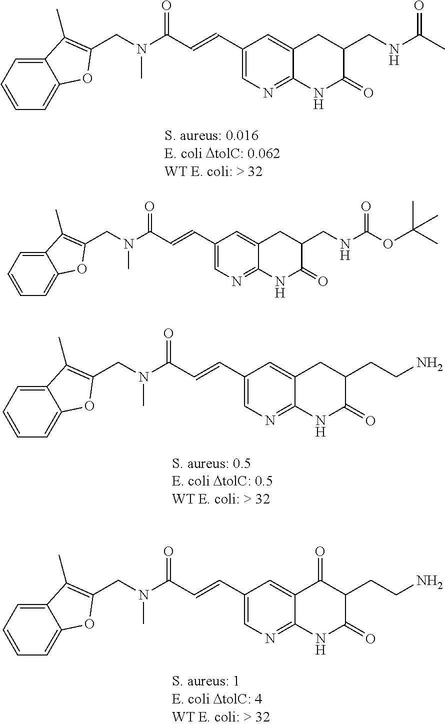 [SMILES: CC(=O)NCC1Cc2cc(/C=C/C(=O)N(C)Cc3oc4ccccc4c3C)cnc2NC1=O ; Cc1c(CN(C)C(=O)/C=C/c2cnc3c(c2)CC(CNC(=O)OC(C)(C)C)C(=O)N3)oc2ccccc12 ; Cc1c(CN(C)C(=O)/C=C/c2cnc3c(c2)CC(CCN)C(=O)N3)oc2ccccc12 ; Cc1c(CN(C)C(=O)/C=C/c2cnc3c(c2)C(=O)C(CCN)C(=O)N3)oc2ccccc12]

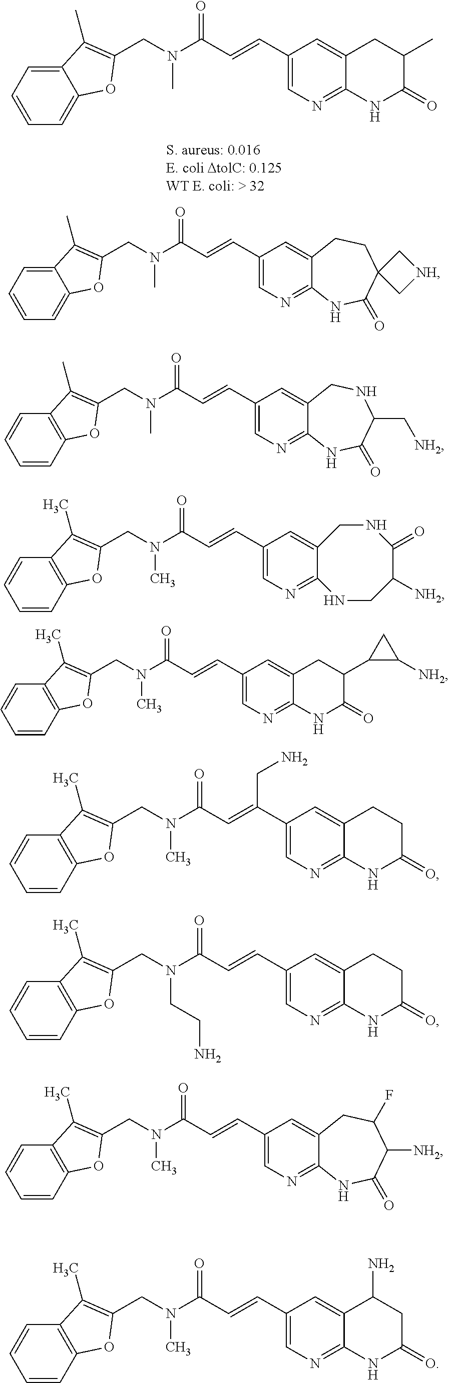

### Azepine Variants

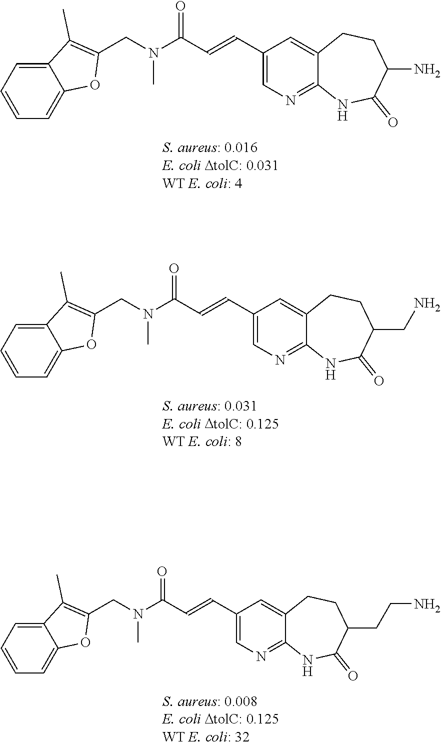 [SMILES: Cc1c(CN(C)C(=O)/C=C/c2cnc3c(c2)CCC(N)C(=O)N3)oc2ccccc12 ; Cc1c(CN(C)C(=O)/C=C/c2cnc3c(c2)CCC(CN)C(=O)N3)oc2ccccc12 ; Cc1c(CN(C)C(=O)/C=C/c2cnc3c(c2)CCC(CCN)C(=O)N3)oc2ccccc12]

### Spirocyclic Napthyridione Variants

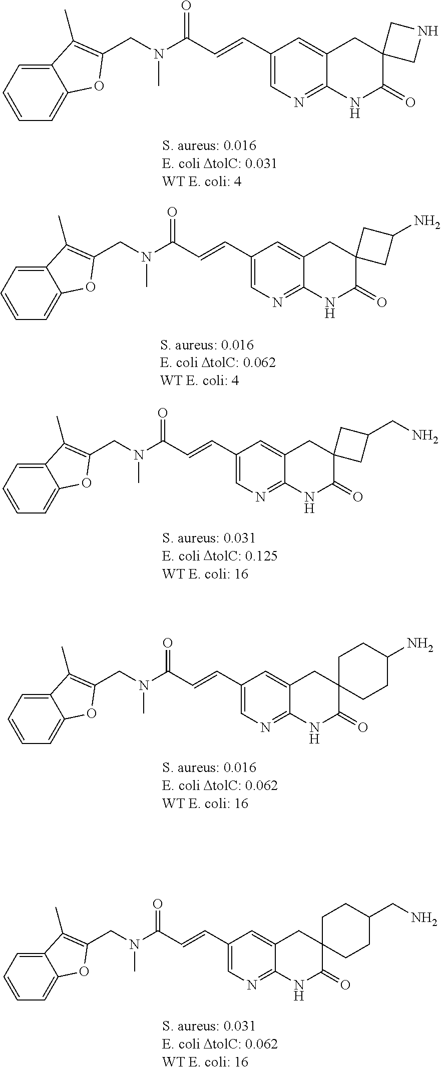 [SMILES: Cc1c(CN(C)C(=O)/C=C/c2cnc3c(c2)CC2(CNC2)C(=O)N3)oc2ccccc12 ; Cc1c(CN(C)C(=O)/C=C/c2cnc3c(c2)CC2(CC(N)C2)C(=O)N3)oc2ccccc12 ; Cc1c(CN(C)C(=O)/C=C/c2cnc3c(c2)CC2(CC(CN)C2)C(=O)N3)oc2ccccc12 ; Cc1c(CN(C)C(=O)/C=C/c2cnc3c(c2)CC2(CCC(N)CC2)C(=O)N3)oc2ccccc12 ; Cc1c(CN(C)C(=O)/C=C/c2cnc3c(c2)CC2(CCC(CN)CC2)C(=O)N3)oc2ccccc12]

### Napthyridione Diamine Variants

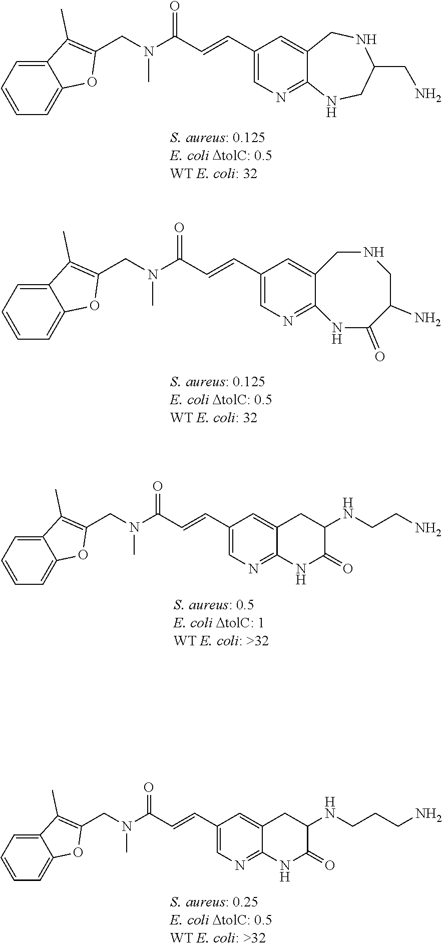 [SMILES: Cc1c(CN(C)C(=O)/C=C/c2cnc3c(c2)CNC(CN)CN3)oc2ccccc12 ; Cc1c(CN(C)C(=O)/C=C/c2cnc3c(c2)CNCC(N)C(=O)N3)oc2ccccc12 ; Cc1c(CN(C)C(=O)/C=C/c2cnc3c(c2)CC(NCCN)C(=O)N3)oc2ccccc12 ; Cc1c(CN(C)C(=O)/C=C/c2cnc3c(c2)CC(NCCCN)C(=O)N3)oc2ccccc12]

### Fused Diamine Variants

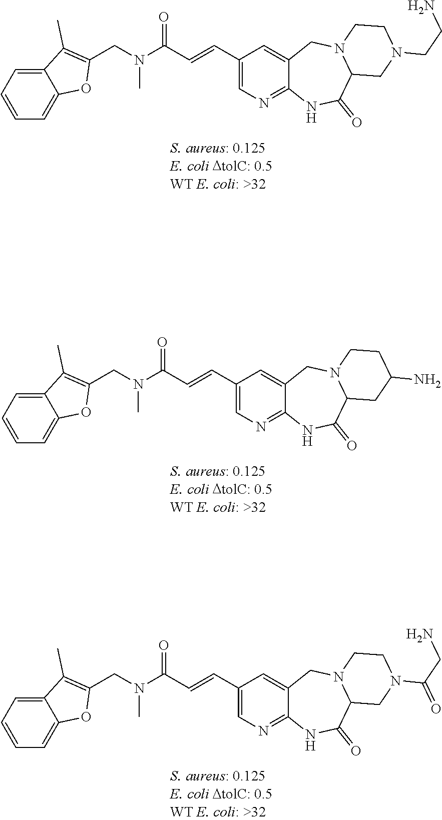 [SMILES: Cc1c(CN(C)C(=O)/C=C/c2cnc3c(c2)CN2CCN(CCN)CC2C(=O)N3)oc2ccccc12 ; Cc1c(CN(C)C(=O)/C=C/c2cnc3c(c2)CN2CCC(N)CC2C(=O)N3)oc2ccccc12 ; Cc1c(CN(C)C(=O)/C=C/c2cnc3c(c2)CN2CCN(C(=O)CN)CC2C(=O)N3)oc2ccccc12]

### Desmethyl Variants

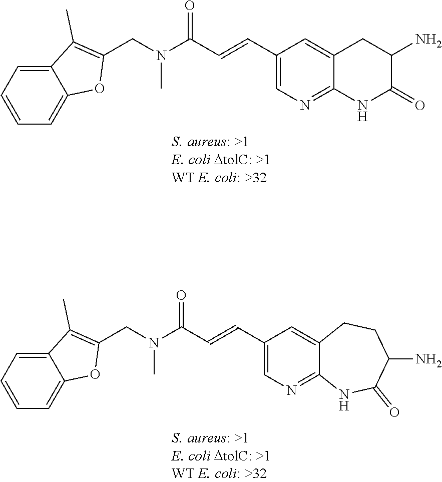 [SMILES: Cc1c(CN(C)C(=O)/C=C/c2cnc3c(c2)CC(N)C(=O)N3)oc2ccccc12 ; Cc1c(CN(C)C(=O)/C=C/c2cnc3c(c2)CCC(N)C(=O)N3)oc2ccccc12]

### Aminomethyl Benzofuran Variants

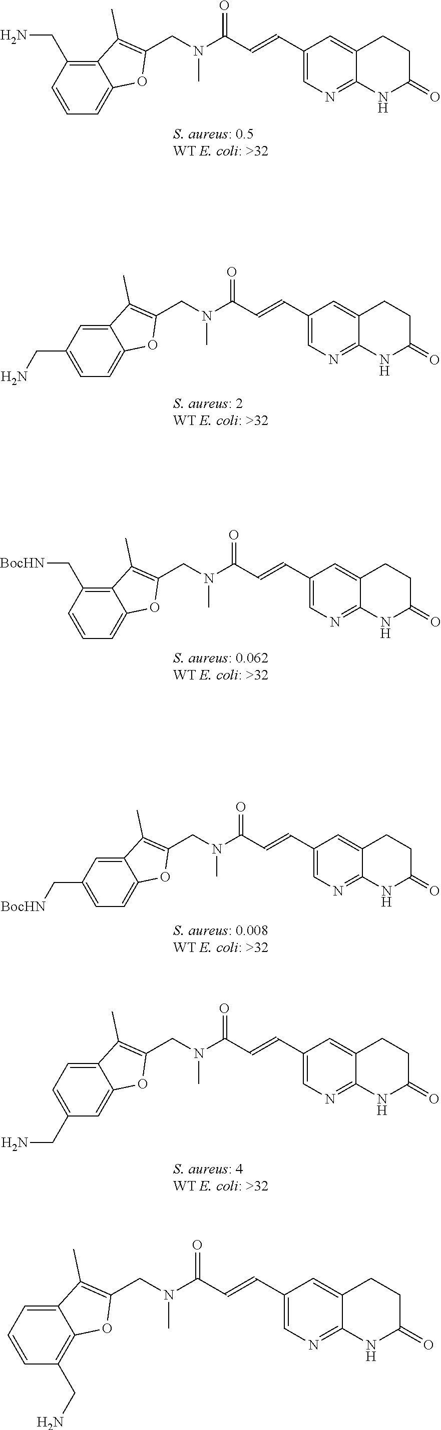 [SMILES: Cc1c(CN(C)C(=O)/C=C/c2cnc3c(c2)CCC(=O)N3)oc2cccc(CN)c12 ; Cc1c(CN(C)C(=O)/C=C/c2cnc3c(c2)CCC(=O)N3)oc2ccc(CN)cc12 ; Cc1c(CN(C)C(=O)/C=C/c2cnc3c(c2)CCC(=O)N3)oc2cccc(CNC(=O)OC(C)(C)C)c12 ; Cc1c(CN(C)C(=O)/C=C/c2cnc3c(c2)CCC(=O)N3)oc2ccc(CNC(=O)OC(C)(C)C)cc12 ; Cc1c(CN(C)C(=O)/C=C/c2cnc3c(c2)CCC(=O)N3)oc2cc(CN)ccc12 ; Cc1c(CN(C)C(=O)/C=C/c2cnc3c(c2)CCC(=O)N3)oc2c(CN)cccc12]

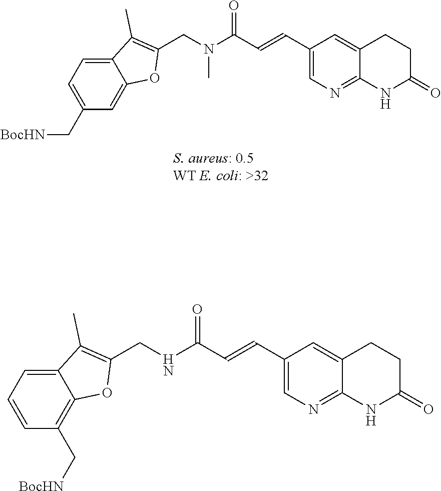 [SMILES: Cc1c(CN(C)C(=O)/C=C/c2cnc3c(c2)CCC(=O)N3)oc2cc(CNC(=O)OC(C)(C)C)ccc12 ; Cc1c(CNC(=O)/C=C/c2cnc3c(c2)CCC(=O)N3)oc2c(CNC(=O)OC(C)(C)C)cccc12]

### Aminopropynyl Benzofuran Variants

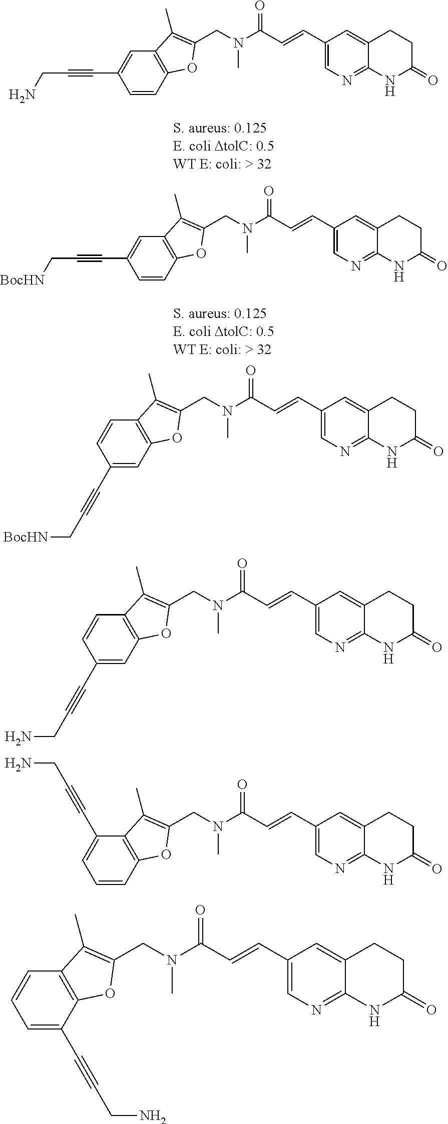

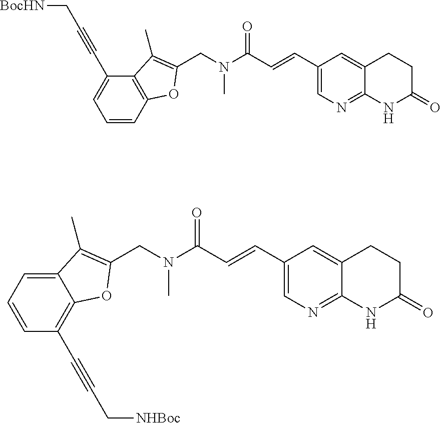 [SMILES: Cc1c(CN(C)C(=O)/C=C/c2cnc3c(c2)CCC(=O)N3)oc2cccc(C#CCNC(=O)OC(C)(C)C)c12 ; Cc1c(CN(C)C(=O)/C=C/c2cnc3c(c2)CCC(=O)N3)oc2c(C#CCNC(=O)OC(C)(C)C)cccc12]

### Cyclopenta[C]Pyrrolidine Variants

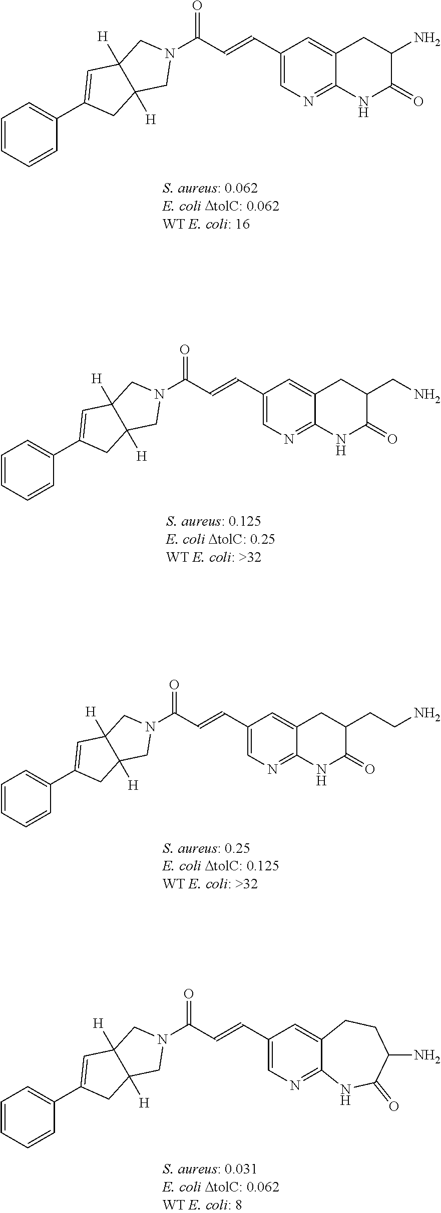 [SMILES: NC1Cc2cc(/C=C/C(=O)N3CC4C=C(c5ccccc5)CC4C3)cnc2NC1=O ; NCC1Cc2cc(/C=C/C(=O)N3CC4C=C(c5ccccc5)CC4C3)cnc2NC1=O ; NCCC1Cc2cc(/C=C/C(=O)N3CC4C=C(c5ccccc5)CC4C3)cnc2NC1=O ; NC1CCc2cc(/C=C/C(=O)N3CC4C=C(c5ccccc5)CC4C3)cnc2NC1=O]

### Azetidine and Phenylpiperidine Variants

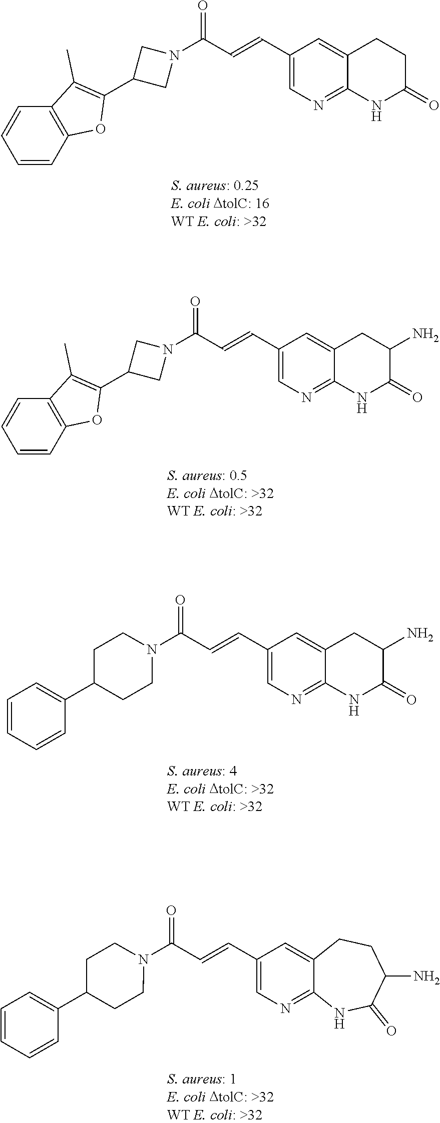 [SMILES: Cc1c(C2CN(C(=O)/C=C/c3cnc4c(c3)CCC(=O)N4)C2)oc2ccccc12 ; Cc1c(C2CN(C(=O)/C=C/c3cnc4c(c3)CC(N)C(=O)N4)C2)oc2ccccc12 ; NC1Cc2cc(/C=C/C(=O)N3CCC(c4ccccc4)CC3)cnc2NC1=O ; NC1CCc2cc(/C=C/C(=O)N3CCC(c4ccccc4)CC3)cnc2NC1=O]

### Fused Azepane and Phenylazepane Variants

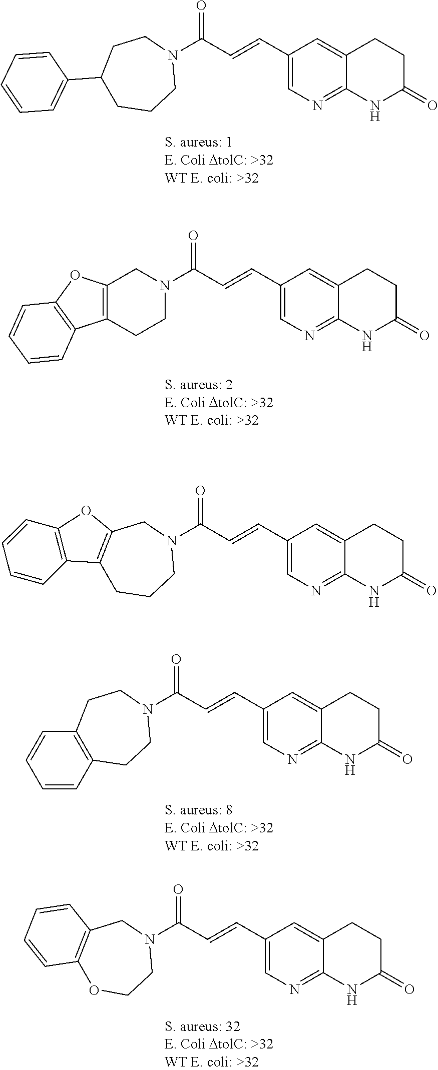 [SMILES: O=C1CCc2cc(/C=C/C(=O)N3CCCC(c4ccccc4)CC3)cnc2N1 ; O=C1CCc2cc(/C=C/C(=O)N3CCc4c(oc5ccccc45)C3)cnc2N1 ; O=C1CCc2cc(/C=C/C(=O)N3CCCc4c(oc5ccccc45)C3)cnc2N1 ; O=C1CCc2cc(/C=C/C(=O)N3CCc4ccccc4CC3)cnc2N1 ; O=C1CCc2cc(/C=C/C(=O)N3CCOc4ccccc4C3)cnc2N1]

### Fluorinated Variants

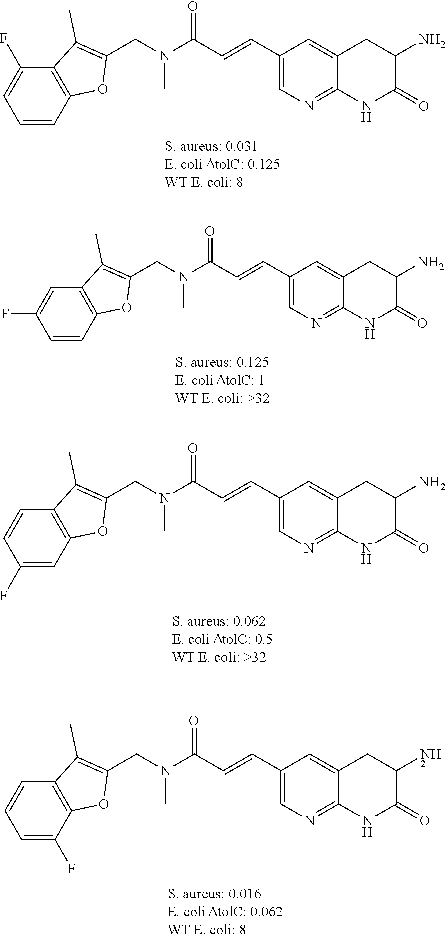 [SMILES: Cc1c(CN(C)C(=O)/C=C/c2cnc3c(c2)CC(N)C(=O)N3)oc2cccc(F)c12 ; Cc1c(CN(C)C(=O)/C=C/c2cnc3c(c2)CC(N)C(=O)N3)oc2ccc(F)cc12 ; Cc1c(CN(C)C(=O)/C=C/c2cnc3c(c2)CC(N)C(=O)N3)oc2cc(F)ccc12 ; Cc1c(CN(C)C(=O)/C=C/c2cnc3c(c2)CC(N)C(=O)N3)oc2c(F)cccc12]

### Example 7

### Pharmaceutical Dosage Forms

The following formulations illustrate representative pharmaceutical dosage forms that may be used for the therapeutic or prophylactic administration of a compound of a formula described herein, a compound specifically disclosed herein, or a pharmaceutically acceptable salt or solvate thereof (hereinafter referred to as ‘Compound X’):

| (i) Tablet 1 | mg/tablet |
|---|---|
| ‘Compound X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| ‘Compound X’ | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| ‘Compound X’ | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/mL) | mg/mL |
|---|---|
| ‘Compound X’ (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/mL) | mg/mL |
|---|---|
| ‘Compound X’ (free acid form) | 10.0 |
| Monobasic sodium phosphate | 0.3 |
| Dibasic sodium phosphate | 1.1 |
| Polyethylene glycol 400 | 200.0 |
| 0.1N Sodium hydroxide solution | q.s. |
| (pH adjustment to 7.0-7.5) | |
| Water for injection | q.s. ad 1 mL |

| (vi) Aerosol | mg/can |
|---|---|
| ‘Compound X’ | 20 |
| Oleic acid | 10 |
| Trichloromonofluoromethane | 5,000 |
| Dichlorodifluoromethane | 10,000 |
| Dichlorotetrafluoroethane | 5,000 |

| (vii) Topical Gel 1 | wt. % |
|---|---|
| ‘Compound X’ | 5% |
| Carbomer 934 | 1.25% |
| Triethanolamine | q.s. |
| (pH adjustment to 5-7) | |
| Methyl paraben | 0.2% |
| Purified water | q.s. to 100 g |

| (viii) Topical Gel 2 | wt. % |
|---|---|
| ‘Compound X’ | 5% |
| Methylcellulose | 2% |
| Methyl paraben | 0.2% |
| Propyl paraben | 0.02% |
| Purified water | q.s. to 100 g |

| (ix) Topical Ointment | wt. % |
|---|---|
| ‘Compound X’ | 5% |
| Propylene glycol | 1% |
| Anhydrous ointment base | 40% |
| Polysorbate 80 | 2% |
| Methyl paraben | 0.2% |
| Purified water | q.s. to 100 g |

| (x) Topical Cream 1 | wt. % |
|---|---|
| ‘Compound X’ | 5% |
| White bees wax | 10% |
| Liquid paraffin | 30% |
| Benzyl alcohol | 5% |
| Purified water | q.s. to 100 g |

| (xi) Topical Cream 2 | wt. % |
|---|---|
| ‘Compound X’ | 5% |
| Stearic acid | 10% |
| Glyceryl monostearate | 3% |
| Polyoxyethylene stearyl ether | 3% |
| Sorbitol | 5% |
| Isopropyl palmitate | 2% |
| Methyl Paraben | 0.2% |
| Purified water | q.s. to 100 g |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Compound X’. Aerosol formulation (vi) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed is:

1. A compound of Formula I or II: 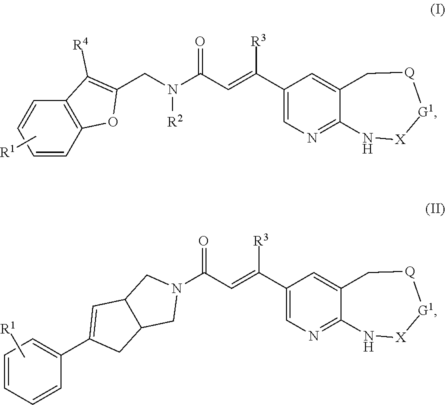
or a salt thereof;
wherein
G¹ is —CH(C₀-C₆)alkyl-NH₂, CH₂, —CH(C₃-C₆)cycloalkyl-NH₂, —CHN(H)(C₂-C₆)alkyl-NH₂, or G²;
G² is 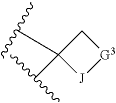
wherein G³ is NH, —CH(C₀-C₆)alkyl-NH₂, or —CH₂CH(C₀-C₆)alkyl-NH₂;
J is —(CH₂)ₘ—; and
m is 1-3;
Q is CHR^{a}, NH, —NHCH₂—, —CH₂CH₂—, —C(═O)N—, or absent, wherein R^{a} is H or halo;
X is C═O or CH₂;
R¹ is H or halo;
R² is —(C₁-C₆)alkyl, H, or —(C₁-C₆)alkyl-NH₂;
R³ is H or —(C₁-C₆)alkyl-NH2; and
R⁴ is methyl, ethyl, propyl, butyl, pentyl, hexyl, or —(C₃-C₆)cycloalkyl;
wherein for Formula I, R² and R⁴ taken together optionally form a heterocycle; and
wherein at least one of G¹, R², or R³ comprises an amine moiety and each alkyl moiety is optionally substituted.

2. The compound of claim 1 wherein G¹ is G².

3. The compound of claim 1 wherein the compound is represented by Formula I, Q is absent, and R² or R³ is —(C₁-C₆)alkyl-NH₂.

4. The compound of claim 1 wherein R¹ is fluoro.

5. The compound of claim 1 wherein the compound is represented by Formula IA: 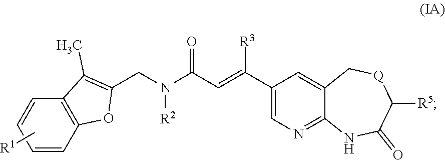
wherein
R⁵ is —(C₀-C₆)alkyl-NH₂ or —NH(C₂-C₆)alkyl-NH₂.

6. The compound of claim 5 wherein Q is CH₂ or CHF.

7. The compound of claim 5 wherein Q is CH₂, R¹ is H or fluoro, R² is methyl, R³ is H, and R⁵ is NH₂ or CH₂NH₂.

8. The compound of claim 5 wherein the compound is:

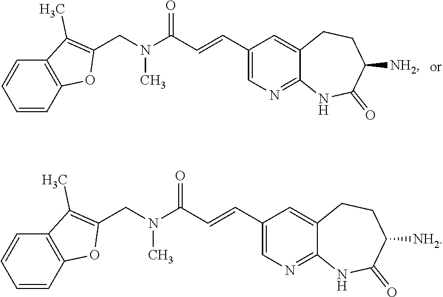 [SMILES: Cc1c(CN(C)C(=O)/C=C/c2cnc3c(c2)CC[C@@H](N)C(=O)N3)oc2ccccc12 ; Cc1c(CN(C)C(=O)/C=C/c2cnc3c(c2)CC[C@H](N)C(=O)N3)oc2ccccc12]

9. The compound of claim 1 wherein Q is absent, R¹ is fluoro, R² is methyl, R³ is H, and R⁵ is —NHCH₂CH₂NH₂ or —NHCH₂CH₂CH₂NH₂.

10. The compound of claim 1 wherein the compound is represented by Formula IB: 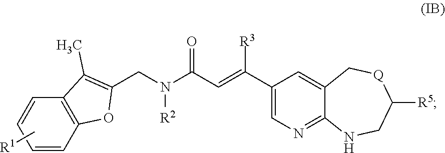
wherein
Q is NH, or —C(═O)N— wherein the carbonyl moiety of the amide group is at the position alpha to the tertiary carbon that is substituted with R⁵; and
R⁵ is —(C₀-C₆)alkyl-NH₂ or —NH(C₂-C₆)alkyl-NH₂.

11. The compound of claim 1 wherein the compound is represented by Formula IC:

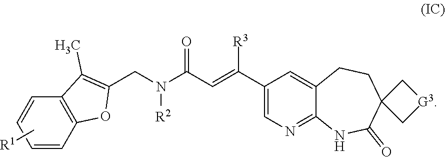

12. The compound of claim 11 wherein G³ is NH.

13. The compound of claim 1 wherein the compound is represented by Formula ID:

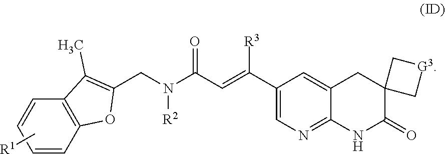

14. The compound of claim 13 wherein G³ is NH.

15. The compound of claim 13 wherein G³ is —CH(C₀-C₆)alkyl-NH₂.

16. The compound of claim 13 wherein G³ is NH, —CHNH₂, or —CHCH₂NH₂, R¹ is fluoro, R² is methyl, and R³ is H.

17. The compound of claim 1 wherein the compound is:

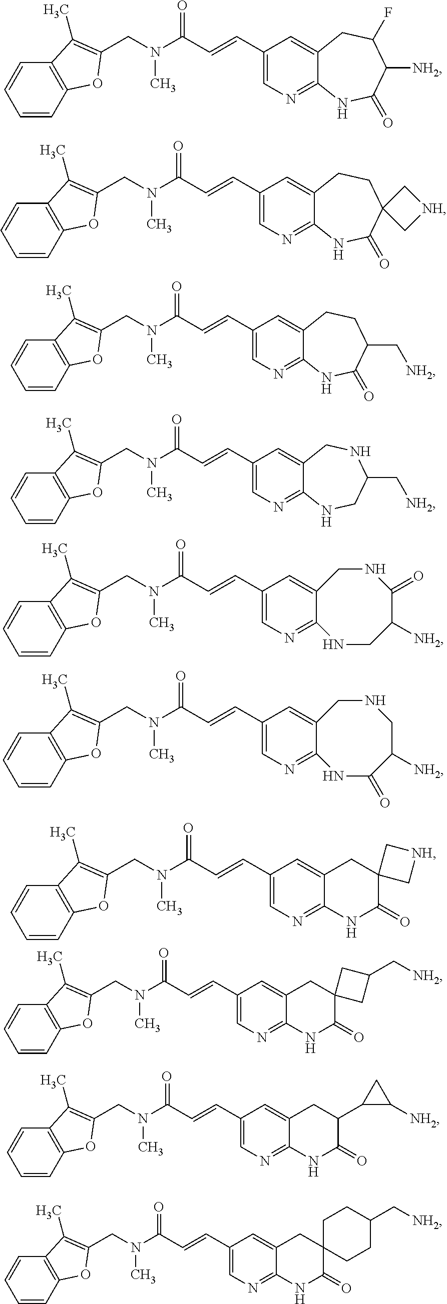

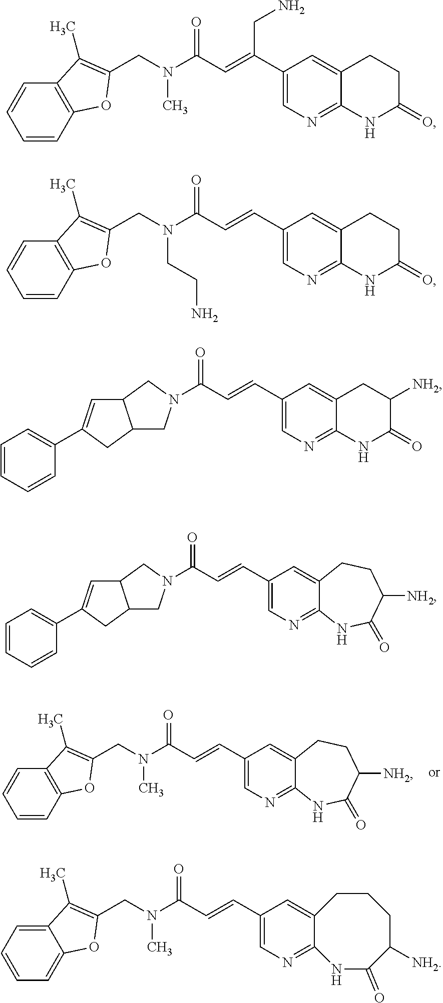

18. A compound of Formula III: 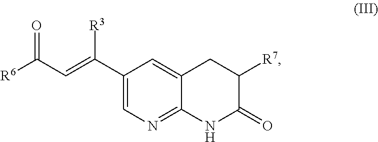
or a salt thereof;
wherein
R³ is H or —(C₁-C₆)alkyl;
R⁶ is 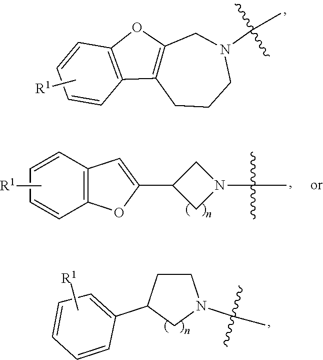
wherein R¹ is H or halo, and n is 1-4; and
R⁷ is H or —(C₀-C₆)alkyl-NH₂.

19. The compound of claim 18 wherein the compound is:

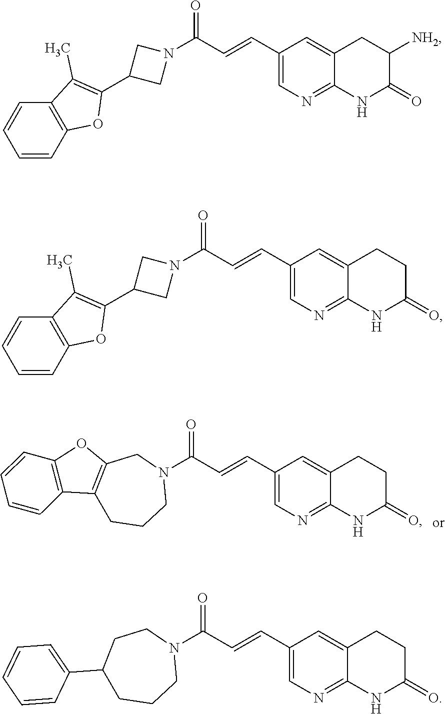 [SMILES: Cc1c(C2CN(C(=O)/C=C/c3cnc4c(c3)CC(N)C(=O)N4)C2)oc2ccccc12 ; Cc1c(C2CN(C(=O)/C=C/c3cnc4c(c3)CCC(=O)N4)C2)oc2ccccc12 ; O=C1CCc2cc(/C=C/C(=O)N3CCCc4c(oc5ccccc45)C3)cnc2N1 ; O=C1CCc2cc(/C=C/C(=O)N3CCCC(c4ccccc4)CC3)cnc2N1]

20. A compound of Formula IV: 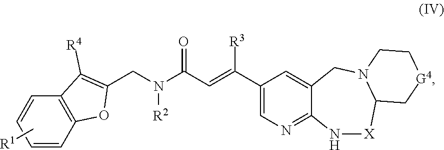
or a salt thereof;
wherein
G⁴ is O, NH, CH₂, —CH(C₀-C₆)alkyl-NH₂, —N(C═O)(C₁-C₆)alkyl-NH₂;
X is C═O or CH₂;
R¹ is H or halo;
R² is H, —(C₁-C₆)alkyl, or —(C₁-C₆)alkyl-NH₂;
R³ is H or —(C₁-C₆)alkyl-NH₂; and
R⁴ is methyl, ethyl, or propyl;
wherein at least one of G⁴, R², or R³ comprises an amine moiety.

21. The compound of claim 20 wherein the compound is:

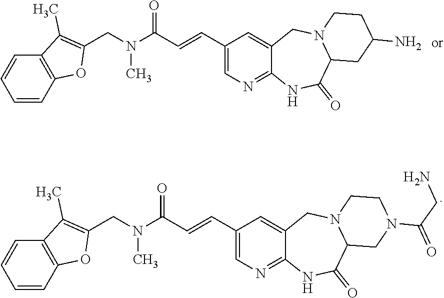 [SMILES: Cc1c(CN(C)C(=O)/C=C/c2cnc3c(c2)CN2CCC(N)CC2C(=O)N3)oc2ccccc12 ; Cc1c(CN(C)C(=O)/C=C/c2cnc3c(c2)CN2CCN(C(=O)CN)CC2C(=O)N3)oc2ccccc12]

22. (canceled)

23. A method of antimicrobial treatment comprising administering to a subject in need thereof a therapeutically effective amount of a compound of claim 1 or a pharmaceutically acceptable salt thereof, thereby killing or inhibiting the growth of at least a portion of a plurality of microorganisms in the subject.

24-27. (canceled)
